(19) United States
(12) Patent Application Publication
(10) Pub. No.: US 2014/0315844 A1
(43) Pub. Date: Oct. 23, 2014

(54) MULTI-GENE SIGNATURES FOR PREDICTING RESPONSE TO CHEMOTHERAPY OR RISK OF METASTASIS FOR BREAST CANCER

(71) Applicant: Celera Corporation, Alameda, CA (US)

(72) Inventors: Shirley KWOK, San Ramon, CA (US); Alice WANG, Lafayette, CA (US)

(73) Assignee: Celera Corporation, Alameda, CA (US)

(21) Appl. No.: 14/091,790

(22) Filed: Nov. 27, 2013

Related U.S. Application Data
(60) Provisional application No. 61/731,860, filed on Nov. 30, 2012.

(51) Int. Cl. C12Q 1/68

(52) U.S. Cl. CPC C12Q 1/6886
USPC 514/34; 514/648; 506/16; 506/9; 514/110; 514/249

(57) ABSTRACT

Exemplary embodiments of the invention provide methods and compositions relating to a multi-gene signature, and subsets thereof, for predicting whether an individual with breast cancer will respond to chemotherapy based on expression of the genes in the multi-gene signature, as well as for prognosing risk of breast cancer metastasis.

### CROSS-REFERENCE TO RELATED APPLICATIONS

This application is a non-provisional application which claims priority to U.S. provisional application Ser. No. 61/731,860, filed Nov. 30, 2012, the contents of which are hereby incorporated by reference in its entirety into this application.

### FIELD OF THE INVENTION

The present invention relates to breast cancer, particularly responsiveness to breast cancer chemotherapy and risk for metastasis of breast cancer. In exemplary embodiments, the invention provides methods and compositions relating to a multi-gene signature, and subsets thereof, for predicting whether an individual with breast cancer will respond to (benefit from) chemotherapy for treating breast cancer. In certain embodiments, methods and compositions are provided which relate to subsets of the multi-gene signature for predicting responsiveness to breast cancer chemotherapy and/or determining risk for breast cancer metastasis.

### BACKGROUND OF THE INVENTION

A 14-gene prognostic gene signature and a metastasis score (MS) derived therefrom, and their use for determining risk of distant metastasis in a breast cancer patient, have previously been described in U.S. Pat. No. 7,695,915 (issued Apr. 13, 2010 to Kit Lau et al.) and Tutt et al., “Risk estimation of distant metastasis in node-negative, estrogen receptor-positive breast cancer patients using an RT-PCR based prognostic expression signature”, BMC Cancer. 2008 Nov. 21; 8:339, both of which are incorporated herein by reference in their entirety. The MS, which was derived from and validated in untreated women, is prognostic of breast cancer distant metastasis in women with ER+N− tumors (Tutt et al., BMC Cancer. 2008 Nov. 21; 8:339). An independent assessment of the gene signature using public breast cancer microarray datasets showed that the genes that comprise the MS were highly significant in predicting distant metastasis-free survival in ER+/HER2− tumors (Esserman et al., Breast Cancer Res Treat (2011) 129(2):607-16).

Current breast cancer treatment guidelines in the United States and Europe recommend chemotherapy (the use of cytotoxic drugs) for the majority of patients with ER positive (ER+), node-positive (N+) breast cancer. However, the benefit of chemotherapy in ER+, node-negative (N−) breast cancer is less clear, particularly for postmenopausal women. Examples of chemotherapy regimens for treating breast cancer include, but are not limited to, CMF (cyclophosphamide, methotrexate, and fluorouracil (5-FU)) and CAF (cyclophosphamide, Adriamycin® (doxorubicin), and fluorouracil (5-FU)). Chemotherapy may be used in conjunction with tamoxifen treatment (for example, chemotherapy may precede, follow, or be concomitant with tamoxifen treatment of a breast cancer patient).

However, only a fraction of patients benefit from chemotherapy while the majority suffer unnecessarily from toxic side effects of the drugs. Chemotherapy, since it targets non-cancerous cells (particularly fast-dividing cells) in addition to cancerous cells, leads to common side effects such as myelosuppression (decreased production of blood cells, thereby leading to immunosuppression), mucositis (inflammation of the lining of the digestive tract), and alopecia (hair loss).

Anthracyclines (e.g., doxorubicin and epirubicin) are among the most effective class of chemotherapeutic agents for the treatment of breast cancer but they are notorious for causing serious cardiotoxicity, including congestive heart failure, particularly in older women. Indeed, reports indicate under-utilization of CAF chemotherapy for this reason. In a 10 year follow-up study, women aged 66 to 70 years who received anthracyclines experienced higher rates of heart failure than did women who received nonanthracyclines or no chemotherapy (Pinder et al., J Clin Oncol. 2007; 25:3808-3815).

Since individuals can respond differently to chemotherapy, it would be desirable to identify those individuals who would be most likely to benefit from chemotherapy so that they can be promptly treated with chemotherapy despite the potential side effects, while those individuals who would not benefit from chemotherapy can avoid suffering its side effects while receiving little or no benefit from the chemotherapy and could instead be given alternative treatments.

Furthermore, postmenopausal women, while representing the largest fraction of women diagnosed with primary breast cancer, are generally less responsive to chemotherapy and are more likely to experience toxic side effects than younger women. For example, in a 12 year follow-up of patients enrolled in NSABP20 (National Surgical Adjuvant Breast and Bowel Project), no advantage was observed in recurrence-free survival or overall survival when cyclophosphamide, methotrexate and 5-fluorouracil (CMF) chemotherapy was given with tamoxifen in women who were 60 years of age or older at the time of enrollment. In contrast, younger women (particularly those aged 49 years and younger) who were treated with both CMF and tamoxifen had significantly better outcomes relative to those who only received tamoxifen (Fisher et al., Lancet 2004 Sep. 4-10; 364(9437):858-68). In 2002, the International Breast Cancer Study Group (IBCSG) published the results of “Trial IX” which evaluated the role of adjuvant chemotherapy preceding treatment with tamoxifen for postmenopausal patients (median age of 61 years) with node negative disease (IBCSG, “Endocrine responsiveness and tailoring adjuvant therapy for postmenopausal lymph node-negative breast cancer: a randomized trial”, J Natl Cancer Inst. 2002; 94(14):1054-65 and, subsequently, Aebi et al., “Differential efficacy of three cycles of CMF followed by tamoxifen in patients with ER-positive and ER-negative tumors: long-term follow up on IBCSG Trial IX”, Ann Oncol. 2011, (9):1981-7). The Trial IX study showed that postmenopausal patients with ER+ tumors showed no benefit from the combined treatment of CMF and tamoxifen compared with tamoxifen alone, even though patients with ER− tumors did benefit significantly from adjuvant CMF chemotherapy preceding tamoxifen treatment.

Thus, because older postmenopausal women (who represent the largest fraction of women diagnosed with breast cancer) are less likely to respond to chemotherapy while also being more likely to experience toxic side effects, it is even more desirable to identify women in this patient population who would be more likely to respond to chemotherapy.

Thus, there is a need for diagnostic assays that can predict whether a breast cancer patient will respond to (benefit from) chemotherapy, particularly for postmenopausal women.

### SUMMARY OF EXEMPLARY EMBODIMENTS OF THE INVENTION

The present invention relates to responsiveness to breast cancer chemotherapy, as well as risk for metastasis of breast cancer. In exemplary embodiments, the present invention provides compositions and methods relating to a multi-gene signature (which may be interchangeably referred to herein as a “signature”, “molecular signature”, “expression signature”, “prognostic signature”, “predictive signature”, “combination”, or “panel”) for predicting whether an individual with breast cancer will respond to (benefit from) chemotherapy (e.g., CMF, CAF, AC, AC-Taxol®, or other chemotherapies) for treating breast cancer. In certain embodiments, the individual is a postmenopausal woman (as just one representative example, a postmenopausal woman may be, for example, at least 50, 51, 52, or 53 years of age or older). In certain exemplary embodiments, the multi-gene signature comprises, or consists of, the following 14 genes: CENPA, PKMYT1, MELK, MYBL2, BUB1, RACGAP1, TK1, UBE2S, DC13, RFC4, PRR11 (also known as F1111029), DIAPH3, ORC6L, and CCNB 1 (see Table 2 below). Certain embodiments also provide a “metastasis score” (“MS”) (which may also be referred to herein as a “score”), methods of calculating a metastasis score, and methods of applying/using a metastasis score (e.g., to predict response to chemotherapy and/or to determine risk for breast cancer metastasis). The 14-gene signature, and exemplary methods for deriving metastasis scores, are also described in U.S. Pat. No. 7,695,915 (issued Apr. 13, 2010 to Kit Lau et al.); Tutt et al., “Risk estimation of distant metastasis in node-negative, estrogen receptor-positive breast cancer patients using an RT-PCR based prognostic expression signature”, BMC Cancer. 2008 Nov. 21; 8:339; and U.S. Pat. No. 8,557,525 (Wang et al., “A Composite Metastasis Score with Weighted Coefficients”, filed Feb. 27, 2012, issued Oct. 15, 2013), each of which is incorporated herein by reference in its entirety. In further exemplary embodiments, the multi-gene signature comprises, or consists of, a subset of these 14 genes (such as any 5, 6, 7, 8, 9, 10, 11, 12, or 13 of these 14 genes). For example, in certain exemplary embodiments, the multi-gene signature comprises, or consists of, the following 12 genes: CENPA, PKMYT1, MELK, BUB1, RACGAP1, TK1, UBE2S, DC13, RFC4, PRR11, DIAPH3, and ORC6L (which may be referred to herein as the “12-gene signature”, and is shown in bold on page 2 of Table 40). In further exemplary embodiments, the multi-gene signature comprises these 12 (or other subset) or 14 genes plus additional genes (e.g., additional genes that are associated with risk for breast cancer metastasis and/or response to chemotherapy).

In certain exemplary embodiments, the chemotherapy comprises at least one of an anthracycline (e.g., doxorubicin and/or epirubicin), a taxane (e.g., paclitaxel and/or docetaxel), an inhibitor of nucleotide synthesis (e.g., methotrexate and/or 5-fluorouracil (5-FU)), and/or an alkylating agent (e.g., cyclophosphamide).

In certain exemplary embodiments, the chemotherapy comprises a chemotherapy such as CMF (cyclophosphamide, methotrexate, fluorouracil (5-FU)), CAF (also known as FAC) (cyclophosphamide, Adriamycin® (doxorubicin), fluorouracil (5-FU)), AC (also known as CA) [Adriamycin® (doxorubicin) and cyclophosphamide] or AC-Taxol® (AC followed by paclitaxel), as well as other chemotherapy regimens comprising any one or more of the individual chemotherapeutic agents which make up either of the aforementioned chemotherapy regimens. In certain embodiments, a breast cancer patient is treated with chemotherapy in addition to tamoxifen (e.g., chemotherapy treatment precedes tamoxifen treatment of an individual with breast cancer, or chemotherapy treatment follows tamoxifen treatment, or chemotherapy is given concomitantly with tamoxifen treatment).

In further exemplary embodiments, the chemotherapy regimen can comprise another regimen other than CMF, CAF, AC, or AC-Taxol®, such as, for example: TAC [Taxotere® (docetaxel), Adriamycin® (doxorubicin), and cyclophosphamide)], EC (epirubicin and cyclophosphamide), FEC (5-fluorouracil, epirubicin and cyclophosphamide), FECD (FEC followed by docetaxel), TC [Taxotere® (docetaxel) and cyclophosphamide], or MF (methotrexate and fluorouracil (5-FU)), as well as other chemotherapy regimens comprising any one or more of the individual chemotherapeutic agents which make up any of the aforementioned chemotherapy regimens. Any chemotherapy regimens can optionally include paclitaxel (e.g., Taxol® or Abraxane®) and/or docetaxel (e.g., Taxotere®), beyond those mentioned above. Furthermore, any chemotherapy regimens (including CMF and CAF) can also optionally further include one or more non-chemotherapy agents such as trastuzumab (Herceptin®) in combination with the chemotherapy.

Exemplary embodiments of the invention provide methods for predicting response to chemotherapy in an individual who has breast cancer, particularly wherein the individual is a postmenopausal woman. Certain exemplary embodiments of the invention provide methods for predicting risk of metastasis, particularly distant metastasis, in an individual who has breast cancer. In certain aspects of any of these embodiments relating to either chemotherapy response and/or metastasis risk, the breast tumor is estrogen receptor (ER)-positive and, in certain aspects, the breast tumor is HER2-negative. Furthermore, in certain aspects of any of these embodiments, the individual's breast cancer is lymph node-negative.

In certain exemplary embodiments of the invention, the response to breast cancer chemotherapy as predicted by gene expression analysis of the 14-gene signature (or a subset thereof such as the 12-gene signature of CENPA, PKMYT1, MELK, BUB1, RACGAP1, TK1, UBE2S, DC13, RFC4, PRR11, DIAPH3, and ORC6L, or the 12 or 14 genes plus additional genes), such as by determining a metastasis score (MS), is negatively (inversely) correlated with risk for breast cancer metastasis as predicted by the MS (or by gene expression analysis without deriving a MS), particularly in instances wherein the patient is a postmenopausal woman. Thus, in certain embodiments, an individual with breast cancer who is predicted to be at increased risk for tumor metastasis based on their MS (or by gene expression analysis without deriving an MS) is also predicted to be less likely to respond to (i.e., benefit from) chemotherapy (such as, but not limited to, CMF or CAF chemotherapy). Alternatively, an individual with breast cancer who is predicted to be at less risk for tumor metastasis based on their MS (or by gene expression analysis without deriving an MS) is also predicted to be more likely to respond to chemotherapy (such as, but not limited to, CMF or CAF chemotherapy). Thus, in certain embodiments, a low MS (or gene expression which is not increased) indicates an increased likelihood of responding to chemotherapy (and a decreased risk for metastasis), whereas a high MS (or increased gene expression) indicates a decreased likelihood of responding to chemotherapy (and an increased risk for metastasis) for a breast cancer patient (particularly a postmenopausal patient).

Similarly, in certain exemplary embodiments, the response to breast cancer chemotherapy as predicted by gene expression analysis of any one or more of the genes of the 14-gene signature is negatively (inversely) correlated with risk for breast cancer metastasis as predicted by expression of that gene, particularly in instances wherein the patient is a postmenopausal woman. For example, in certain embodiments, the response to breast cancer chemotherapy as predicted by expression of the MYBL2 gene is negatively (inversely) correlated with risk for breast cancer metastasis as predicted by expression of MYBL2, particularly in instances wherein the subject is a postmenopausal woman.

Prediction of chemotherapy response based on gene expression analysis of the 14-gene signature (or a subset thereof, or the 14 genes plus additional genes) can be independent of any assessment of risk. Thus, it is not necessary that prediction of chemotherapy response be coupled with a determination of metastasis risk. Determination of risk for breast cancer metastasis does not necessarily need to be done at all in order to determine response to chemotherapy such as chemotherapy. Rather, gene expression analysis of the 14 genes (or a subset thereof, or the 14 genes plus additional genes) can be carried out solely for the purpose of predicting response to chemotherapy. For example, in certain embodiments, a metastasis score (MS) can be derived from gene expression analysis of the 14 genes (or a subset thereof, or the 14 genes plus additional genes) of a breast cancer patient, and a low MS value (e.g., an MS value that is below a certain cutoff/threshold value) indicates that the breast cancer patient has an increased likelihood of responding to (benefitting from) chemotherapy, whereas a high MS value (e.g., an MS value that is above a certain cutoff/threshold value) indicates that the breast cancer patient has a decreased likelihood of responding to (benefitting from) chemotherapy. Embodiments such as these do not require any inquiry into metastasis risk (although determination of metastasis risk can also optionally be included as part of the analysis).

In exemplary embodiments, the multi-gene signature comprises, or consists of, a 14-gene signature for predicting response to breast cancer chemotherapy. The 14 genes in exemplary embodiments of the multi-gene signature are disclosed in Table 2. In certain embodiments, the multi-gene signature comprises, or consists of, a subset of the 14 genes provided in Table 2 (e.g., any 5, 6, 7, 8, 9, 10, 11, 12, or 13 of these genes), such as the subsets provided in Table 40. For example, particular embodiments comprise or consist of all 12 of the genes CENPA, PKMYT1, MELK, BUB1, RACGAP1, TK1, UBE2S, DC13, RFC4, PRR11, DIAPH3, and ORC6L. In certain embodiments, the multi-gene signature comprises these 12 or 14 genes plus additional genes. One skilled in the art can perform expression profiling of the 12 or 14 genes (or a subset thereof, or additional genes) described herein, using RNA obtained from a variety of possible sources, and then apply the expression data in one or more of the exemplary algorithms provided herein to determine a predictive or prognostic metastasis score (MS). For example, mRNA from a breast cancer patient's tumor sample can be obtained from formalin-fixed, paraffin-embedded (FFPE) tissue sections (or other samples such as tumor biopsy samples or frozen tumor tissue samples), and the mRNA expression levels of the 12 or 14 genes (or a subset thereof, or additional genes) can be measured to assess whether an individual will respond to chemotherapy for treating breast cancer.

In certain aspects of the invention, it relates to a method of determining whether a breast cancer patient will respond to (benefit from) a chemotherapy, the method comprising measuring mRNA expression of the genes known as CENPA, PKMYT1, MELK, MYBL2, BUB1, RACGAP1, TK1, UBE2S, DC13, RFC4, PRR11, DIAPH3, ORC6L and CCNB1 (or a subset thereof, such as any 5, 6, 7, 8, 9, 10, 11, 12, or 13 of these genes; particularly the 12 genes CENPA, PKMYT1, MELK, BUB1, RACGAP1, TK1, UBE2S, DC13, RFC4, PRR11, DIAPH3, and ORC6L; or these 12 or 14 genes plus additional genes) in tumor cells (particularly estrogen receptor-positive tumor cells) of the breast cancer patient, and determining whether the patient will respond to the chemotherapy based on mRNA expression levels of these genes. In exemplary embodiments, the breast cancer patient is determined to have a decreased likelihood of responding to chemotherapy due to increased (elevated) expression levels of the assayed genes, or the breast cancer patient is determined to have an increased likelihood of responding to chemotherapy due to decreased (e.g., normal or not increased) expression levels of the assayed genes. In certain exemplary aspects, the chemotherapy is CMF, CAF, AC, AC-Taxol®, or other chemotherapy. In certain embodiments, the method comprises determining whether a breast cancer patient will respond to chemotherapy that is given in conjunction with tamoxifen (e.g., determining whether a breast cancer patient should be treated with chemotherapy, such as CMF or CAF, prior to, concomitantly, or after, being treated with tamoxifen). Furthermore, in certain embodiments, the breast cancer patient is a postmenopausal woman.

In certain embodiments, methods are provided for determining risk of tumor metastasis in a breast cancer patient, comprising measuring the expression level of a subset (e.g., any 5, 6, 7, 8, 9, 10, 11, 12, or 13) of the genes CENPA, PKMYT1, MELK, MYBL2, BUB1, RACGAP1, TK1, UBE2S, DC13, RFC4, PRR11, DIAPH3, ORC6L and CCNB1 (particularly the 12 genes CENPA, PKMYT1, MELK, BUB1, RACGAP1, TK1, UBE2S, DC13, RFC4, PRR11, DIAPH3, and ORC6L; or these 12 genes plus additional genes) in tumor cells (particularly estrogen receptor-positive tumor cells) of said breast cancer patient, and determining risk of tumor metastasis based on mRNA expression levels of the subset of genes. In exemplary embodiments, the breast cancer patient is determined to have an increased risk for tumor metastasis due to increased (elevated) expression levels of the assayed subset of genes, or the breast cancer patient is determined to have a decreased risk for tumor metastasis due to decreased (e.g., normal or not increased) expression levels of the assayed subset of genes.

In other aspects of the invention, it relates to a method of determining whether a breast cancer patient will respond to (benefit from) a chemotherapy, the method comprising measuring the expression level of genes comprising CENPA, PKMYT1, MELK, MYBL2, BUB1, RACGAP1, TK1, UBE2S, DC13, RFC4, PRR11, DIAPH3, ORC6L and CCNB1 (or a subset thereof, such as any 5, 6, 7, 8, 9, 10, 11, 12, or 13 of these genes; particularly the 12 genes CENPA, PKMYT1, MELK, BUB1, RACGAP1, TK1, UBE2S, DC13, RFC4, PRR11, DIAPH3; or these 12 or 14 genes plus additional genes) in tumor cells (particularly estrogen receptor-positive tumor cells) of said breast cancer patient, thereby obtaining a metastasis score (MS) based upon the expression levels of these genes, and determining whether the patient will respond to the chemotherapy based on the MS, which can optionally be carried out by comparing the MS to one or more predefined metastasis score cut-points (MS thresholds). In exemplary embodiments, the breast cancer patient is determined to have a decreased likelihood of responding to chemotherapy due to a high MS (e.g., an MS that is at or above a predefined MS threshold), or the breast cancer patient is determined to have an increased likelihood of responding to chemotherapy due to a low MS (e.g., an MS that is below a predefined MS threshold). In certain embodiments, the method comprises determining whether a breast cancer patient will respond to a chemotherapy that is given in conjunction with tamoxifen (e.g., determining whether a breast cancer patient should be treated with chemotherapy, such as CMF or CAF, prior to, concomitantly, or after, being treated with tamoxifen). Furthermore, in certain embodiments, the breast cancer patient is a postmenopausal woman.

In certain embodiments, methods are provided for determining risk of tumor metastasis in a breast cancer patient, comprising measuring the expression level of a subset (e.g., any 5, 6, 7, 8, 9, 10, 11, 12, or 13) of the genes CENPA, PKMYT1, MELK, MYBL2, BUB1, RACGAP1, TK1, UBE2S, DC13, RFC4, PRR11, DIAPH3, ORC6L and CCNB1 (particularly the 12 genes CENPA, PKMYT1, MELK, BUB1, RACGAP1, TK1, UBE2S, DC13, RFC4, PRR11, DIAPH3, and ORC6L; or these 12 genes plus additional genes) in tumor cells (particularly estrogen receptor-positive tumor cells) of said breast cancer patient, thereby obtaining a metastasis score (MS) based upon the expression levels of the subset of genes, and determining whether the patient is at increased risk for metastasis based on the MS, which can optionally be carried out by comparing the MS to one or more predefined metastasis score cut-points (MS thresholds). In exemplary embodiments, the breast cancer patient is determined to have an increased risk for tumor metastasis due to a high MS for the subset of genes (e.g., an MS that is at or above a predefined MS threshold), or the breast cancer patient is determined to have a decreased risk for tumor metastasis due to a low MS for the subset of genes (e.g., an MS that is below a predefined MS threshold).

In a further aspect of the invention, the breast cancer patient is determined to have an increased risk of tumor metastasis and/or a decreased likelihood of responding to (benefiting from) chemotherapy (e.g., CAF or CMF chemotherapy), if their MS is higher than the predefined MS threshold.

In another aspect of the invention, the breast cancer patient is determined to have a decreased risk of tumor metastasis and/or an increased likelihood of responding to (benefiting from) chemotherapy (e.g., CAF or CMF chemotherapy), if their MS is lower than the predefined MS threshold.

In certain exemplary embodiments, methods are provided which further comprise assaying for (detecting) any of estrogen receptor (ER) (ESR1 gene), progesterone receptor (PR) (PGR gene), and/or HER2 (ERBB2 gene) status in conjunction with detecting gene expression of any of the 14 genes disclosed herein and/or in calculating an MS therefrom. For example, certain specific embodiments comprise detecting gene expression of all 14 genes of the MS, or a subset thereof (e.g., all 12 of the genes CENPA, PKMYT1, MELK, BUB1, RACGAP1, TK1, UBE2S, DC13, RFC4, PRR11, DIAPH3), optionally calculating an MS therefrom, and further assaying for any of ER, PR, and/or HER2 status (e.g., detection expression of any of the ESR1, PGR, and/or ERBB2 genes). For example, Table 31 shows exemplary multiplex assays configured to include reagents for detecting ESR1, PGR, and ERBB2.

In certain aspects of the invention, it relates to a method of determining chemotherapy response or risk of tumor metastasis in a breast cancer patient, in which mRNA of genes comprising the 14-gene signature (or a subset thereof) is obtained from breast tumor cells (e.g., ER-positive breast tumor cells), reverse transcribed to cDNA, and detected by polymerase chain reaction (PCR) amplification.

In other aspects of the invention, it relates to a method of determining chemotherapy response or risk of tumor metastasis in a breast cancer patient, in which mRNA of breast tumor cells (e.g., ER-positive breast tumor cells) is reverse transcribed and amplified by the two primers corresponding to each gene as presented in Table 3 (SEQ ID NOS:1-34).

In other aspects of the invention, it relates to a method of determining chemotherapy response or risk of tumor metastasis in a breast cancer patient, in which measurements of mRNA expression from ER-positive tumor cells are normalized against the mRNA expression of one or more control genes, such as one or more of the genes known as NUP214, PPIG and SLUT (see Table 2), or any combination thereof, which are examples of genes that can be used as endogenous control(s).

In another aspect of the invention, it relates to a method of determining chemotherapy response or risk of tumor metastasis in a breast cancer patient, in which mRNA expression from ER-positive tumor cells is detected by a nucleic acid array or real-time PCR.

In exemplary embodiments, the gene expression level (which can be represented by a Δ(ΔCt) value, such as calculated below in Equation 4) for each profiled gene is added together to obtain a score (referred to herein as a metastasis score (MS)) that represents the sum of the gene expression levels (e.g., the sum of the Δ(ΔCt) values) for all of the profiled genes. In exemplary embodiments, the gene expression levels (e.g., Δ(ΔCt) values) for each of the genes is equally weighted when they are added together to obtain the score, however in alternative embodiments, the gene expression levels (e.g., Δ(ΔCt) values) for each of the genes can be differentially weighted (e.g., each gene can be weighted by a particular constant value, such as the exemplary ai value provided for each gene in Table 2). Thus, in certain exemplary embodiments, the score (referred to herein as a MS) is the sum of the expression levels, as indicated by Δ(ΔCt) values, for all of the profiled genes (e.g., all 14 genes; or all 12 of the genes CENPA, PKMYT1, MELK, BUB1, RACGAP1, TK1, UBE2S, DC13, RFC4, PRR11, DIAPH3, and ORC6L; or any other subcombination of the 14 genes disclosed herein). In exemplary embodiments, this score (MS) is used to predict chemotherapy response and/or determine risk for tumor metastasis in a breast cancer patient. For example, the score (MS) can be compared against a predetermined threshold (also referred to as a cut-point or cutoff), such as to categorize a sample as having either a low or high score. In exemplary embodiments, a low score indicates that the individual would have an increased response to chemotherapy (i.e., they would benefit from chemotherapy treatment) and a decreased risk for tumor metastasis, whereas a high score indicates that the individual would have a decreased response to chemotherapy and an increased risk for tumor metastasis.

In certain aspects of the invention, it relates to a method of determining chemotherapy response and/or risk of tumor metastasis in a breast cancer patient, in which the mRNA expression is computed into a metastasis score (MS), such as by the following:

$$M S = a 0 + {\sum }_{i = 1}^{M} ai * Gi$$

where M=14 (in exemplary embodiments which utilize the 14 genes), Gi=the standardized expression level (e.g., a Δ(ΔCt) value) of each gene (i) of the 14 said genes (or subset thereof), a0=0.022 (as an example value), and ai can optionally correspond to the value presented in Table 2 for each of the genes in the 14-gene signature (or subset thereof) in embodiments in which each gene is differentially weighted (however, in other embodiments, each gene is equally weighted, in which instance ai=1). In certain embodiments, a subset of the 14 genes is used in computing the metastasis score (MS), such as wherein M=5, 6, 7, 8, 9, 10, 11, 12, or 13 (for example, M=12 in embodiments utilizing the 12 genes CENPA, PKMYT1, MELK, BUB1, RACGAP1, TK1, UBE2S, DC13, RFC4, PRR11, DIAPH3, and ORC6L). In certain embodiments, all of the 14 genes plus additional genes are used in computing the MS (in which instance M>14).

In other aspects of the invention, it relates to a method of determining chemotherapy response and/or risk of tumor metastasis in a breast cancer patient, in which the mRNA expression is computed into a metastasis score (MS) by the following:

$$M S = a 0 + b * [ {\sum }_{i = 1}^{M} ai * Gi ] \qquad Equation 1$$

where M=14 (in exemplary embodiments which utilize the 14 genes), Gi=the standardized expression level (e.g., a Δ(ΔCt) value) of each gene (i) of the 14 said genes (or subset thereof), a0=0.022 (as an example value), b=−0.251 (as an example value) and ai can optionally correspond to the value presented in Table 2 for each of the genes in the 14-gene signature (or subset thereof) in embodiments in which each gene is differentially weighted (however, in other embodiments, each gene is equally weighted, in which instance ai=1). Standardized expression level is obtained by subtracting the mean expression of that gene in the training set from the expression level measured in Δ(ΔCt) and then divided by the standard deviation of the gene expression in that gene. The mean and standard deviation of gene expression for each gene in the training set are presented in Table 4. Equation 1 was used in Examples 1, 2 and 3. In certain embodiments, a subset of the 14 genes is used in computing the metastasis score (MS), such as wherein M=5, 6, 7, 8, 9, 10, 11, 12, or 13 (for example, M=12 in embodiments utilizing the 12 genes CENPA, PKMYT1, MELK, BUB1, RACGAP1, TK1, UBE2S, DC13, RFC4, PRR11, DIAPH3, and ORC6L). In certain embodiments, all of the 14 genes plus additional genes are used in computing the MS (in which instance M>14).

In further aspects of the invention, the MS formula can have the following definition:

$$M S = a 0 + b * [ {\sum }_{i = 1}^{M} ai * Gi ] \qquad Equation 2$$

where M=14 (in exemplary embodiments which utilize the 14 genes), Gi=expression level (e.g., a Δ(ΔCt) value) of each gene (i) of the 14 said genes (or subset thereof), a0=0.8657 (as an example value), b=−0.04778 (as an example value), ai=1 for all genes (in embodiments in which the genes are all weighted equally rather than being differentially weighted). Equation 2 was used in Examples 4 and 5. In certain embodiments, a subset of the 14 genes is used in computing the MS, such as wherein M=5, 6, 7, 8, 9, 10, 11, 12, or 13 (for example, M=12 in embodiments utilizing the 12 genes CENPA, PKMYT1, MELK, BUB1, RACGAP1, TK1, UBE2S, DC13, RFC4, PRR11, DIAPH3, and ORC6L). In certain embodiments, all of the 14 genes plus additional genes are used in computing the MS (in which instance M>14).

In a further aspect of the invention, the MS formula can have a0=0, b=−1, and/or ai=1.

Thus, in certain embodiments, the expression of each of the genes used in computing a MS (whether for determining chemotherapy response and/or risk for tumor metastasis) are all equally weighted. For example, in certain embodiments, gene expression of all 12 of the genes CENPA, PKMYT1, MELK, BUB1, RACGAP1, TK1, UBE2S, DC13, RFC4, PRR11, DIAPH3, and ORC6L (and optionally additional genes) is detected and weighted equally to determine response to chemotherapy and/or risk for tumor metastasis.

In other aspects of the invention, it relates to a method of determining chemotherapy response and/or risk of tumor metastasis in a breast cancer patient using expression profiling of the 14 genes in Table 2 (or a subset thereof, such as the 12 genes CENPA, PKMYT1, MELK, BUB1, RACGAP1, TK1, UBE2S, DC13, RFC4, PRR11, DIAPH3, and ORC6L), in which the expression level Gi of each gene (i) is computed into a gene expression value Gi by the following:

Δ(ΔCt)=(Ct_{GOI}−Ct_{EC})_{test RNA}−(Ct_{GOI}−Ct_{EC})_{ref RNA} Equation 4

where Ct is the threshold cycle for target amplification, GOI=gene of interest, EC=endogenous control, test RNA=patient sample RNA, ref RNA=reference RNA.

In other aspects of the invention, it relates to a method of determining chemotherapy response and/or risk of tumor metastasis in a breast cancer patient using expression profiling of the 14 genes in Table 2 (or a subset thereof), in which the expression level Gi of each gene (i) is combined into a single value of MS score, wherein a patient with a MS score higher than the relevant (e.g., predetermined) MS threshold or cut-point would be identified as having a decreased likelihood of responding to a chemotherapy (e.g., CMF or CAF chemotherapy) and/or as being at a higher risk for tumor metastasis, or wherein a patient with a MS score lower than the relevant MS threshold or cut-point would be identified as having an increased likelihood of responding to a chemotherapy (e.g., CMF or CAF chemotherapy) and/or as being at a lower risk for tumor metastasis.

Thus, in certain embodiments, a low MS (e.g., an MS below a certain MS threshold or cut-point) indicates that an individual has an increased likelihood of responding to (benefitting from) chemotherapy, whereas a high MS (e.g., an MS above a certain MS threshold or cut-point) indicates that an individual has a decreased likelihood of responding to chemotherapy.

In certain aspects of the invention, it relates to a kit comprising reagents for the detection of the expression levels of genes comprising CENPA, PKMYT1, MELK, MYBL2, BUB1, RACGAP1, TK1, UBE2S, DC13, RFC4, PRR11, DIAPH3, ORC6L and CCNB1, an enzyme; and a buffer. In certain embodiments, the kit comprises reagents for the detection of the expression levels of a subset of these 14 genes, such as any 5, 6, 7, 8, 9, 10, 11, 12, or 13 of these genes (particularly the 12 genes CENPA, PKMYT1, MELK, BUB1, RACGAP1, TK1, UBE2S, DC13, RFC4, PRR11, DIAPH3, and ORC6L). In certain embodiments, the kit comprises reagents for detecting the expression levels of these 14 genes (or a subset thereof, such as the aforementioned 12 genes) plus additional genes. In particular embodiments, the kit is specifically for use in determining chemotherapy response and/or risk of tumor metastasis in a breast cancer patient, and can optionally include instructions therefor. The kit can also optionally include instructions for generating a score (e.g., an MS) from the expression levels of the genes and can optionally further include instructions for evaluating the score (e.g., comparing the score to a predetermined threshold in order to categorize a sample, such as with respect to chemotherapy response and/or metastasis risk).

In other aspects of the invention, it relates to a nucleic acid array comprising polynucleotides that hybridize to genes comprising CENPA, PKMYT1, MELK, MYBL2, BUB1, RACGAP1, TK1, UBE2S, DC13, RFC4, PRR11, DIAPH3, ORC6L and CCNB1. In certain embodiments, the array comprises polynucleotides that hybridize to a subset of these 14 genes, such as any 5, 6, 7, 8, 9, 10, 11, 12, or 13 of these genes (particularly the 12 genes CENPA, PKMYT1, MELK, BUB1, RACGAP1, TK1, UBE2S, DC13, RFC4, PRR11, DIAPH3, and ORC6L). In certain embodiments, the array comprises polynucleotides that hybridize to these 14 genes (or a subset thereof, such as the aforementioned 12 genes) plus additional genes. In particular embodiments, the nucleic acid array is specifically for use in determining chemotherapy response and/or risk of tumor metastasis in a breast cancer patient.

### SEQUENCE LISTING

A Sequence Listing is provided as part of the instant application. The Sequence Listing provides the oligonucleotide sequences (SEQ ID NOS:1-34) as shown in Table 3. These oligonucleotides are exemplary primers that can be used for, for example, RT-PCR amplification of the genes listed in Table 3.

### BRIEF DESCRIPTIONS OF THE FIGURES

FIG. 1 shows Kaplan-Meier curves for a) time to distant metastases b) overall survival for training set from CPMC where high-risk and low-risk groups were defined by a “cross-validated” metastasis score (referred to herein as “MS (CV)”) using zero as cut point (see Example One below).

FIG. 2 is a ROC curve for predicting distant metastases by MS (CV) in 5 years in the training set from CPMC. AUC=0.76 (0.65-0.87).

FIG. 3 shows Kaplan-Meier curves by risk groups defined by the gene signature and Adjuvant! in 280 untreated patients from Guy's Hospital. Specifically, FIGS. 3 a) and b) describe results using the 14-gene signature, and FIGS. 3 c) and d) describe results using the Adjuvant! factors. a) Time to distant metastases (DMFS) by MS risk groups b) Overall survival by MS risk groups c) Time to distant metastases (DMFS) by Adjuvant! risk groups d) Overall survival by Adjuvant! risk groups.

FIG. 4 shows Receiver operating characteristic (ROC) curves of the 14-gene signature and of the online program Adjuvant! a) ROC curve for distant metastases within 5 years for the 14-gene signature b) ROC curve for distant metastases within 10 years for the 14-gene signature c) ROC curve for death within 10 years for the 14-gene signature d) ROC curve for metastases within 5 years for Adjuvant! e) ROC curve for metastases within 10 years for Adjuvant! f) ROC curve for death within 10 years for Adjuvant! for untreated patients from Guy's Hospital

FIG. 5 shows probability of distant metastasis within 5 years and 10 years vs. metastasis score (MS) from 280 Guy's untreated patients.

FIG. 6 is a comparison of probability of distant metastasis in 10 years from the 14-gene signature vs, 10-year relapse probability from Adjuvant! for untreated patients from Guy's Hospital

FIG. 7 shows Kaplan-Meier curves for distant-metastasis-free survival in University of Muenster patients.

FIG. 8 shows Kaplan-Meier curves of distant-metastasis-free survival in 3 MS groups (high, intermediate, low) for 205 treated patients from Guy's Hospital.

FIG. 9 shows Kaplan-Meier curves of distant-metastasis-free survival in 2 risk groups (high and low) determined by MS for 205 treated patients from Guy's Hospital.

FIG. 10 shows ROC curve of MS to predict distant metastasis in 5 years for Guy's treated samples, AUC=0.7 (0.57-0.87).

FIG. 11 shows time dependence of hazard ratios of high vs. low risk groups by MS in Guy's treated samples.

FIG. 12 shows Kaplan-Meier curves of distant-metastasis-free survival (DMFS) for three MS groups (high, intermediate and low) in 234 Japanese samples.

FIG. 13 shows Kaplan-Meier curves of distant-metastasis-free survival (DMFS) for two risk groups (high MS have high risk whereas intermediate and low MS have low risk) in 234 Japanese samples.

FIG. 14 shows ROC curve of MS to predict distant metastasis in 5 years for Japanese patients. AUC=0.73 (0.63-0.84).

FIG. 15 shows annualized hazard rate for MS groups and hazard ratio of high vs. low risk groups as a function of time.

FIG. 16 shows Kaplan-Meier (KM) plots for 7-year disease-free survival (DFS) by treatment for MS High and Low classifications of Trial IX. FIG. 16 demonstrates that low MS is predictive of chemotherapy response (i.e., chemotherapy benefit).

FIG. 17 shows STEPP Plots for MS and Ki-67 in Trial IX.

FIG. 18 shows a comparison of Kaplan Meier plots for metastasis scores based on the 14-gene signature (“MS 14”) vs. a 12-gene signature (“MS 12”) for predictive use based on an analysis of Trial IX samples (the 12-gene signature differs from the 14-gene signature in that MYBL12 and CCNB1 were excluded).

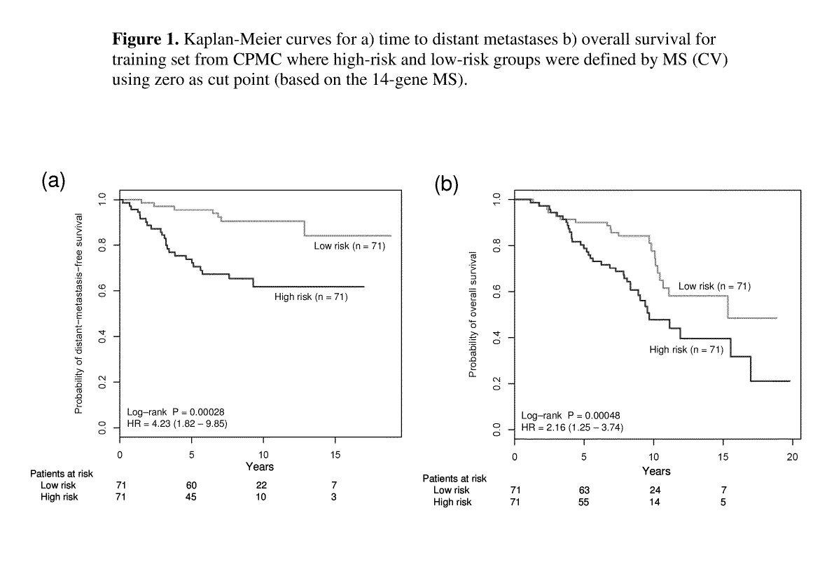

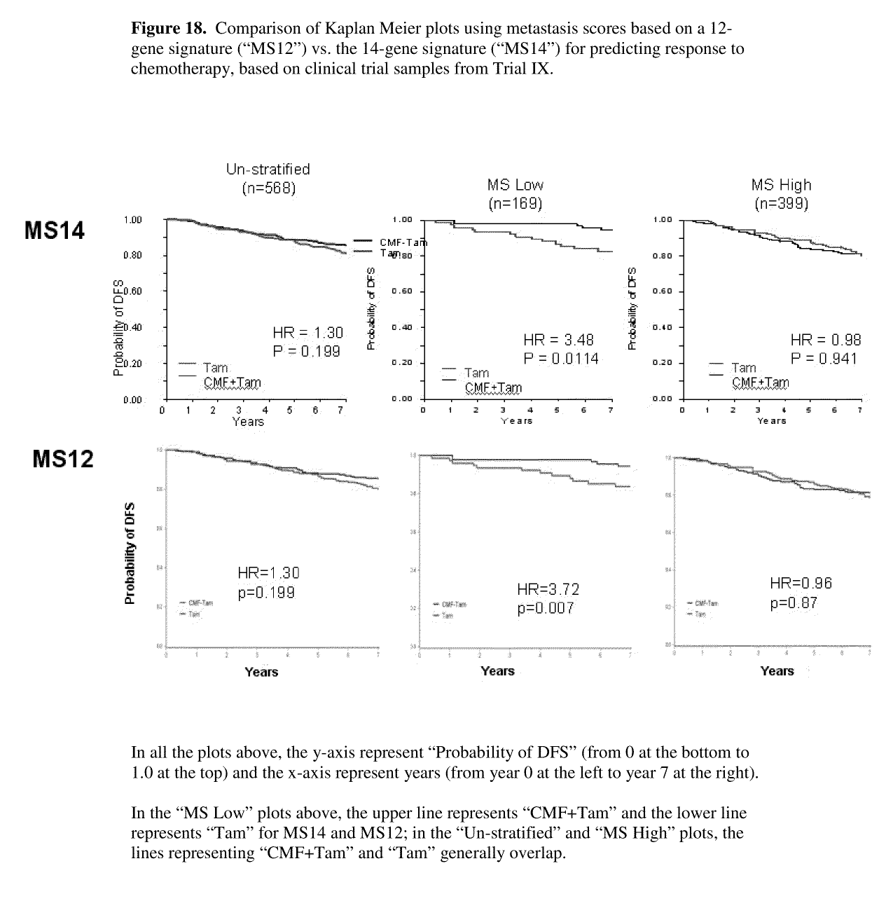

FIGS. 1-17 are based on the 14-gene MS (CENPA, PKMYT1, MELK, MYBL2, BUB1, RACGAP1, TK1, UBE2S, DC13, RFC4, PRR11, DIAPH3, ORC6L and CCNB1), and FIG. 18 is a comparison of this 14-gene MS vs. the 12-gene MS (CENPA, PKMYT1, MELK, BUB1, RACGAP1, TK1, UBE2S, DC13, RFC4, PRR11, DIAPH3, and ORC6L).

### BRIEF DESCRIPTION OF TABLE 40

Table 40 provides correlation coefficients between all possible subsets of the 14 genes with the full set of 14 genes described herein (and provided in Table 2) for determining response to chemotherapy (based on data from IBCSG Trial IX, which is described below in Example Six), as well as for determining risk for breast tumor metastasis. Table 40 provides three types of correlation coefficients for each subset of genes: Pearson, Spearman, and Kendall's Tau correlation coefficients (see Example Seven below). These correlation coefficients indicate how closely results derived from gene expression analysis of each subset of the 14 genes would correlate with results derived from gene expression analysis of the full set of 14 genes for predicting response to chemotherapy or determining risk for breast tumor metastasis.

### DETAILED DESCRIPTION OF EXEMPLARY EMBODIMENTS OF THE INVENTION

Exemplary embodiments of the invention provide multi-gene signatures and methods of using the multi-gene signatures for predicting response to breast cancer chemotherapy (e.g., CMF, CAF, AC, AC-Taxol®, or other chemotherapy) or for determining risk of breast cancer metastasis, methods and reagents for the detection of the genes disclosed herein, and assays or kits that utilize such reagents. The breast cancer-associated genes disclosed herein are useful for determining whether an individual with breast cancer (such as a postmenopausal woman) will respond to chemotherapy, as well as for diagnosing, screening for, and evaluating probability of distant metastasis of ER-positive tumors in breast cancer patients.

Expression profiling of the 14 genes (or a subset thereof, such as an exemplary 12-gene signature described herein, or the 12 or 14 genes plus additional genes) of the multi-gene signature disclosed in Table 2 enables a prediction or determination of chemotherapy response or a prognosis of distant metastasis to be determined. The information provided in Table 2 includes a reference sequence (RefSeq), obtained from the National Center for Biotechnology Information (NCBI) of the National Institutes of Health/National Library of Medicine, which identifies one exemplary variant transcript sequence of each described gene. Based on the sequence of the variant, reagents may be designed to detect all variants of each gene of the 14-gene signature (or subset thereof). Table 3 provides exemplary primer pairs that can be used to detect each gene of the 14-gene signature in a manner such that all variants of each gene are amplified. Thus, various embodiments of the invention provide for expression profiling of all known transcript variants of each of the genes disclosed herein.

Also shown in Table 2 is a reference that published the nucleotide sequence of each RefSeq. These references are all herein incorporated by reference in their entirety. Also in Table 2 is a description of each gene. The references and descriptions provided in Table 2 are from NCBI.

The CENPA gene is identified by reference sequence NM_{—}001809 and disclosed in Black, B. E., Foltz, D. R., et al., 2004, Nature 430(6999):578-582. Said reference sequence and reference are herein incorporated by reference in their entirety.

The PKMYT1 gene, identified by reference sequence NM_{—}004203, and disclosed in Bryan, B. A., Dyson, O. F. et al., 2006, J. Gen. Virol. 87 (PT 3), 519-529. Said reference sequence and reference are herein incorporated by reference in their entirety.

The MELK gene, identified by reference sequence NM 014791, and disclosed in Beullens, M., Vancauwenbergh, S. et al., 2005, J. Biol. Chem. 280 (48), 40003-40011. Said reference sequence and reference are herein incorporated by reference in their entirety.

The MYBL2 gene, identified by reference sequence NM_{—}002466, and disclosed in Bryan, B. A., Dyson, O. F. et al., 2006, J. Gen. Virol. 87 (PT 3), 519-529. Said reference sequence and reference are herein incorporated by reference in their entirety.

The BUB1 gene, identified by reference sequence NM_{—}004336, and disclosed in Morrow, C. J., Tighe, A. et al., 2005, J. Cell. Sci. 118 (PT 16), 3639-3652. Said reference sequence and reference are herein incorporated by reference in their entirety.

The RACGAP1 gene, identified by reference sequence NM_{—}013277, and disclosed in Niiya, F., Xie, X. et al., 2005, J. Biol. Chem. 280 (43), 36502-36509. Said reference sequence and reference are herein incorporated by reference in their entirety.

The TK1 gene, identified by reference sequence NM_{—}003258, and disclosed in Karbownik, M., Brzezianska, E. et al., 2005, Cancer Lett. 225 (2), 267-273. Said reference sequence and reference are herein incorporated by reference in their entirety.

The UBE2S gene, identified by reference sequence NM_{—}014501, and disclosed in Liu, Z., Diaz, L. A. et al., 1992, J. Biol. Chem. 267 (22), 15829-15835. Said reference sequence and reference are herein incorporated by reference in their entirety.

The DC13 gene, identified by reference sequence AF201935, and disclosed in Gu, Y., Peng, Y. et al., Direct Submission, Submitted Nov. 5, 1999, Chinese National Human Genome Center at Shanghai, 351 Guo Shoujing Road, Zhangjiang Hi-Tech Park, Pudong, Shanghai 201203, P. R. China. Said reference sequence and reference are herein incorporated by reference in their entirety.

The RFC4 gene, identified by reference sequence NM 002916, and disclosed in Gupte, R. S., Weng, Y. et al., 2005, Cell Cycle 4 (2), 323-329. Said reference sequence and reference are herein incorporated by reference in their entirety.

The PRR11 gene, identified by reference sequence NM_{—}018304, and disclosed in Weinmann, A. S., Yan, P. S. et al., 2002, Genes Dev. 16 (2), 235-244. Said reference sequence and reference are herein incorporated by reference in their entirety.

The DIAPH3 gene, identified by reference sequence NM 030932, and disclosed in Katoh, M. and Katoh, M., 2004, Int. J. Mol. Med. 13 (3), 473-478. Said reference sequence and reference are herein incorporated by reference in their entirety.

The ORC6L gene, identified by reference sequence NM_{—}014321, and disclosed in Sibani, S., Price, G. B. et al., 2005, Biochemistry 44 (21), 7885-7896. Said reference sequence and reference are herein incorporated by reference in their entirety.

The CCNB1 gene, identified by reference sequence NM_{—}031966, and disclosed in Zhao, M., Kim, Y. T. et al., 2006, Exp Oncol 28 (1), 44-48. Said reference sequence and reference are herein incorporated by reference in their entirety.

The PPIG gene, identified by reference sequence NM_{—}004792, and disclosed in Lin, C. L., Leu, S. et al., 2004, Biochem. Biophys. Res. Commun. 321 (3), 638-647. Said reference sequence and reference are herein incorporated by reference in their entirety.

The NUP214 gene, identified by reference sequence NM_{—}005085, and disclosed in Graux, C., Cools, J. et al., 2004, Nat. Genet. 36 (10), 1084-1089. Said reference sequence and reference are herein incorporated by reference in their entirety.

The SLUT gene, identified by reference sequence NM_{—}006425, and disclosed in Shomron, N., Alberstein, M. et al., 2005, J. Cell. Sci. 118 (PT 6), 1151-1159. Said reference sequence and reference are herein incorporated by reference in their entirety.

Also shown in Table 2 is an exemplary value for the constant ai that can be used for determining the metastasis score (MS) for each gene i, based on the expression profiling results obtained for that gene (however, alternative ai values can be used instead of the exemplary ai values provided in Table 2 for any of the genes; furthermore, the expression of each of the genes used in computing the MS, whether for determining chemotherapy response and/or risk for tumor metastasis, can all be equally weighted (e.g., ai=1 for all genes)). Exemplary methods of deriving a metastasis score (MS) and applying/using an MS, and methods of gene expression profiling and use of the data obtained therefrom, are described below.

Thus, exemplary embodiments of the invention provide 14 individual genes (Table 2) (including subsets of these 14 genes; particularly the 12 genes CENPA, PKMYT1, MELK, BUB1, RACGAP1, TK1, UBE2S, DC13, RFC4, PRR11, DIAPH3, and ORC6L; as well as these 12 or 14 genes plus additional genes) which together are predictive for breast cancer chemotherapy response and/or prognostic for risk of breast cancer metastasis, methods of determining expression levels of these genes in a test sample, methods of determining the probability of an individual of responding to a chemotherapy and/or developing distant metastasis, methods of generating a metastasis score (MS) (which may also be referred to herein as a “score”) based on the expression levels of these genes, and methods of using the disclosed genes to select a chemotherapeutic treatment regimen.

The present invention provides a unique combination (which may be interchangeably referred to herein as a “signature”, “molecular signature”, “expression signature”, “prognostic signature”, “predictive signature”, “combination”, or “panel”) of 14 genes, as well as subsets (which may alternatively be referred to herein as subcombinations) of these 14 genes, such as subsets of any 5, 6, 7, 8, 9, 10, 11, 12, or 13 of these 14 genes (particularly a subset comprising, or consisting of, all 12 of the genes CENPA, PKMYT1, MELK, BUB1, RACGAP1, TK1, UBE2S, DC13, RFC4, PRR11, DIAPH3, and ORC6L), as well as combinations comprising these 12 (or other subset) or 14 genes plus additional genes. Exemplary subsets are provided in Table 40. Accordingly, in exemplary embodiments, the invention provides novel methods based on the unique combinations of genes disclosed herein, such as methods relating to predicting chemotherapy response (e.g., response to CMF, CAF, AC, AC-Taxol®, or other chemotherapy) or determining metastasis risk in a breast cancer patient.

In certain exemplary embodiments, the chemotherapy comprises at least one of an anthracycline (e.g., doxorubicin and/or epirubicin), a taxane (e.g., paclitaxel and/or docetaxel), an inhibitor of nucleotide synthesis (e.g., methotrexate and/or 5-fluorouracil (5-FU)), and/or an alkylating agent (e.g., cyclophosphamide).

In certain exemplary embodiments, the chemotherapy comprises CMF (cyclophosphamide, methotrexate, fluorouracil (5-FU)), CAF (cyclophosphamide, Adriamycin® (doxorubicin), fluorouracil (5-FU)), AC (also known as CA) [Adriamycin® (doxorubicin) and cyclophosphamide] or AC-Taxol® (AC followed by paclitaxel), as well as other chemotherapy regimens comprising any one or more of the individual chemotherapeutic agents which make-up either of the aforementioned chemotherapy regimens. In certain embodiments, a breast cancer patient is treated with chemotherapy in addition to tamoxifen (e.g., chemotherapy treatment precedes tamoxifen treatment of an individual with breast cancer, or chemotherapy is administered after tamoxifen treatment).

In further exemplary embodiments, the chemotherapy can comprise another chemotherapy regimen other than CMF, CAF, AC, or AC-Taxol®, such as, for example: TAC [Taxotere® (docetaxel), Adriamycin® (doxorubicin), and cyclophosphamide)], EC (epirubicin and cyclophosphamide), FEC (5-fluorouracil, epirubicin and cyclophosphamide), FECD (FEC followed by docetaxel), TC [Taxotere® (docetaxel) and cyclophosphamide], or MF (methotrexate and fluorouracil (5-FU)), as well as other chemotherapy regimens comprising any one or more of the individual chemotherapeutic agents which make up any of the aforementioned chemotherapy regimens. Any chemotherapy regimens can optionally include paclitaxel (e.g., Taxol® or Abraxane®) and/or docetaxel (e.g., Taxotere®), beyond those mentioned above. Furthermore, any chemotherapy regimens (including CMF and CAF) can also optionally further include one or more non-chemotherapy agents such as trastuzumab (Herceptin®) in combination with the chemotherapy.

Exemplary embodiments of the invention provide methods for predicting response to chemotherapy in an individual who has breast cancer, particularly wherein the individual is a postmenopausal woman. Certain embodiments of the invention provide methods for predicting risk of metastasis, particularly distant metastasis, in an individual who has breast cancer. In certain embodiments, the breast tumor is estrogen receptor (ER)-positive and, in certain embodiments, the breast tumor is HER2-negative. Furthermore, in certain embodiments, the individual's breast cancer is lymph node-negative.

In certain exemplary embodiments of the invention, the response to breast cancer chemotherapy as predicted by gene expression analysis of the 14-gene signature (or a subset thereof, such as the 12-gene combination of CENPA, PKMYT1, MELK, BUB1, RACGAP1, TK1, UBE2S, DC13, RFC4, PRR11, DIAPH3, and ORC6L, or the 12 or 14 genes plus additional genes), such as by determining a metastasis score (MS), is negatively (inversely) correlated with risk for breast cancer metastasis as predicted by the MS (or by gene expression analysis without deriving an MS), particularly in instances wherein the subject is a postmenopausal woman. Thus, in certain embodiments, an individual with breast cancer who is predicted to be at increased risk for tumor metastasis based on their MS (or gene expression analysis without deriving an MS) is also predicted to be less likely to respond to (i.e., benefit from) chemotherapy (e.g., CMF, CAF, AC, AC-Taxol®, or other chemotherapy). Alternatively, an individual with breast cancer who is predicted to be at less risk for tumor metastasis based on their MS (or gene expression analysis without deriving an MS) is also predicted to be more likely to respond to chemotherapy (e.g., CMF, CAF, AC, AC-Taxol®, or other chemotherapy). Thus, in certain embodiments, a low MS (or gene expression which is not elevated) indicates an increased likelihood of responding to chemotherapy (and a decreased risk for metastasis), whereas a high MS (or elevated gene expression) indicates a decreased likelihood of responding to chemotherapy (and an increased risk for metastasis) for a breast cancer patient (particularly a postmenopausal patient).

Similarly, in certain exemplary embodiments, the response to breast cancer chemotherapy as predicted by gene expression analysis of any one or more of the genes of the 14-gene signature (or a subset thereof) is negatively (inversely) correlated with risk for breast cancer metastasis as predicted by expression of that gene, particularly in instances wherein the patient is a postmenopausal woman. For example, in certain embodiments, the response to breast cancer chemotherapy as predicted by the expression of the MYBL2 gene is negatively (inversely) correlated with risk for breast cancer metastasis as predicted by expression of MYBL2, particularly in instances wherein the subject is a postmenopausal woman.

Prediction of chemotherapy response based on gene expression analysis of the 14-gene signature (or a subset thereof, such as the exemplary 12-gene signature disclosed herein, or the 12 or 14 genes plus additional genes) can be independent of any assessment of risk. Thus, it is not necessary that prediction of chemotherapy response be coupled with a determination of metastasis risk. Determination of risk for breast cancer metastasis does not necessarily need to be done at all in order to determine response to chemotherapy. Rather, gene expression analysis of the 14 genes (or a subset thereof, such as the exemplary 12-gene signature disclosed herein, or the 12 or 14 genes plus additional genes) can be carried out solely for the purpose of predicting response to chemotherapy. For example, in certain embodiments, a metastasis score (MS) can be derived from gene expression analysis of the 14 genes (or a subset thereof, such as the exemplary 12-gene signature disclosed herein, or the 12 or 14 genes plus additional genes) of a breast cancer patient, and a low MS value (e.g., an MS value that is below a certain cutoff/threshold value) indicates that the breast cancer patient has an increased likelihood of responding to (benefitting from) chemotherapy, whereas a high MS value (e.g., an MS value that is above a certain cutoff/threshold value) indicates that the breast cancer patient has a decreased likelihood of responding to (benefitting from) chemotherapy. Embodiments such as these do not require any inquiry into metastasis risk (although determination of metastasis risk can also optionally be included as part of the analysis).

Examples of MS threshold values (which may also be interchangeably referred to herein as cutoffs, cutpoints, thresholds, or threshold scores) which could be used, such as to distinguish “high” versus “low” MS classifications, include 1.738, 1.74, 1.75, etc. (based on an un-scaled MS), however other alternative values could be used as well. Furthermore, the MS can optionally be re-scaled (e.g., re-scaled to a 0-40 or other scale), in which instance correspondingly re-scaled MS threshold values can be used, such as 17.38, 17.4, and 17.5, respectively (based on an MS that is re-scaled to a 0-40 scale). As just one example, based on an MS threshold value of 17.5, an MS<17.5 can be classified as a “low” MS (e.g., this would identify those individuals, particularly postmenopausal women, who would be predicted to respond to chemotherapy and/or who are at lower risk for breast cancer metastasis), whereas an MS>17.5 can be classified as a “high” MS (e.g., this would identify those individuals, particularly postmenopausal women, who would be predicted to not respond to chemotherapy and/or who are at increased risk for breast cancer metastasis). As another example, an MS threshold value (cut-point) of 15 can be utilized (particularly in embodiments which utilize the 12 genes CENPA, PKMYT1, MELK, BUB1, RACGAP1, TK1, UBE2S, DC13, RFC4, PRR11, DIAPH3, and ORC6L), such as to classify a sample as either low MS or high MS (such as for predicting chemotherapy response and/or risk for tumor metastasis). When the MS is used for both predicting response to chemotherapy and for determining metastasis risk, either the same MS threshold value can be used for both predicting response to chemotherapy and for determining risk for metastasis (as in the above example using a 17.5 MS threshold value to both predict chemotherapy response and to determine metastasis risk) or, alternatively, the MS threshold value that is used for predicting response to chemotherapy can be a different value than the MS threshold value that is used for determining risk for metastasis. Furthermore, MS threshold values can be adjusted if different subsets of the 14 genes are utilized (such as those provided in Table 40).

Furthermore, rather than using a discrete cutoff (threshold) for discrete categorization of a sample, a continuous analysis can be employed instead (such as exemplified in Table 34 and Table 35, and Example Six). Thus, an MS can be applied in a categorical or continuous manner to determine and/or characterize (e.g., report) an individual's predicted chemotherapy response and/or risk for metastasis.

As indicated above, the MS can optionally be re-scaled (and the MS threshold can also optionally be re-scaled accordingly). Examples of re-scaling the MS is described in, for example, U.S. Pat. No. 8,557,525 (Wang et al., “A Composite Metastasis Score with Weighted Coefficients”, filed Feb. 27, 2012, issued Oct. 15, 2013), which is incorporated herein by reference. For example, the MS could be re-scaled to a 0-40 scale by shifting the scores by 60 and dividing by 2, as indicated by the following expression (where MSᵤ indicates an un-scaled MS):

MS=(MSᵤ+60)/2

where
MS=0 if MS<0
MS=40 if MS>40

Calculating a metastasis score (MS) is an optional step in the methods disclosed herein, and response to chemotherapy and/or risk for metastasis can be determined based on the gene expression of the 14 genes disclosed herein (and subsets thereof) without necessarily calculating an MS. For example, increased risk for breast cancer metastasis and/or decreased likelihood of responding to breast cancer chemotherapy can be determined based on increased (elevated) gene expression levels of the genes disclosed herein without necessarily calculating an MS, and decreased risk for breast cancer metastasis and/or increased likelihood of responding to breast cancer chemotherapy can be determined based on decreased (e.g., normal or not increased) gene expression levels of the genes disclosed herein without necessarily calculating an MS.

Those skilled in the art will readily recognize that nucleic acid molecules may be double-stranded molecules and that reference to a particular sequence of one strand refers, as well, to the corresponding site on a complementary strand. In defining a nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference may be made to either strand in order to refer to a particular nucleotide sequence. Probes and primers may be designed to hybridize to either strand and gene expression profiling methods disclosed herein may target either strand.

Subsets of the 14 Genes

In exemplary embodiments, the multi-gene signature comprises, or consists of, the following 14 genes: CENPA, PKMYT1, MELK, MYBL2, BUB1, RACGAP1, TK1, UBE2S, DC13, RFC4, PRR11 (also known as FLJ11029), DIAPH3, ORC6L, and CCNB1. In further exemplary embodiments, the multi-gene signature comprises, or consists of, a subset (subcombination) of these 14 genes. In particular, in various exemplary embodiments, the multi-gene signature comprises, or consists of, a subset of these 14 genes as set forth in Table 40. Table 40 provides correlation coefficients for each of the subsets which indicate how closely results derived from gene expression analysis of each subset of the 14 genes would correlate with results derived from gene expression analysis of the full set of 14 genes for determining response to chemotherapy, as well as for determining risk for metastasis (also see Example Seven below).

As an example, certain exemplary embodiments provide a multi-gene signature that comprises, or consists of, 13 out of the 14 genes. For example, the multi-gene signature can comprise, or consist of, all of the 14 genes except for CENPA, all of the 14 genes except for PKMYT1, all of the 14 genes except for MELK, all of the 14 genes except for MYBL2, all of the 14 genes except for BUB1, all of the 14 genes except for RACGAP1, all of the 14 genes except for TK1, all of the 14 genes except for UBE2S, all of the 14 genes except for DC13, all of the 14 genes except for RFC4, all of the 14 genes except for PRR11, all of the 14 genes except for DIAPH3, all of the 14 genes except for ORC6L, or all of the 14 genes except for CCNB1.

Similarly, in further exemplary embodiments, the multi-gene signature can comprise, or consist of, all of the 14 genes except for 2 of the genes (i.e., a 12-gene signature), all of the 14 genes except for 3 of the genes (i.e., an 11-gene signature), all of the 14 genes except for 4 of the genes (i.e., a 10-gene signature), all of the 14 genes except for 5 of the genes (i.e., a 9-gene signature), all of the 14 genes except for 6 of the genes (i.e., an 8-gene signature), all of the 14 genes except for 7 of the genes (i.e., a 7-gene signature), all of the 14 genes except for 8 of the genes (i.e., a 6-gene signature), or all of the 14 genes except for 9 of the genes (i.e., a 5-gene signature). Thus, in various exemplary embodiments, the multi-gene signature comprises, or consists of, any 5, 6, 7, 8, 9, 10, 11, 12, or 13 of the 14 genes (i.e., any combination of 5, 6, 7, 8, 9, 10, 11, 12, or 13 genes selected from the group consisting of CENPA, PKMYT1, MELK, MYBL2, BUB1, RACGAP1, TK1, UBE2S, DC13, RFC4, PRR11, DIAPH3, ORC6L, and CCNB1).

As just one example, an exemplary 12-gene signature can comprise, or consist of, all of the 14 genes except for MYBL2 and CCNB1 (i.e., a 12-gene signature which comprises, or consists of, all of the genes CENPA, PKMYT1, MELK, BUB1, RACGAP1, TK1, UBE2S, DC13, RFC4, PRR11, DIAPH3, and ORC6L) (which may be referred to herein as the “12-gene signature”, and is shown in bold on page 2 of Table 40). Thus, in particular exemplary embodiments, gene expression of a combination of genes comprising or consisting of all 12 of the genes CENPA, PKMYT1, MELK, BUB1, RACGAP1, TK1, UBE2S, DC13, RFC4, PRR11, DIAPH3, and ORC6L is detected, particularly to determine chemotherapy response and/or risk of tumor metastasis. A metastasis score (MS) can optionally be further calculated from the expression levels of these 12 genes, and this MS can be used to determine chemotherapy response and/or risk of tumor metastasis.

In addition to MYBL2 and/or CCNB1 being excluded from the multi-gene signature in certain embodiments, in various other exemplary embodiments of the multi-gene signature such genes as PKMYT1 and/or BUB1 can be excluded.

### Tumor Tissue Source and RNA Extraction

Exemplary embodiments of the invention can include extracting target polynucleotide molecules from a sample taken from an individual afflicted with breast cancer. The sample may be collected in any clinically acceptable manner such that gene-specific polynucleotides (e.g., transcript mRNA molecules) are preserved. The mRNA or other nucleic acids so obtained from the sample may then be analyzed further. For example, pairs of oligonucleotides specific for a gene (e.g., the genes presented in Table 2) may be used to amplify the specific mRNA(s) in the sample. The amount of mRNA can then be determined, or profiled, and the correlation with a disease prognosis can be made. Alternatively, mRNA or other nucleic acids derived therefrom (e.g., cDNA, amplified DNA, or enriched RNA) may be labeled distinguishably from standard or control polynucleotide molecules, and both may be simultaneously or independently hybridized to a nucleic acid array comprising some or all of the markers or marker sets or subsets described herein. Alternatively, mRNA or nucleic acids derived therefrom may be labeled with the same label as the standard or control polynucleotide molecules, wherein the intensity of hybridization of each at a particular probe is compared.

A sample may comprise any clinically relevant tissue sample, such as a formalin-fixed paraffin-embedded (FFPE) sample, frozen sample, tumor biopsy or fine needle aspirate, or a sample of bodily fluid containing ER-positive tumor cells such as blood, plasma, serum, lymph, ascitic or cystic fluid, urine, or nipple exudate.

Methods for preparing total and poly (A)+RNA are well known and are described generally in Sambrook et al., MOLECULAR CLONING—A LABORATORY MANUAL (2ND ED.), Vols. 1-3, Cold Spring Harbor Laboratory, Cold Spring Harbor, N.Y. (1989)) and Ausubel et al., Current Protocols in Molecular Biology vol. 2, Current Protocols Publishing, New York (1994)). RNA may be isolated from ER-positive tumor cells by any procedures well-known in the art, generally involving lysis of the cells and denaturation of the proteins contained therein.

As an example of preparing RNA from tissue samples, RNA may also be isolated from FFPE tissues using techniques well known in the art. Commercial kits for this purpose may be obtained, e.g., from Zymo Research, Ambion, Qiagen, or Stratagene. An exemplary method of isolating total RNA from FFPE tissue, according to the method of the Pinpoint Slide RNA Isolation System (Zymo Research, Orange, Calif.) is as follows. Briefly, the solution obtained from the Zymo kit is applied over the selected FFPE tissue region of interest and allowed to dry. The embedded tissue is then removed from the slide and placed in a centrifuge tube with proteinase K. The tissue is incubated for several hours, then the cell lysate is centrifuged and the supernatant transferred to another tube. RNA is extracted from the lysate by means of a guanidinium thiocynate/β mercaptoethanol solution, to which ethanol is added and mixed. Sample is applied to a spin column, and spun one minute. The column is washed with buffer containing ethanol and Tris/EDTA. DNase is added to the column, and incubated. RNA is eluted from the column by adding heated RNase-free water to the column and centrifuging. Pure total RNA is present in the eluate.

Additional steps may be employed to remove contaminating DNA, such as the addition of DNase to the spin column, described above. Cell lysis may be accomplished with a nonionic detergent, followed by micro-centrifugation to remove the nuclei and hence the bulk of the cellular DNA. In one embodiment, RNA is extracted from cells of the various types of interest by cell lysis in the presence of guanidinium thiocyanate, followed by CsCl centrifugation to separate the RNA from DNA (Chirgwin et al., Biochemistry 18:5294-5299 (1979)). Poly(A)+RNA is selected with oligo-dT cellulose (see Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol.

If desired, RNase inhibitors may be added to the lysis buffer. Likewise, for certain cell types it may be desirable to add a protein denaturation/digestion step to the protocol.

For many applications, it is desirable to preferentially enrich mRNA with respect to other cellular RNAs extracted from cells, such as transfer RNA (tRNA) and ribosomal RNA (rRNA). Most mRNAs contain poly(A) tails at their 3′ ends. This allows for enrichment by affinity chromatography; for example, using oligo(dT) or poly(U) coupled to a solid support, such as cellulose or Sephadex™ (see Ausubel et al., CURRENT PROTOCOLS IN MOLECULAR BIOLOGY, vol. 2, Current Protocols Publishing, New York (1994). After being bound in this manner, poly(A)+ mRNA is eluted from the affinity column using 2 mM EDTA/0.1% SDS.

The sample of RNA can comprise a plurality of different mRNA molecules, each mRNA molecule having a different nucleotide sequence. In exemplary embodiments of the invention, the mRNA molecules of the RNA sample comprise mRNA corresponding to each of the 14 genes disclosed herein (Table 2), or a subset thereof (such as any 5, 6, 7, 8, 9, 10, 11, 12, or 13 of these 14 genes). In certain embodiments, total RNA or mRNA from cells are used in the methods of the invention. The source of the RNA can be cells from any ER-positive tumor cell. In specific embodiments, the methods of the invention are used with a sample containing total mRNA or total RNA from 1×10⁶ cells or fewer.

### Reagents for Measuring Gene Expression

Certain exemplary embodiments of the invention provide nucleic acid molecules that can be used in gene expression profiling and in determining response to breast cancer chemotherapy and/or prognosis of breast cancer metastasis. Exemplary nucleic acid molecules that can be used as primers in gene expression profiling of the 14-gene signature described herein (or any subset thereof) are shown in Table 3.

As indicated in Table 3:

Gene BUB 1 can be reverse-transcribed and amplified with SEQ ID NO: 1 as the Upper primer (5′), and SEQ ID NO: 2 as the Lower primer (3′).

Gene CCNB1 can be reverse-transcribed and amplified with SEQ ID NO: 3 as the Upper primer (5′), and SEQ ID NO: 4 as the Lower primer (3′).

Gene CENPA can be reverse-transcribed and amplified with SEQ ID NO: 5 as the Upper primer (5′), and SEQ ID NO: 6 as the Lower primer (3′).

Gene DC13 can be reverse-transcribed and amplified with SEQ ID NO: 7 as the Upper primer (5′), and SEQ ID NO: 8 as the Lower primer (3′).

Gene DIAPH3 can be reverse-transcribed and amplified with SEQ ID NO: 9 as the Upper primer (5′), and SEQ ID NO: 10 as the Lower primer (3′).

Gene MELK can be reverse-transcribed and amplified with SEQ ID NO: 11 as the Upper primer (5′), and SEQ ID NO: 12 as the Lower primer (3′).

Gene MYBL2 can be reverse-transcribed and amplified with SEQ ID NO: 13 as the Upper primer (5′), and SEQ ID NO: 14 as the Lower primer (3′).

Gene NUP214 can be reverse-transcribed and amplified with SEQ ID NO: 29 as the Upper primer (5′), and SEQ ID NO: 30 as the Lower primer (3′).

Gene ORC6L can be reverse-transcribed and amplified with SEQ ID NO: 15 as the Upper primer (5′), and SEQ ID NO: 16 as the Lower primer (3′).

Gene PKMYT1 can be reverse-transcribed and amplified with SEQ ID NO: 17 as the Upper primer (5′), and SEQ ID NO: 18 as the Lower primer (3′).

Gene PPIG can be reverse-transcribed and amplified with SEQ ID NO: 31 as the Upper primer (5′), and SEQ ID NO: 32 as the Lower primer (3′).

Gene PRR11 can be reverse-transcribed and amplified with SEQ ID NO: 19 as the Upper primer (5′), and SEQ ID NO: 20 as the Lower primer (3′).

Gene RACGAP1 can be reverse-transcribed and amplified with SEQ ID NO: 21 as the Upper primer (5′), and SEQ ID NO: 22 as the Lower primer (3′).

Gene RFC4 can be reverse-transcribed and amplified with SEQ ID NO: 23 as the Upper primer (5′), and SEQ ID NO: 24 as the Lower primer (3′).

Gene SLUT can be reverse-transcribed and amplified with SEQ ID NO: 33 as the Upper primer (5′), and SEQ ID NO: 34 as the Lower primer (3′).

Gene TK1 can be reverse-transcribed and amplified with SEQ ID NO: 25 as the Upper primer (5′), and SEQ ID NO: 26 as the Lower primer (3′).

Gene UBE2S can be reverse-transcribed and amplified with SEQ ID NO: 27 as the Upper primer (5′), and SEQ ID NO: 28 as the Lower primer (3′).

Exemplary probes, as well as alternative exemplary primers, that can be used to detect expression of these genes (optionally in conjunction with the primers disclosed herein in Table 3) are disclosed in U.S. Pat. No. 8,557,525 (Wang et al., “A Composite Metastasis Score with Weighted Coefficients”, filed Feb. 27, 2012, issued Oct. 15, 2013), particularly in Table 37 therein, which is incorporated herein by reference in its entirety (with Table 37 therein being specifically incorporated herein by reference).

Based on the complete nucleotide sequence of each gene, such as provided in the corresponding RefSeq accession number or journal citation for each gene in Table 2, one skilled in the art can readily design and synthesize additional primers and/or probes that can be used in the amplification and/or detection of the 14-gene signature described herein, or any subset thereof.

In certain exemplary embodiments of the invention, the sequences disclosed in Table 3 can be used as gene expression profiling reagents. As used herein, a “gene expression profiling reagent” is a reagent that is specifically useful in the process of amplifying and/or detecting the nucleotide sequence (e.g., an expressed mRNA transcript, or cDNA) of a specific target gene described herein. Typically, such a profiling reagent hybridizes to a target nucleic acid molecule by complementary base-pairing in a sequence-specific manner, and discriminates the target sequence from other nucleic acid sequences in a test sample. An example of a detection reagent is a probe that hybridizes to a target nucleic acid containing a nucleotide sequence substantially complementary to one of the sequences provided in Table 3. In a preferred embodiment, such a probe can differentiate between nucleic acids of different genes. A gene expression profiling reagent can optionally differentiate between different nucleotide sequences of alternative mRNA transcripts of a given gene, thereby allowing the identity and quantification of alternative mRNA transcripts to be determined. Another example of a detection reagent is a primer which acts as an initiation point for nucleotide extension along a complementary strand of a target polynucleotide, as in reverse transcription or PCR. The sequence information provided herein is also useful, for example, for designing primers to reverse transcribe and/or amplify (e.g., using PCR) any of the genes disclosed herein.

In certain exemplary embodiment of the invention, a detection reagent is an isolated or synthetic DNA or RNA polynucleotide probe or primer or PNA oligomer, or a combination of DNA, RNA and/or PNA, that hybridizes to a segment of a target nucleic acid molecule corresponding to any of the genes disclosed in Table 2. A detection reagent in the form of a polynucleotide may optionally contain modified base analogs, intercalators or minor groove binders. Multiple detection reagents such as probes may be, for example, affixed to a solid support (e.g., arrays or beads) or supplied in solution (e.g., probe/primer sets for enzymatic reactions such as PCR, RT-PCR, TaqMan® assays, or primer-extension reactions) to form an expression profiling kit.

A probe or primer typically is a substantially purified oligonucleotide or PNA oligomer. Such an oligonucleotide typically comprises a region of complementary nucleotide sequence that hybridizes under stringent conditions to at least about 8, 10, 12, 16, 18, 20, 22, 25, 30, 40, 50, 55, 60, 65, 70, 80, 90, 100, 120 (or any other number in-between) or more consecutive nucleotides in a target nucleic acid molecule.

Other preferred primer and probe sequences can be determined using the nucleotide sequences of genes provided in the corresponding RefSeq accession numbers or journal citations for each gene in Table 2. Such primers and probes are directly useful as reagents for expression profiling of the genes disclosed herein, and can be incorporated into any kit or system format.

In order to produce a probe or primer specific for a target gene sequence, the gene/transcript sequence can be analyzed using a computer algorithm which starts at the 5′ or at the 3′ end of the nucleotide sequence. Exemplary algorithms can then identify oligomers of defined length that are unique to the gene sequence, have a GC content within a range suitable for hybridization, lack predicted secondary structure that may interfere with hybridization, and/or possess other desired characteristics or that lack other undesired characteristics.

In exemplary embodiments, a primer or probe is typically at least about 8 nucleotides in length. In one embodiment of the invention, a primer or a probe is at least about 10 nucleotides in length. In another embodiment, a primer or a probe is at least about 12 nucleotides in length. In further embodiments, a primer or probe is at least about 14, 15, 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific embodiment of the invention, a primer or a probe is within the length of about 18 and about 28 nucleotides. However, in other embodiments, such as nucleic acid arrays and other embodiments in which probes are affixed to a substrate, the probes can be longer, such as on the order of 30-70, 75, 80, 90, 100, or more nucleotides in length.

The invention encompasses nucleic acid analogs that contain modified, synthetic, or non-naturally occurring nucleotides or structural elements or other alternative/modified nucleic acid chemistries known in the art. Such nucleic acid analogs are useful, for example, as detection reagents (e.g., primers/probes) for detecting one or more of the genes identified in Table 2. Furthermore, kits/systems (such as beads, arrays, etc.) that include these analogs are also encompassed by the invention. For example, PNA oligomers are an exemplary type of DNA analog that are encompassed by the invention, in which the phosphate backbone is replaced with a peptide-like backbone (Lagriffoul et al., Bioorganic & Medicinal Chemistry Letters 4:1081-1082 [1994], Petersen et al., Bioorganic & Medicinal Chemistry Letters 6:793-796 [1996], Kumar et al., Organic Letters 3[9]:1269-1272 [2001], WO96/04000). PNA hybridizes to complementary RNA or DNA with higher affinity and specificity than conventional oligonucleotides and oligonucleotide analogs. The properties of PNA enable novel molecular biology and biochemistry applications unachievable with traditional oligonucleotides and peptides.

Additional examples of nucleic acid modifications that improve the binding properties and/or stability of a nucleic acid include the use of base analogs such as inosine, intercalators (U.S. Pat. No. 4,835,263) such as ethidium bromide and SYBR® Green, and the minor groove binders (U.S. Pat. No. 5,801,115). Thus, references herein to nucleic acid molecules, expression profiling reagents (e.g., probes and primers), and oligonucleotides/polynucleotides include PNA oligomers and other nucleic acid analogs. Other examples of nucleic acid analogs and alternative/modified nucleic acid chemistries known in the art are described in Current Protocols in Nucleic Acid Chemistry, John Wiley & Sons, New York (2002).

While the design of each allele-specific primer or probe may depend on variables such as the precise composition of the nucleotide sequences in a target nucleic acid molecule and the length of the primer or probe, another factor in the use of primers and probes is the stringency of the conditions under which the hybridization between the probe or primer and the target sequence is performed. Higher stringency conditions utilize buffers with lower ionic strength and/or a higher reaction temperature, and tend to require a closer match between the probe/primer and target sequence in order to form a stable duplex. If the stringency is too high, however, hybridization may not occur at all. In contrast, lower stringency conditions utilize buffers with higher ionic strength and/or a lower reaction temperature, and permit the formation of stable duplexes with more mismatched bases between a probe/primer and a target sequence. By way of example but not limitation, exemplary conditions for high-stringency hybridization conditions using an allele-specific probe are as follows: prehybridization with a solution containing 5× standard saline phosphate EDTA (SSPE), 0.5% NaDodSO₄ (SDS) at 55° C., and incubating probe with target nucleic acid molecules in the same solution at the same temperature, followed by washing with a solution containing 2×SSPE, and 0.1% SDS at 55° C. or room temperature.

Moderate-stringency hybridization conditions may be used for primer extension reactions with a solution containing, e.g., about 50 mM KCl at about 46° C. Alternatively, the reaction may be carried out at an elevated temperature such as 60° C. In certain embodiments, a moderately-stringent hybridization condition is suitable for oligonucleotide ligation assay (OLA) reactions, wherein two probes are ligated if they are completely complementary to the target sequence, and may utilize a solution of about 100 mM KCl at a temperature of 46° C.

In a hybridization-based assay, specific probes can be designed that hybridize to a segment of target DNA of one gene sequence but do not hybridize to sequences from other genes. Hybridization conditions should be sufficiently stringent that there is a significant detectable difference in hybridization intensity between genes, and preferably an essentially binary response, whereby a probe hybridizes to only one of the gene sequences or significantly more strongly to one gene sequence. While a probe may be designed to hybridize to a target sequence of a specific gene such that the target site aligns anywhere along the sequence of the probe, the probe is preferably designed to hybridize to a segment of the target sequence such that the gene sequence aligns with a central position of the probe (e.g., a position within the probe that is at least three nucleotides from either end of the probe). This design of probe generally achieves good discrimination in hybridization between different genes.

Oligonucleotide probes and primers may be prepared by methods well known in the art. Chemical synthetic methods include, but are not limited to, the phosphotriester method described by Narang et al., Methods in Enzymology 68:90 [1979]; the phosphodiester method described by Brown et al., Methods in Enzymology 68:109 [1979], the diethylphosphoamidate method described by Beaucage et al., Tetrahedron Letters 22:1859 [1981]; and the solid support method described in U.S. Pat. No. 4,458,066. In the case of an array, multiple probes can be immobilized on the same support for simultaneous analysis of multiple different gene sequences.

In one exemplary type of PCR-based assay, a gene-specific primer hybridizes to a region on a target nucleic acid molecule that overlaps a gene sequence and only primes amplification of the gene sequence to which the primer exhibits perfect complementarity (Gibbs, Nucleic Acid Res. 17:2427-2448 [1989]). Typically, the primer's 3′-most nucleotide is aligned with and complementary to the target nucleic acid molecule. This primer is used in conjunction with a second primer that hybridizes at a distal site. Amplification proceeds from the two primers, producing a detectable product that indicates which gene/transcript is present in the test sample. This PCR-based assay can be utilized as part of a TaqMan® assay.

The genes (or their expression products, such as mRNA transcripts) of the 14-gene signature described herein, or any subset thereof, can be detected by any of a variety of nucleic acid amplification methods, which are used to increase the copy numbers of a polynucleotide of interest in a nucleic acid sample. Examples of such amplification methods include, but are not limited to, polymerase chain reaction (PCR) (U.S. Pat. Nos. 4,683,195 and 4,683,202; PCR Technology: Principles and Applications for DNA Amplification, ed. H. A. Erlich, Freeman Press, New York, N.Y. [1992]), ligase chain reaction (LCR) (Wu and Wallace, Genomics 4:560 [1989]; Landegren et al., Science 241:1077 [1988]), strand displacement amplification (SDA) (U.S. Pat. Nos. 5,270,184 and 5,422,252), transcription-mediated amplification (TMA) (U.S. Pat. No. 5,399,491), linked linear amplification (LLA) (U.S. Pat. No. 6,027,923), and the like, and isothermal amplification methods such as nucleic acid sequence based amplification (NASBA), and self-sustained sequence replication (Guatelli et al., Proc. Natl. Acad. Sci. USA 87: 1874 [1990]). Based on such methodologies, a person skilled in the art can readily design primers in any suitable regions 5′ and 3′ of the gene sequences of interest, so as to amplify the genes disclosed herein. Such primers may be used to reverse-transcribe and amplify DNA of any length, such that it contains the gene of interest.

Generally, an amplified polynucleotide is at least about 16 nucleotides in length. More typically, an amplified polynucleotide is at least about 20 nucleotides in length. In a certain embodiment of the invention, an amplified polynucleotide is at least about 30 nucleotides in length. In further embodiments of the invention, an amplified polynucleotide is at least about 32, 36, 40, 45, 50, or 60 nucleotides in length. In yet further embodiments of the invention, an amplified polynucleotide is at least about 100, 200, 300, 400, or 500 nucleotides in length. While the total length of an amplified polynucleotide of the invention can be as long as an exon, an intron or the entire gene, an amplified product is typically up to about 1,000 nucleotides in length (although certain amplification methods may generate amplified products greater than 1,000 nucleotides in length). In certain embodiments, an amplified polynucleotide is not greater than about 150, 200, 250, 300, 400, 500, or 600 nucleotides in length.

In an embodiment of the invention, a gene expression profiling reagent of the invention is labeled with a fluorogenic reporter dye that emits a detectable signal. While the preferred reporter dye is a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red.

In certain embodiments of the invention, the detection reagent may be further labeled with a quencher dye such as Tamra, for example when the reagent is used as a self-quenching probe such as a TaqMan® (U.S. Pat. Nos. 5,210,015 and 5,538,848) or Molecular Beacon probe (U.S. Pat. Nos. 5,118,801 and 5,312,728), or other stemless or linear beacon probe (Livak et al., PCR Method Appl. 4:357-362 [1995]; Tyagi et al., Nature Biotechnology 14:303-308 [1996]; Nazarenko et al., Nucl. Acids Res. 25:2516-2521 [1997]; U.S. Pat. Nos. 5,866,336 and 6,117,635).

Exemplary detection reagents of the invention may also contain other labels, including but not limited to, biotin for streptavidin binding, hapten for antibody binding, and an oligonucleotide for binding to another complementary oligonucleotide such as pairs of zipcodes.

### Gene Expression Kits and Systems

A person skilled in the art will recognize that, based on the gene and sequence information disclosed herein, expression profiling reagents can be developed and used to assay any genes disclosed herein individually or in combination, and such detection reagents can be incorporated into one of the established kit or system formats which are well known in the art. The terms “kits” and “systems,” as used herein in the context of gene expression profiling reagents, are intended to refer to such things as combinations of multiple gene expression profiling reagents, or one or more gene expression profiling reagents in combination with one or more other types of elements or components (e.g., other types of biochemical reagents, containers, packages such as packaging intended for commercial sale, substrates to which gene expression profiling reagents are attached, electronic hardware components, etc.). Accordingly, exemplary embodiments of the invention further provide gene expression profiling kits and systems, including but not limited to, packaged probe and primer sets (e.g., TaqMan® probe/primer sets), arrays/microarrays of nucleic acid molecules, and beads that contain one or more probes, primers, or other detection reagents for profiling one or more genes disclosed herein. The kits/systems can optionally include various electronic hardware components; for example, arrays (“DNA chips”) and microfluidic systems (“lab-on-a-chip” systems) provided by various manufacturers may comprise hardware components. Other kits/systems (e.g., probe/primer sets) may not include electronic hardware components, but may be comprised of, for example, one or more gene expression profiling reagents (along with, optionally, other biochemical reagents) packaged in one or more containers.

In some embodiments, a gene expression profiling kit typically contains one or more detection reagents and other components (e.g., a buffer, enzymes such as reverse transcriptase, DNA polymerases or ligases, reverse transcription and chain extension nucleotides such as deoxynucleotide triphosphates, and in the case of Sanger-type DNA sequencing reactions, chain terminating nucleotides such as ddNTPs, positive control nucleic acid, negative controls, and the like) necessary to carry out an assay or reaction, such as reverse transcription, amplification and/or detection of a gene-containing nucleic acid molecule. A kit may further contain means for determining the amount of a target nucleic acid, and means for comparing the amount with a standard, and can comprise instructions for using the kit to detect the target nucleic acid molecule of interest. In certain embodiments of the invention, kits are provided which contain the necessary reagents to carry out one or more assays to profile the expression of one or more of the genes disclosed herein. In certain embodiments of the invention, gene expression profiling kits/systems are in the form of nucleic acid arrays, or compartmentalized kits, including microfluidic/lab-on-a-chip systems.

Gene expression profiling kits/systems may contain, for example, one or more probes, or pairs of probes, that hybridize to a nucleic acid molecule at or near each target gene sequence position. Multiple pairs of gene-specific probes may be included in the kit/system to simultaneously assay large numbers of genes, at least one of which is a gene disclosed herein. In some kits/systems, the gene-specific probes are immobilized to a substrate such as an array or bead. For example, the same substrate can comprise gene-specific probes for detecting at least one or all of the genes shown in Table 2, or any subset thereof.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In one embodiment, the microarray is prepared and used according to the methods described in U.S. Pat. No. 5,837,832 (Chee et al.), PCT application WO95/11995 (Chee et al.), Lockhart, D. J. et al. (Nat. Biotech. 14:1675-1680 [1996]) and Schena, M. et al. (Proc. Natl. Acad. Sci. 93:10614-10619 [1996]), all of which are incorporated herein in their entirety by reference. In other embodiments, such arrays are produced by the methods described by Brown et al., U.S. Pat. No. 5,807,522.

Nucleic acid arrays are reviewed in the following references: Zammatteo et al., “New chips for molecular biology and diagnostics,” Biotechnol. Annu. Rev. 8:85-101 (2002); Sosnowski et al., “Active microelectronic array system for DNA hybridization, genotyping and pharmacogenomic applications,” Psychiatr. Genet. 12(4):181-92 (December 2002); Heller, “DNA microarray technology: devices, systems, and applications,” Annu. Rev. Biomed. Eng. 4:129-53 (2002); Epub Mar. 22 2002; Kolchinsky et al., “Analysis of SNPs and other genomic variations using gel-based chips,” Hum. Mutat. 19(4):343-60 (April 2002); and McGall et al., “High-density genechip oligonucleotide probe arrays,” Adv. Biochem. Eng. Biotechnol. 77:21-42 (2002).

Any number of probes, such as gene-specific probes, may be implemented in an array, and each probe or pair of probes can hybridize to a different gene sequence position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides, usually either synthetic antisense polynucleotides or fragments of cDNAs, fixed to a solid support. Typical polynucleotides are preferably about 6-60 nucleotides in length, more preferably about 15-30 nucleotides in length, and most preferably about 18-25 nucleotides in length. For certain types of microarrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length. The microarray or detection kit can contain polynucleotides that cover the known 5′ or 3′ sequence of a gene/transcript, sequential polynucleotides that cover the full-length sequence of a gene/transcript; or unique polynucleotides selected from particular areas along the length of a target gene/transcript sequence, particularly areas corresponding to one or more genes disclosed in Table 2. Polynucleotides used in the microarray or detection kit can be specific to a gene(s)/transcript(s) of interest (e.g., specific to a particular signature sequence within a target gene sequence), or specific to a variant form of a gene(s)/transcript(s) of interest.

Hybridization assays based on polynucleotide arrays typically rely on the differences in hybridization stability of the probes to perfectly matched and mismatched target sequences.

In other embodiments, the arrays are used in conjunction with chemiluminescent detection technology. The following patents and patent applications, which are all herein incorporated by reference in their entirety, provide additional information pertaining to chemiluminescent detection: U.S. patent application Ser. Nos. 10/620,332 and 10/620,333 describe chemiluminescent approaches for microarray detection; U.S. Pat. Nos. 6,124,478, 6,107,024, 5,994,073, 5,981,768, 5,871,938, 5,843,681, 5,800,999, and 5,773,628 describe methods and compositions of dioxetane for performing chemiluminescent detection; and U.S. published application US2002/0110828 discloses methods and compositions for microarray controls.

In certain embodiments of the invention, a nucleic acid array can comprise an array of probes of about 15-25 nucleotides in length. In further embodiments, a nucleic acid array can comprise any number of probes, in which at least one probe is capable of detecting one or more genes disclosed in Table 2, and/or at least one probe comprises a fragment of one of the gene sequences selected from the group consisting of those disclosed in Table 2, and sequences complementary thereto, said fragment comprising at least about 8, 10, 12, 15, 16, 18, 20, 22, 25, 30, 40, 47, 50, 55, 60, 65, 70, 80, 90, 100, or more consecutive nucleotides (or any other number in-between) and containing (or being complementary to) a sequence of a gene disclosed in Table 2.

A polynucleotide probe can be synthesized on the surface of the substrate by using a chemical coupling procedure and an ink jet application apparatus, as described in PCT application WO95/251116 (Baldeschweiler et al.) which is incorporated herein in its entirety by reference. In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536, 6144 or more polynucleotides, or any other number which lends itself to the efficient use of commercially available instrumentation.

Using such arrays or other kits/systems, certain exemplary embodiments of the invention provide methods of identifying and profiling expression of the genes disclosed herein in a test sample. Such methods typically involve incubating a test sample of nucleic acids with an array comprising one or more probes corresponding to at least one gene disclosed herein, and assaying for binding of a nucleic acid from the test sample with one or more of the probes. Conditions for incubating a gene expression profiling reagent (or a kit/system that employs one or more such gene expression profiling reagents) with a test sample vary. Incubation conditions depend on factors such as the format employed in the assay, the profiling methods employed, and the type and nature of the profiling reagents used in the assay. One skilled in the art will recognize that any one of the commonly available hybridization, amplification and array assay formats can readily be adapted to detect expression of the genes disclosed herein.

An exemplary gene expression profiling kit/system of the invention may include components that are used to prepare nucleic acids from a test sample for the subsequent reverse transcription, RNA enrichment, amplification and/or detection of a gene sequence-containing nucleic acid molecule. Such sample preparation components can be used to produce nucleic acid extracts (including DNA, cDNA and/or RNA) from any tumor tissue source, including but not limited to, fresh tumor biopsy, frozen or FFPE tissue specimens, or tumors collected and preserved by any method. The test samples used in the above-described methods will vary based on such factors as the assay format, nature of the profiling method, and the specific tissues, cells or extracts used as the test sample to be assayed. Methods of preparing nucleic acids are well known in the art and can be readily adapted to obtain a sample that is compatible with the system utilized. Automated sample preparation systems for extracting nucleic acids from a test sample are commercially available, and examples include Qiagen's BioRobot 9600, Applied Biosystems' PRISM 6700, and Roche Molecular Systems' COBAS AmpliPrep System.

Another exemplary type of kit is a compartmentalized kit. A compartmentalized kit includes any kit in which reagents are contained in separate containers. Such containers include, for example, small glass containers, plastic containers, strips of plastic, glass or paper, or arraying material such as silica. Such containers allow one to efficiently transfer reagents from one compartment to another compartment such that the test samples and reagents are not cross-contaminated, or from one container to another vessel not included in the kit, and the agents or solutions of each container can be added in a quantitative fashion from one compartment to another or to another vessel. Such containers may include, for example, one or more containers which will accept the test sample, one or more containers which contain at least one probe or other gene expression profiling reagent for profiling the expression of one or more genes disclosed herein, one or more containers which contain wash reagents (such as phosphate buffered saline, Tris-buffers, etc.), and one or more containers which contain reagents used to reveal the presence of the bound probe or other gene expression profiling reagents. The kit can optionally further comprise compartments and/or reagents for, for example, reverse transcription, RNA enrichment, nucleic acid amplification or other enzymatic reactions such as primer extension reactions, hybridization, ligation, electrophoresis (preferably capillary electrophoresis), mass spectrometry, and/or laser-induced fluorescent detection. The kit may also include instructions for using the kit. Exemplary compartmentalized kits include microfluidic devices known in the art (see, e.g., Weigl et al., “Lab-on-a-chip for drug development,” Adv. Drug Deliv. Rev. 24, 55[3]:349-77 [February 2003]). In such microfluidic devices, the containers may be referred to as, for example, microfluidic “compartments,” “chambers,” or “channels.”

### Uses of Gene Expression Profiling Reagents

The exemplary nucleic acid molecules provided in Table 3 have a variety of uses, especially in uses related to determining response to breast cancer chemotherapy and/or risk of breast cancer metastasis. For example, the nucleic acid molecules are useful as amplification primers or hybridization probes, such as for detecting expression (e.g., mRNA) of any of the genes provided in Table 2, particularly all 14 genes of the metastasis score or any subset thereof, such as the exemplary 12-gene signature disclosed herein, or these 12 or 14 genes plus additional genes.

A probe can hybridize to any nucleotide sequence along the entire length of a target nucleic acid molecule. For example, a probe may hybridize to a region of any of the genes indicated in Table 2. Typically, a probe hybridizes to a target nucleic acid molecule in a sequence-specific manner such that the probe distinguishes the target nucleic acid molecule from other nucleic acid molecules which may be present in a test sample (e.g., mRNA molecules expressed by other genes).

Thus, the exemplary nucleic acid molecules provided herein, such as those provided in Table 3, can be used as hybridization probes, reverse transcription and/or amplification primers to detect the expression levels of the genes disclosed herein, such as to determine whether an individual with breast cancer is likely to respond to chemotherapy and/or is at risk for distant metastasis. Expression profiling of the genes disclosed herein provides a tool for predicting a breast cancer patient's response to chemotherapy and/or prognosing their risk for distant metastasis.

### Generation of the Metastasis Score

Expression levels of the fourteen genes disclosed in Table 2, or a subset thereof (particularly a subset comprising or consisting of all 12 of the genes CENPA, PKMYT1, MELK, BUB1, RACGAP1, TK1, UBE2S, DC13, RFC4, PRR11, DIAPH3, and ORC6L), can optionally be used to derive a metastasis score (MS) predictive of chemotherapy response and/or metastasis risk in breast cancer. Expression levels may be calculated by the Δ(ΔCₜ) method, where Ct=the threshold cycle for target amplification; e.g., the cycle number during PCR at which exponential amplification of the target nucleic acid begins. (KJ Livak and TD Schmittgen, 2001, Methods 25:402-408). The level of mRNA of each of the 14 profiled genes (or a subset thereof) may be defined as:

Δ(ΔCt)=(Ct_{GOI}−Ct_{EC})_{test RNA}−(Ct_{GOI}−Ct_{EC})_{ref RNA}

where GOI=gene of interest (each of the 14 signature genes, or a subset thereof such as the 12 genes CENPA, PKMYT1, MELK, BUB1, RACGAP1, TK1, UBE2S, DC13, RFC4, PRR11, DIAPH3, and ORC6L), test RNA=RNA obtained from the patient sample, ref RNA=a calibrator reference RNA, and EC=an endogenous control. The expression level of each signature gene may optionally be first normalized to one or more control genes, such as the three endogenous control genes listed in Table 2 (EC). A Ct representing the average of the Cts obtained from amplification of the three endogenous controls (Ct_{EC}) can be used to minimize the risk of normalization bias that may occur if only one control gene were used (T. Suzuki, P J Higgins et al., 2000, Biotechniques 29:332-337). Exemplary primers that can be used to amplify the endogenous control genes are listed in Table 3. The adjusted expression level of the gene of interest may optionally be further normalized to a calibrator reference RNA pool, such as ref RNA (universal human reference RNA, Stratagene, La Jolla, Calif.). This can be used to standardize expression results obtained from various machines.

In exemplary embodiments, the gene expression level (which can be represented by a Δ(ΔCt) value, such as calculated above) for each profiled gene is added together to obtain a score (referred to herein as a metastasis score (MS)) that represents the sum of the gene expression levels (e.g., the sum of the Δ(ΔCt) values) for all of the profiled genes. In exemplary embodiments, the gene expression levels (e.g., Δ(ΔCt) values) for each of the genes is equally weighted when they are added together to obtain the score, however in alternative embodiments, the gene expression levels (e.g., Δ(ΔCt) values) for each of the genes can be differentially weighted (e.g., each gene can optionally be weighted by a particular constant value, such as the exemplary ai value provided for each gene in Table 2). Thus, in certain exemplary embodiments, the score (referred to herein as a MS) is the sum of the expression levels, as indicated by Δ(ΔCt) values, for all of the profiled genes (e.g., all 14 genes; or all 12 of the genes CENPA, PKMYT1, MELK, BUB1, RACGAP1, TK1, UBE2S, DC13, RFC4, PRR11, DIAPH3, and ORC6L; or any other subcombination of the 14 genes disclosed herein).

The Δ(ΔCt) value, obtained in gene expression profiling for each of the 14 signature genes, may also be used in the following formula to generate a metastasis score (MS):

$$M S = a 0 + {\sum }_{i = 1}^{M} ai * Gi$$

in which Gi represents the expression level (e.g., a Δ(ΔCt) value) of each gene (i) of the 14-gene signature. The value of Gi is the Δ(ΔCt) obtained in expression profiling described above. An exemplary constant ai for each gene i is provided in Table 2, which can be utilized in embodiments in which each gene is differentially weighted; however in other embodiments, each gene can be equally weighted (in which instance ai=1 for each gene). In certain embodiments, the constant a0=0.022 (as an example value, however other values can be used instead); this value (which is optional) centers the MS so that its median value is zero. M is the number of genes in the component list, such as 14 in exemplary embodiments. However, this can also be a subset of 14, such as any 5, 6, 7, 8, 9, 10, 11, 12, or 13 of the genes (for example, M=12 in embodiments in which gene expression levels are detected for the 12 genes CENPA, PKMYT1, MELK, BUB1, RACGAP1, TK1, UBE2S, DC13, RFC4, PRR11, DIAPH3, and ORC6L), or can be greater than 14 for combinations comprising the 14 genes plus additional genes. Thus, in exemplary embodiments, the MS is a measure of the summation of expression levels for the 14 genes disclosed in Table 2 (or a subset thereof, such as 5, 6, 7, 8, 9, 10, 11, 12, or 13 of the genes, or the 14 genes plus additional genes), each optionally multiplied by a particular constant ai (exemplary values of which are also provided in Table 2 for each gene) or equally weighted (e.g., ai=1 for each gene), and this summation is optionally added to a centering constant (e.g., 0.022) to derive the MS.

Alternatively, the Δ(ΔCt) value, obtained in gene expression profiling for each of the 14 signature genes (or a subset thereof, such as 5, 6, 7, 8, 9, 10, 11, 12, or 13 of the genes; particularly the 12 genes CENPA, PKMYT1, MELK, BUB1, RACGAP1, TK1, UBE2S, DC13, RFC4, PRR11, DIAPH3, and ORC6L; or the 12 or 14 genes plus additional genes), may be used in the following formula to generate a metastasis score (MS):

$$M S = a 0 + b * {\sum }_{i = 1}^{M} ai * Gi$$

in which Gi represents the standardized expression level (e.g., a Δ(ΔCt) value) of each gene (i) of the 14-gene signature (or subset thereof, such as the 12 genes CENPA, PKMYT1, MELK, BUB1, RACGAP1, TK1, UBE2S, DC13, RFC4, PRR11, DIAPH3, and ORC6L). The value of Gi can be obtained by subtracting the mean gene expression from the original expression level measured in Δ(ΔCt) obtained in expression profiling described above and then divided by the standard deviation of the gene expression in the training set. An exemplary constant ai for each gene i is provided in Table 2, which can be utilized in embodiments in which each gene is differentially weighted; however in other embodiments, each gene can be equally weighted (in which instance ai=1 for each gene). In certain embodiments, the constant b is −0.251 (which is an example value that was derived from a univariate Cox model with the principal component as a predictor, to get the correct sign and scaling; however, other alternative values can be used for b). In certain embodiments, the constant a0=0.022 (as an example value); this centers the MS so that its median value is zero (however, use of the centering constant is optional, and other alternative values can be used for the centering constant a0). M is the number of genes in the component list; such as 14 in exemplary embodiments. However, this can also be a subset of 14, such as 5, 6, 7, 8, 9, 10, 11, 12, or 13 (or can be greater than 14 for combinations comprising the 14 genes plus additional genes). For example, M=12 in embodiments in which gene expression levels are detected for the 12 genes CENPA, PKMYT1, MELK, BUB1, RACGAP1, TK1, UBE2S, DC13, RFC4, PRR11, DIAPH3, and ORC6L. Thus, in exemplary embodiments, the MS is a measure of the summation of expression levels for the 14 genes (or a subset thereof) disclosed in Table 2, each optionally multiplied by a particular constant ai (exemplary values for which are also provided in Table 2) or equally weighted (e.g., ai=1 for all genes). This summation can optionally be multiplied by a constant b, and a centering constant (e.g., 0.022) can optionally be added to derive the MS.

As another example, an MS can be calculated as follows:

$$M S ( new ) = - 1 / 14 * [ {\sum }_{i = 1}^{14} Gi ]$$

in which Gi represents the expression level (e.g., as indicated by a Δ(ΔCt) value) of each gene (i) of the 14-gene signature (if less than all 14 genes are utilized, then “14” in the equation above is changed to the number of genes that are utilized; for example, “14” would be replaced by “12” in the equation above if expression of 12 genes was measured, such that the sum of the expression levels of each of the 12 genes would be multiplied by − 1/12).

A sample from a breast cancer patient can be evaluated by generating this metastasis score from the 14-gene expression profiling data for that patient (or a subset of the 14 genes, such as any 5, 6, 7, 8, 9, 10, 11, 12, or 13 of the genes, particularly the 12 genes CENPA, PKMYT1, MELK, BUB1, RACGAP1, TK1, UBE2S, DC13, RFC4, PRR11, DIAPH3, and ORC6L; or any of the gene combinations plus additional genes), and from this metastasis score the likelihood of responding to (benefiting from) chemotherapy and/or the probability of distant metastasis for the patient can be determined.

In certain exemplary embodiments, the MS can be a sum of the Δ(ΔCt) values as described above, in which instance the formula of the MS is simplified by substituting the value of a0 with zero, and the constant ai is one.

Thus, in certain embodiments, the expression of each of the genes used in computing a MS are all equally weighted, such as when using the MS to determine response to chemotherapy and/or to determine risk for tumor metastasis. For example, in certain embodiments, gene expression of all 12 of the genes CENPA, PKMYT1, MELK, BUB1, RACGAP1, TK1, UBE2S, DC13, RFC4, PRR11, DIAPH3, and ORC6L (and optionally additional genes) is detected and weighted equally to determine response to chemotherapy and/or risk for tumor metastasis.

In certain exemplary embodiments, the MS can also be a sum of the values of Δ(ΔCt) as described above, then multiplied by an exemplary constant (e.g., −0.04778) for correct sign and scaling such that distant metastasis risk increases as the MS increases. Finally, a constant (e.g., 0.8657) can be added so that the mean of MS is zero. An MS derived in this alternative way will have equal weighting of all 14 genes (or a subset thereof, or the 14 genes plus additional genes). The risk of distant metastasis would increase as the MS increases (and the likelihood of responding to (benefitting from) chemotherapy would decrease as the MS increases). The two different exemplary MS described here have a very high correlation with the Pearson correlation coefficient (greater than 0.999).

Generation of Distant Metastasis Probability from MS

The probability of distant metastasis for any individual patient can be calculated from the MS at variable time points, using the Weibull distribution as the baseline survival function.

The exemplary metastasis score (MS) obtained above, from expression profiling of the 14-gene signature (or a subset of the 14 genes, such as any 5, 6, 7, 8, 9, 10, 11, 12, or 13 of the genes, in particular the exemplary 12-gene signature disclosed herein, or these genes plus additional genes), can be converted into the probability of distant metastasis by means of the Cox proportional hazard model. Because the Cox model does not specify the baseline hazard function, the hazard and survivor functions can first be constructed through parametric regression models. In the parametric regression models, distant metastasis-free survival time can be the outcome, and the metastasis score (MS) can be the independent variable input. The event time can be assumed to have a Weibull distribution; its two parameters can be estimated using the survival data from which the MS was derived. To calculate the probability of distant metastasis within a certain time for a patient, the MS value can be substituted into the formula for the survivor function.

### Clinical Application of the Metastasis Score in Determining Metastasis Risk or Chemotherapy Response

In certain exemplary embodiments of the invention, such as in methods of using the metastasis score (MS) to determine risk of metastasis and/or to predict response to breast cancer chemotherapy, one or more MS thresholds (also known as a “cut-point”, “cutoff”, “threshold”, or “threshold score”) can be generated. Such MS thresholds can be used as a benchmark when compared to the MS of a breast cancer patient so as to determine whether such patient has either an increased or decreased risk of metastasis and/or an increased or decreased likelihood of responding to chemotherapy. An MS threshold can be determined by various methods and can be different for different definitions of MS, such as whether all 14 genes are utilized or a subset thereof (e.g., the 12 genes CENPA, PKMYT1, MELK, BUB1, RACGAP1, TK1, UBE2S, DC13, RFC4, PRR11, DIAPH3, and ORC6L). Furthermore, the MS threshold may optionally differ depending on whether the MS is used for determining metastasis risk or response to chemotherapy (or both). Furthermore, in alternative embodiments, a continuous analysis can be used rather than a discrete threshold for analyzing or characterizing samples on a continuous scale, such as to determine and/or report an individual's predicted response to chemotherapy and/or risk for metastasis based on their MS.

For example, for an MS as defined in Equation 1 (which is used in Examples 1, 2 and 3 below) and used for determining metastasis risk, an MS threshold can be derived from hazard ratios of high-risk vs. low-risk groups. Kaplan-Meier (KM) curves for distant metastasis-free survival (DMFS) can be generated for the high- and low-risk patient groups defined by MS cut-points. The choice of median MS as cut-point can be based upon the calculation of the hazard ratios of the high vs. low-risk groups using different cut-points from, for example, ten percentile of MS to ninety percentile of MS. As an example, the median cut-point can be defined as the point where there are an equal number of individuals in the high and low-risk groups (in Example 1, this produced near the highest hazard ratio in the training samples). Hazard ratios (HR) and 95% confidence intervals (CI) using the cut-point of median MS can be calculated and reported. Log rank tests can be performed, and the hazard ratios can be calculated for different cut-points. The accuracy and value of the 14-gene signature (or a subset thereof) in predicting distant metastasis at various time points (e.g., five or seven years) can be assessed by various means (XH Zhou, N. Obuchowski et al., eds., 2002, Statistical Methods in Diagnostic Medicine, Wiley-Interscience, New York).

For an MS (such as defined in Equation 2 which is used in Examples 4 and 5 below, or an MS that is the sum of the equally-weighted Δ(ΔCt) values for all of said genes) and used for determining metastasis risk, an MS threshold can be determined from the sensitivity and specificity of the MS in predicting distant metastasis in 5 years (such as in samples from Guy's Hospital as described in Example 2 below). As an example, two MS cut-points can be chosen such that the sensitivity of MS to predict distant metastasis in 5 years is over 90% if the first cut-point is used. The second cut-point can be chosen such that the sensitivity and specificity of the MS to predict distant metastasis in 5 years are both 70%. For an MS as defined in Equation 2, an exemplary first MS threshold is −0.1186 and an exemplary second MS threshold is 0.3019. With two MS cut-points, there are high, intermediate, and low MS groups. As another example, particularly for embodiments in which the 12 genes CENPA, PKMYT1, MELK, BUB1, RACGAP1, TK1, UBE2S, DC13, RFC4, PRR11, DIAPH3, and ORC6L are utilized, an exemplary MS threshold (cut-point) of 15 can be utilized. In certain exemplary embodiments (e.g., in examples described herein for treated samples from Guy's Hospital and treated samples from Aichi Cancer Center in Japan), the high MS group can be designated as a high-risk group, and the intermediate and low MS groups can be designated as a low-risk group. With respect to predicting response to (benefit from) chemotherapy, individuals with a high MS can be designated as less likely to respond to (benefit from) chemotherapy, whereas individuals with a low MS can be designated as more likely to respond to (benefit from) chemotherapy.

### EXAMPLES

The following examples are offered to illustrate, but not to limit, the claimed invention.

### Example One

### The mRNA Expression Levels of a 14-Gene Prognostic Signature Predict Risk for Distant Metastasis in 142 Lymph Node-Negative, ER-Positive Breast Cancer Patients

The following example illustrates how a 14-gene prognostic signature was identified and how it can be used in determining prognosis for distant metastasis in breast cancer patients, even in routine clinical laboratory testing. A clinician can perform mRNA expression profiling on the 14 genes described herein, using RNA obtained from a number of means such as biopsy, FFPE, frozen tissues, etc., and then insert the expression data into an algorithm provided herein to determine a prognostic metastasis score.

FFPE tissue sections obtained from node-negative, ER-positive breast cancer patients were used in the example described below. An initial set of 200 genes were analyzed to derive the final 14-gene signature. Included as candidate genes for this signature were genes previously reported in the literature. Also in this example, the extent of overlap of this signature with routinely used prognostic factors and tools was determined.

Tumors from node-negative, ER− positive patients were selected for this study because prognostic information for node-negative patients would be of great value in guiding treatment strategies. Also, microarray studies indicate that this tumor subset is clinically distinct from other types of breast cancer tumors. (T. Sorlie, C M Perou et al., 2001, Proc Natl Acad Sci USA 98:10869-10874; C. Sotiriou, S Y Neo et al., 2003, Proc Natl Acad Sci USA 100:10393-10398). Genes were chosen for expression profiling from the gene signatures reported by H. Dai (H. Dai, L J van't Veer et al., 2005, Cancer Res 15:4059-4066), L J van't Veer (L J van't Veer, H. Dai et al., 2002, Nature 415:530-536), and SP Paik (SP Paik, S. Shak et al., 2004, N Engl J Med 351:2817-2826), in FFPE sections to determine the robustness of these genes and the extent to which routinely collected and stored clinical samples could be used for prognosis of metastasis. From the gene expression data a metastasis score was developed to estimate distant metastasis probability in individual patients for any timeframe.

### Patients and Samples

A total of 142 node-negative, ER-positive patients with early stage breast cancer were selected, all from patients untreated with systemic adjuvant therapy (Training samples in Table 1). By limiting the study to a subset of breast cancer cases, a molecular signature was identified with a more compelling association with metastasis, more robust across different sample sets, and comprising a smaller number of genes so as to better facilitate translation to routine clinical practice. The mean age of the patients was approximately 62 years (ranging from 31-89 years).

A highly-characterized breast tumor sample set served as the source of samples for this study; the set accrued from 1975 to1986 at the California Pacific Medical Center (CPMC). The inclusion criteria for the primary study included samples from tumors from patients who were lymph-node negative, had received no systemic therapy, and received follow-up care for eight years. Samples were approved for use in this study by the respective institutional medical ethics committees. Patients providing samples were classified as ER-positive based on a measurement of the expression level of the ESR1 gene. Expression level of the ESR1 gene correlates well with an individual's ER status. (M. Cronin, M. Pho et al. 2004, Am J Pathol 164(1):35-42; J M Knowlden, J M Gee et al., 1997, Clin Cancer Res 3:2165-2172).

Distant metastasis-free survival was chosen as the primary endpoint because it is most directly linked to cancer-related death. A secondary endpoint was overall survival.

### Sample Processing

Four 10 μm sections from each paraffin block were used for RNA extraction. The tumor regions were removed based on a guide slide where the cancer cell areas have been marked by a pathologist, and the RNA extracted using Pinpoint Slide RNA Isolation System II (Zymo Research, Orange Calif.).

The yields of total RNA varied between samples. In order to increase the amount of RNA available for analysis, a T7 RNA polymerase linear amplification method was performed on the extracted RNA. RNA isolated from FFPE samples was subjected to T7-based RNA amplification using the MessageAmpII aRNA amplification kit (Ambion, Austin, Tex.).

To assess the consistency of gene expression before and after RNA amplification, a number of experiments were conducted on various genes in different samples. Amplification was first performed on RNA from 67 FFPE samples that were not a part of this study, using 0.1-100 ng of total RNA. Profiling of 20 genes was performed using the resultant enriched RNA and the original, unenriched RNA. These comparisons revealed that the fold enrichment varied from gene to gene; however, the relative expression level was consistent before and after RNA amplification in all 20 genes for 67 samples.

RNA for this study was enriched by amplification with the MessageAmpII aRNA amplification kit, as described above. Total RNA was quantified using spectrophotometric measurements (OD₂₆₀).

### Gene Expression Profiling

Based on a survey of the published literature and results of microarray-based gene expression profiling experiments, 200 candidate genes were initially selected for analysis in order to determine the optimal prognostic signature. This set included genes from the 70-gene prognosis panel described by van't Veer et al. (L J van't Veer, H. Dai et al., 2002, Nature 415:530-536), 104 genes analyzed by Dai et al. (H. Dai, L J van't Veer et al., 2005, Cancer Res 15:4059-4066), the 16-gene panel comprising the signature for response to Tamoxifen treatment reported by Paik et al. (SP Paik, S. Shak et al., 2004, N Engl J Med 351:2817-2826), and 24 ER-related genes as reported by West et al. (M. West, C. Blanchette et al., 2001, Proc Natl Acad Sci USA 98:11462-11467).

Additional genes were selected as endogenous controls (EC, or “control genes”) for normalizing expression data, according to the method described in J. Vandesompele, K. De Preter et al., Genome Biol 3(7): Research 0034.1-0034.11 (Epub 2002). Examples of endogenous controls include “housekeeping genes” such as PPIG, SLUT and NUP214. Six endogenous control genes were tested for the stability of their expression levels in 150 samples of frozen breast cancer tumors. Expression data were analyzed using the geNorm program of Vandesompele et al., in which an M value was determined as a measurement of the stability of a gene's expression level. (J. Vandesompele, K. De Preter et al., Genome Biol 3(7): Research 0034.1-0034.11, Epub 2002). The lower the M value, the more stable the gene. Results are shown in Table 7. The M values indicated that PPIG, SLUT and NUP214 were the most stable endogenous control genes in this sample set, with the least variation in gene expression across samples tested. The stability of these three genes was validated on 138 breast cancer tumor FFPE samples. The results are shown in Table 8.

The expression levels of the selected 200 genes, together with the three EC genes, were profiled in 142 RNA samples. For gene expression profiling, relative quantification by means of one-step reverse-transcription polymerase chain reaction (RT-PCR) was performed. Quantification was “relative” in that the expression of the target gene was evaluated relative to the expression of a set of reference, stably expressed control genes. SYBR® Green intercalating dye (Stratagene, La Jolla, Calif.) was used to visualize amplification product during real-time PCR. Briefly, the reaction mix allowed for reverse transcription of extracted sample RNA into cDNA. This cDNA was then PCR amplified in the same reaction tube, according to the cycling parameters described below. PCR conditions were designed so as to allow the primers disclosed in Table 3, upper and lower, to hybridize 5′ and 3′, respectively, of target sequences of the genes of interest, followed by extension from these primers to create amplification product in repetitive cycles of hybridization and extension. PCR was conducted in the presence of SYBR® Green, a dye which intercalates into double-stranded DNA, to allow for visualization of amplification product. RT-PCR was conducted on the Applied Biosystems Prism® 7900HT Sequence Detection System (Applied Biosystems, Foster City, Calif.), which detected the amount of amplification product present at periodic cycles throughout PCR, using amount of intercalated SYBR® Green as an indirect measure of product. (The fluorescent intensity of SYBR® Green is enhanced over 100-fold in binding to DNA.) PCR primers were designed so as to amplify all known splice-variants of each gene, and so that the size of all PCR products would be shorter than 150 base pairs in length, to accommodate the degraded, relatively shorter-length RNA expected to be found in FFPE samples. Primers used in the amplification of the 14 genes in the molecular signature described herein and three endogenous control genes are listed in Table 3. RT-PCR amplifications were performed in duplicate, in 384-well amplification plates. Each well contained a 15 ul reaction mix. The cycle profile consisted of: two minutes at 50° C., one minute at 95° C., 30 minutes at 60° C., followed by 45 cycles of 15 seconds at 95° C. and 30 seconds at 60° C., and ending with an amplification product dissociation analysis. The PCR components were essentially as described in L. Rogge, E. Bianchi et al., 2000, Nat Genet 25:96-101.

The relative changes in gene expression were determined by quantitative PCR. Expression levels were calculated by the Δ(ΔCₜ) method, where Ct=the threshold cycle for target amplification; e.g., the cycle number during PCR at which exponential amplification of the target nucleic acid begins. (KJ Livak and TD Schmittgen, 2001, Methods 25:402-408). The relative level of mRNA of a gene of interest was defined as:

Δ(ΔCt)=(Ct_{GOI}−Ct_{EC})_{test RNA}−(Ct_{GOI}−Ct_{EC})_{ref RNA}

where GOI=gene of interest, test RNA=sample RNA, ref RNA=calibrator reference RNA, and EC=endogenous control. The expression level of every gene of interest was first normalized to the three endogenous control genes. A Ct representing the average of the three endogenous controls (Ct_{EC}) was used to minimize the risk of normalization bias that would occur if only one control gene was used. (T. Suzuki, P J Higgins et al., 2000, Biotechniques 29:332-337). Primers used to amplify the endogenous controls are listed in Table 3. The adjusted expression level of the gene of interest was further normalized to a calibrator reference RNA pool, ref RNA (universal human reference RNA, Stratagene, La Jolla, Calif.). This was used in order to standardize expression results obtained from various machines. The Δ(ΔCt) values obtained in expression profiling experiments of 200 genes were used in the statistical analysis described below to determine the 14-gene prognostic signature.

### Determination of the 14-Gene Signature

Using data from expression profiling of the original 200 genes (i.e., the Δ(ΔCt) values obtained above), a semi-supervised principal component (SPC) method of determining survival time to distant metastasis was used to develop a list of genes that would comprise a prognostic signature. (E. Bair and R. Tibshirani, 2004, PloS Biology 2:0511-0522). SPC computation was performed using the PAM application, available online via the lab of R. Tibshirani at Standford University, Stanford, Calif. according to the method of R. Tibshirani, T J Hastie et al. 2002, PNAS 99:6567-6742.

Genes were first ranked according to their association with distant metastasis, using the univariate Cox proportional hazards model. Those genes with a P value <0.05 were considered significant. For any cutoff in the Cox score, SPC computed the component of genes (i.e., the principal component) that reached the optimal threshold: SPC used internal cross-validation in conjunction with a Cox model (with the principal component as the single variable) to select the optimal threshold. The first principal component gene list obtained by SPC was significant, and was used as a predictor in a univariate Cox model, in order to determine the correct sign and scaling.

The principal component gene list as produced by SPC was further reduced by the Lasso regression method. (R. Tibshirani, 1996, J Royal Statistical Soc B, 58:267-288). The Lasso regression was performed using the LARS algorithm. (B. Efron, T. Hastie et al., 2004, Annals of Statistics 32:407-499; T. Hastie, R. Tibshirani et al., eds., 2002, The Elements of Statistical Learning, Springer, New York). The outcome variable used in the LARS algorithm was the principal component as selected by SPC. The Lasso method selected a subset of genes that could reproduce this score with a pre-specified accuracy.

### Metastasis Score

The exemplary metastasis score (MS) has the form:

$$M S = a 0 + b * {\sum }_{i = 1}^{M} ai * Gi$$

Gi represents the standardized expression level of each Lasso-derived gene (i) of the 14-gene prognostic signature. The value of Gi is calculated from subtracting the mean gene expression of that gene in the whole population from the Δ(ΔCt) obtained in expression profiling described above and then divided by the standard deviation of that gene. The constant ai are the loadings on the first principal component of the fourteen genes listed in Table 2. An exemplary ai value for each gene i is provided in Table 2. In this example, the constant b was −0.251 and it was from a univariate Cox model with the principal component as a predictor, to get the correct sign and scaling. The constant a0=0.022 (as an example value); this centers the MS so that its median value is zero. M is the number of genes in the component list; in this case fourteen. Thus, in certain exemplary embodiments, the MS is a measure of the summation of expression levels for the 14 genes disclosed in Table 2, each optionally multiplied by a particular constant ai (or equally weighted); the summation can then optionally be multiplied by the constant b and this summation can optionally be added to a centering constant (for example, 0.022).

The score is herein referred to as MS (all), as it was based on an analysis of all 142 ER-positive individuals studied. A sample from a breast cancer patient can be evaluated by generating this metastasis score from the 14-gene expression profiling data for that patient, and the probability of distant metastasis and/or the predicted response to breast cancer chemotherapy can be determined for the patient.

Generation of Distant Metastasis Probability from MS

The probability of distant metastasis for any individual patient can be calculated from the MS at variable time points, using the Weibull distribution as the baseline survival function.

The metastasis score (MS) obtained above, from expression profiling of the 14-gene signature, was converted into the probability of distant metastasis by means of the Cox proportional hazard model. Because the Cox model does not specify the baseline hazard function, the hazard and survivor functions were first constructed through parametric regression models. In the parametric regression models, distant metastasis-free survival time was the outcome, and the metastasis score (MS) was the independent variable input. The event time was assumed to have a Weibull distribution; its two parameters were estimated using the survival data from which the MS was derived. To calculate the probability of distant metastasis within a certain time for a patient, the MS value is simply substituted into the formula for the survivor function.

### Pre-Validation

In this study, the 142 study patients were randomly divided into ten subsets. One subset was set aside and the entire SPC procedure was performed on the union of the remaining nine subsets. Genes were selected and the prognosticator built upon the nine subsets was applied to obtain the cross-validated metastasis score, referred to herein as “MS (CV)”, for the remaining subset. This cross-validation procedure was carried out 10 times until MS (CV) was filled in for all patients. By building up MS (CV) in this way, each 1/10^{th} piece did not directly use its corresponding survival times, and hence can be considered unsupervised.

This resulted in a derived variable for all the individuals in the sample, and could then be tested for its performance and compared with other clinical variables. MS (all), however, was built upon all 142 individuals tested, and would produce considerable bias if tested in the same way.

MS (CV) was used to evaluate the accuracy of the 14-gene prognostic signature when time-dependent area under ROC curve (AUC) was calculated (described below). MS (CV) was also used in the Cox regression models when the 14-gene signature was combined with clinical predictors. MS (CV) should have one degree of freedom, in contrast to the usual (non-pre-validated) predictor. The non-pre-validated predictor has many more degrees of freedom.

### Statistical Analyses of the MS

Kaplan-Meier (KM) curves for distant metastasis-free and overall survival were generated for the high- and low-risk patient groups using the median of MS (CV) as the cut point (i.e., 50 percentile of MS (CV)). The choice of median MS as cut point was based upon the calculation of the hazard ratios of the high vs. low-risk groups using different cut points from ten percentile of MS to ninety percentile of MS. A balanced number of high-risk and low-risk individuals as well as near the highest hazard ratio were the determining factors for choosing the median as the cut point.

Hazard ratios (HR) and 95% confidence intervals (CI) using the cut point of median MS were calculated and reported. Log rank tests were performed, and the hazard ratios were calculated for different cut points. The accuracy and value of the 14-gene signature in predicting distant metastasis at five years were assessed by various means. (XH Zhou, N. Obuchowski et al., eds., 2002, Statistical Methods in Diagnostic Medicine, Wiley-Interscience, New York).

Univariate and multivariate Cox proportional hazards regressions were performed using age, tumor size, tumor grade and the 14-gene signature. Clinical subgroup analyses on the signature were also performed. Statistical analyses were performed using SAS® 9.1 statistical software (SAS Institute, Inc., Cary, N.C.), except for the statistical packages noted herein.

### Multi-Gene Signature

Of the 200 candidate genes studied, 44 had unadjusted P values <0.05 in a univariate Cox proportional hazards regression. Patients with poor metastasis prognosis showed an up-regulation of 37 genes, while seven genes were down-regulated. The semi-supervised principal component procedure (SPC) in PAM yielded a prognosticator of 38 genes. The gene list was further reduced to 14 (Table 2) by using the Lasso regression, via the LARS algorithm. Table 2 provides a description of each gene. Hazard ratios (HR) at various cut points (i.e., percentile MS (CV)) were calculated. The median of MS (CV) was chosen to classify patients into low- and high-risk groups.

### Results

### Distant-Metastasis-Free and Overall Survival Rates in Low-Risk and High-Risk Groups

There were 7 and 24 distant metastases in 71 low-risk and 71 high-risk patients as defined by the median of the cross-validated metastasis score, MS (CV), in the training set. Kaplan Meier estimate (FIG. 1a) indicated significant differences in distant metastasis free survival (DMFS) between the two groups with a log-rank p-value of 0.00028. The 5-year and 10-year DMFS rates (standard error) in the low-risk groups were 0.96 (0.025) and 0.90 (0.037) respectively. For the high-risk group, the corresponding rates were 0.74 (0.053) and 0.62 (0.066). For overall survival (FIG. 1b), there was also significant difference between the two groups (log-rank p-value=0.0048). The 5-year and 10-year OS rates (standard error) in the low-risk groups were 0.90 (0.036) and 0.78 (0.059) while the corresponding rates were 0.79 (0.049) and 0.48 (0.070) in the high-risk group (Table 5).

Hazard Ratios from Univariate and Multivariate Cox Regression Models

The unadjusted hazard ratio of the high-risk vs. low-risk groups by MS to predict DMFS was 4.23 (95% CI=1.82 to 9.85) (Table 6) as indicated by the univariate Cox regression analysis. In comparison, the high-risk vs. low-risk groups by tumor grade (medium+high grade vs. low grade) had an unadjusted hazard ratio of 2.18 (1.04-4.59). While tumor size was significant in predicting DMFS (p=0.05) with 7% increase in hazard per cm increase in diameter, age was not a significant factor in this patient set. In the multivariate Cox regression analyses, the 14-gene molecular signature risk group had a hazard ratio of 3.26 (1.26-8.38), adjusted by age at surgery, tumor size and grade. It was the only significant risk factor (p=0.014) in the multivariate analyses.

### Diagnostic Accuracy and Predictive Values

Diagnostic accuracy and predictive values of the 14-gene signature risk groups to predict distant metastases within 5 years were summarized in Table 9. Sensitivity was 0.86 for those who had distant metastases within 5 years while specificity was 0.57 for those who did not. Negative predictive value (NPV) was 96% and indicated that only 4% of individuals would have distant metastasis within 5 years when the gene signature indicated that she was in the low-risk group. Nevertheless, positive predictive value (PPV) was only 26%, indicating only 26% of individuals would develop distant metastasis while the molecular signature indicated she was high-risk. The high NPV and low PPV were partly attributed to the low prevalence of distant metastasis in 5 years, which was estimated to be 0.15 in the current patient set.

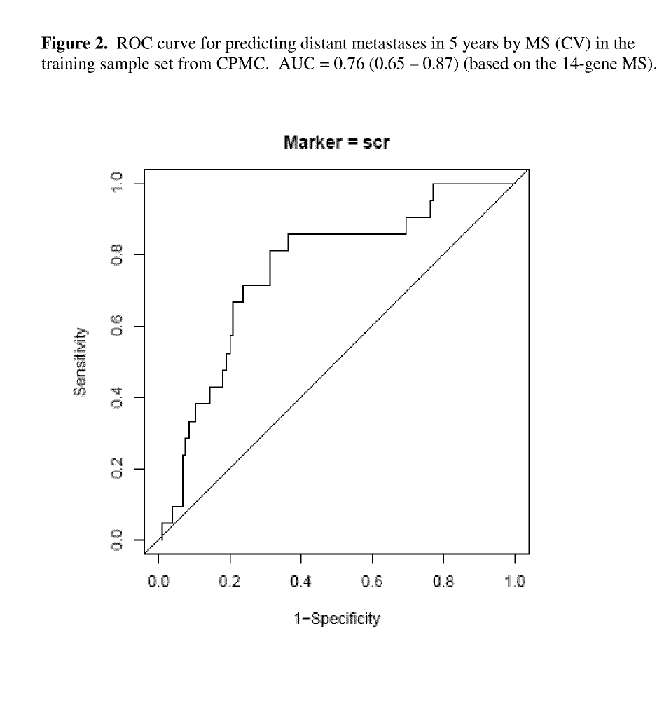

Moreover, receiver operating characteristics (ROC) curves of the continuous MS (CV) to predict distant metastases in 5 years is shown in FIG. 2. AUC was 0.76 (0.65-0.87) for predicting distant metastases in 5 years. AUC for predicting death in 10 years was 0.61 (0.49-0.73). One sided tests for AUC to be greater than 0.5 were significant with corresponding p-values of <0.0001 and 0.04 for the metastasis and death endpoints.

### Discussion

Pathway analyses revealed that the fourteen genes in the prognostic signature disclosed herein are involved in a variety of biological functions, but a majority of the genes are involved with cell proliferation. Eleven of the fourteen genes are associated with the TP53 and TNF signaling pathways that have been found to be coordinately over-expressed in tumors leading to poor outcome. BUB1, CCNB1, MYBL2, PKMYT1, PRR11 and ORC6L are cell cycle-associated genes. DIAPH is a gene involved in actin cytoskeleton organization and biogenesis. DC13 is expected to be involved with the assembly of cytochrome oxidase.

Whereas previously reported studies were limited to the use of frozen tissues as the source of RNA and were profiled on microarrays, the invention described in this example demonstrates that real-time RT-PCR may be used for gene profiling in FFPE tumor samples. Thus, it provides for the use of archived breast cancer tissue sections from patients who have extended-outcomes data that predate the routine use of adjuvant therapy.

Distant metastasis-free survival is the prognostic endpoint for the study described in this example. A supervised principal components (SPC) method was used to build the 14-gene signature panel of exemplary embodiments of the invention. The approach used in assembling the signature allowed the derivation of a metastasis score (MS) that can translate an individual's expression profile into a measure of risk of distant metastasis, for any given time period. The ability to quantify risk of metastasis for any timeframe provides highly flexible prognosis information for patients and clinicians in making treatment decisions, because the risk tolerance and time horizon varies among patients.

With the 14-gene molecular signature, high and low-risk groups had significant differences in distant metastasis-free and overall survival rates. This signature includes proliferation genes not routinely tested in breast cancer prognostics. The 14-gene signature has a ten gene overlap with the 50-gene signature described by H. Dai, L J van't Veer et al. (2005, in Cancer Res 15:4059-4066). In contrast, only six genes overlap with the 70-gene signature described by Dai, van't Veer et al. (2002, Nature 415:530-536). This may be explained by the fact that that study analyzed a more heterogeneous group of patients, which included both ER-positive and negative patients. The signature described herein had two proliferation gene overlaps with the 16-gene signature described by SP Paik, S. Shak et al., (2004, N Engl J Med 351:2817-2826).

The molecular signature described herein has independent prognostic value over traditional risk factors such as age, tumor size and grade, as indicated from multivariate analyses. This signature provides an even more compelling measure of prognosis when the tumor grade is low. As reported by Dai et al., a subset of this patient group with low grade tumors may be at even higher risk of metastasis than previously estimated. (Dai et al., 2005, Cancer Res 15:4059-4066). The signature described herein also extends the confidence in the prognostic genes initially reported by van't Veer et al. (2002, Nature 415:530-536) and Dai et al. (2005, Cancer Res 15:4059-4066), who primarily used samples from women less than 55 years of age, because this signature was validated on patients with a broad age distribution (median 64 years old), which is similar to the general range of breast cancer patients.

The use of FFPE tissues to sample even smaller amounts of sectioned tumor than microarray studies using frozen tissue, corroborated a subset of the genes on a different detection platform (quantitative PCR versus microarrays). This reiteration of results is consistent with the concept described by Bernards and Weinberg, that metastatic potential is an inherent characteristic of most of, rather than a small fraction of, the cells in a tumor. (R. Bernards and RA Weinberg, 2002, Nature 418:823).

Exemplary embodiments of the invention described herein also provide an objective estimate of prognosticator performance by using the pre-validation technique proposed by Tibshirani and Efron. (R. Tibshirani, B. Efron, 2002, Statistical Applications in Genetics and Molecular Biology 1:article 1). Several investigators have noted the importance of independent validation in increasingly large and characterized datasets (R. Simon, J Clin Oncol, 2005, 23:7332-7341; DF Ransohoff, 2004, Nat Rev Cancer 4:309-14; DF Hayes, B. Trock et al., 1998, Breast Cancer Res 52: 305-319; DG Altman and P. Royston, 2000, Stat Med 19:453-73).

In exemplary embodiments of the invention, a unique 14-gene prognostic signature (or subsets thereof) is described that provides distinct information to conventional markers and tools and is not confounded with systemic treatment. While the signature was developed using FFPE sections and RT-PCR for early stage, node-negative, ER-positive patients, it may be used in conjunction with any method known in the art to measure mRNA expression of the genes in the signature and mRNA obtained from any tumor tissue source, including but not limited to, FFPE sections, frozen tumor tissues and fresh tumor biopsies. Based on the mRNA expression levels of the 14 genes disclosed herein (or a subset thereof), a metastasis score can be calculated for quantifying distant metastasis risk for a breast cancer patient. Thus, the various embodiments of the invention disclosed herein are amenable for use in routine clinical laboratory testing of ER-positive breast cancer patients for any timeframe.

### Example Two

### The 14-Gene Signature Predicts Distant Metastasis in Untreated Node-Negative, ER-Positive Breast Cancer Patients Using 280 FFPE Samples

Efforts were undertaken to validate the 14-gene expression signature that can predict distant metastasis in node-negative (N−), estrogen receptor positive (ER+) breast cancer patients in an independent sample set who had not received systemic treatment. Reference is made to the experimental protocols and statistical analyses in Example 1, which were used to assay the effectiveness of the 14-gene signature.

### Patients & Methods

A retrospective search of the Breast Tissue and Data Bank at Guy's Hospital was made to identify a cohort of patients diagnosed with primary breast cancer and who had definitive local therapy (breast conservation therapy or mastectomy) but no additional adjuvant systemic treatment. The study group was restricted to women diagnosed between 1975 and 2001, with a clinical tumor size of 3 cm or less, pathologically uninvolved axillary lymph nodes, ER-positive tumor and with more than 5 years follow-up. A total of 412 patients were identified who also had sufficient formalin fixed, paraffin embedded (FFPE) tissues available for RNA extraction. The use of patient material and data for this study has been approved by Guy's Research Ethics Committee.

From this group there was sufficient quantity and quality of mRNA to profile tumors from 300 patients. Subsequently a further 20 cases were excluded from the study. Six patients had bilateral breast cancer prior to distant metastasis, 6 had a missing value in gene expression levels and 8 tumors proved to be ER negative upon re-assessment using current techniques. Thus, in total 280 patients were included in the analyses (validation set from Guy's Hospital in Table 1). To assess selection bias, the 280 patients who were analyzed were compared with the 412 patients who were identified to satisfy the inclusion criteria and had sufficient FFPE tissues for RNA extraction. No selection bias was detected as there were no significant differences in age, tumor size and histologic grade between the two sets.

ER status on this group of patients had been re-evaluated using contemporary IHC assay. Allred score 3 or more were considered receptor positive. Tumors were classified according to WHO guidelines (World Health Organization, Geneva, Switzerland. Histological typing of breast tumours. Tumori 1982; 68:181), and histological grade established using the modified Bloom and Richardson method (Elston C W & Ellis I O. Pathological prognostic factors in breast cancer. I. The value of histological grade in breast cancer: experience from a large study with long term follow-up. Histopathology 1991; 19:403-10).

Compared with the training set, the validation set was younger, with larger tumors and with a larger proportion of high grade tumors (Table 1). Tests for differences in those characteristics are highly significant (p<0.001).

### Metastasis Score

MS in Equation 1 derived in the training samples in Example 1 was applied to the untreated patients from Guy's Hospital. In Example 1, RNA had been enriched, whereas in the case of untreated patients from Guy's Hospital, the RNA samples were not enriched.

To apply MS to the un-enriched samples, conversion factors between enriched and un-enriched samples were obtained from 93 training samples in Example 1 for each of the genes in the molecular signature.

### Results

### Distant Metastasis Free and Overall Survival Rates in Low-Risk and High-Risk Groups

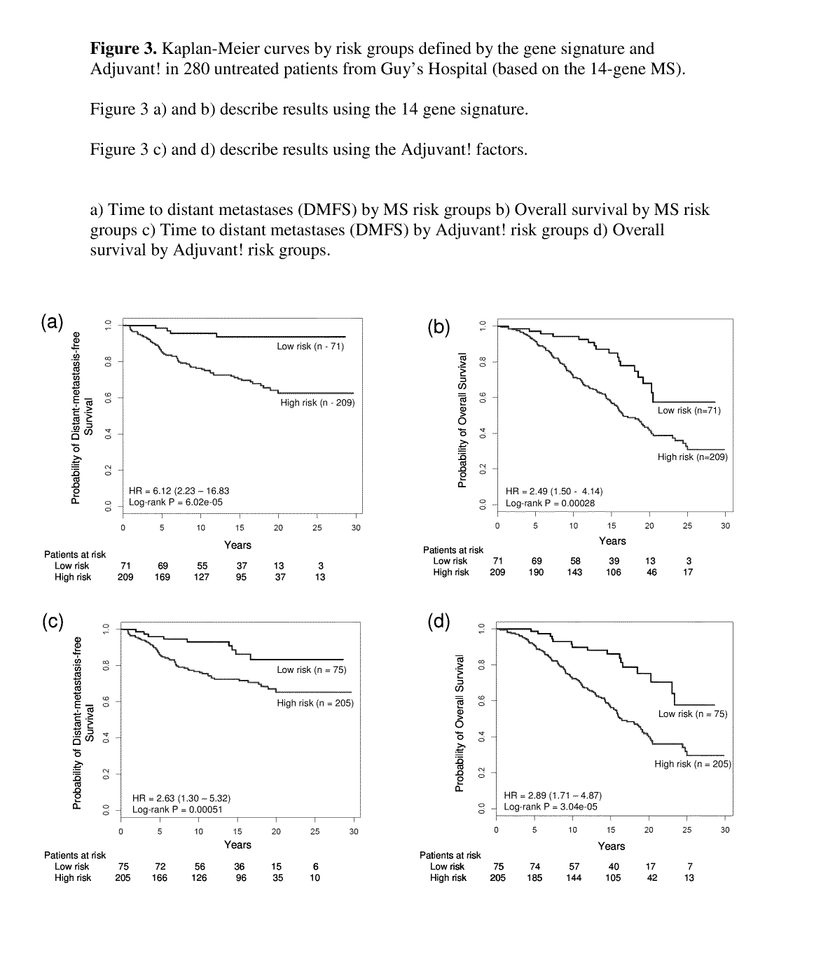

There were 4 (5.6%) distant metastases in the 71 MS low-risk and 62 (29.7%) distant metastases in the 209 MS high-risk patients, respectively. Kaplan Meier estimate (FIG. 3a) indicated significant differences in DMFS between the two groups with a log-rank p-value of 6.02e-5. The 5-year and 10-year DMFS rates (standard error) in the low-risk group were 0.99 (0.014) and 0.96 (0.025) respectively. For the high-risk group, the corresponding survival rates were 0.86 (0.025) and 0.76 (0.031) (Table 12).

For the Adjuvant! risk groups, Kaplan Meier curves of DMFS were shown in FIG. 3c. The 5-year and 10-year DMFS rates (standard error) were 0.96 (0.023) and 0.93 (0.03) for the low-risk group and 0.87 (0.03) and 0.77 (0.031) for the high-risk group, respectively. There were larger differences in survival rates between the high-risk and low-risk groups defined by MS than those defined by Adjuvant!.

For overall survival (OS), Kaplan Meier curves (FIG. 3b) indicated significant difference in OS rates between MS low-risk and high-risk groups (log-rank p-value=0.00028). The 5-year and 10-year OS rates were 0.97 (0.020) and 0.94 (0.028) for the low-risk group and 0.92 (0.019) and 0.71 (0.032) for the high-risk group, respectively. FIG. 3d showed the Kaplan Meier curves for Adjuvant! to predict 5-year and 10-year overall survival. MS and Adjuvant! provide similar prognostic information for overall survival.

Hazard Ratios from Univariate and Multivariate Cox Regression Models

The unadjusted hazard ratio of the high-risk vs. low-risk groups by MS to predict time to distant metastases was 6.12 (95% CI=2.23 to 16.83) (Table 10). The unadjusted hazard ratio for MS risk groups is higher than those for groups defined by Adjuvant!, age, tumor size and histologic grade. Adjuvant! had the second highest hazard ratio of 2.63 (95% CI 1.30-5.32). Risk groups by histologic grade and tumor size were significant in predicting DMFS, but not the age group.

Age group is the most significant prognostic factor in predicting OS with an unadjusted hazard ratio of 2.9 (95% CI 2.03-4.18) (Table 10). Nevertheless, MS risk group can predict overall survival with HR of 2.49.

In the multivariate Cox regression (Table 11a) of time to distant metastases with the MS risk group and clinicopathological risk factors of age, tumor size and histological grade, the hazard ratio of the MS risk group, adjusted by age, tumor size and histologic grade, is 4.81 (1.71-13.53, p=0.003). MS risk group was the only significant risk factor in the multivariate analysis. Therefore, the gene signature has independent prognostic value for DMFS over the traditional clinicopathological risk factors and captures part of information within these factors.

In the multivariate Cox regression of time to distant metastases with the risk groups by the 14-gene signature and by Adjuvant! (Table 11b), the corresponding adjusted hazard ratios were 5.32 (1.92-14.73) and 2.06 (1.02-4.19). Both MS and Adjuvant! risk groups remained significant to predict DMFS. This indicates that MS and Adjuvant! carried largely independent and complementary prognostic information to each other.

### Performance of the Molecular Signature in Different Clinical Subgroups

Table 13 shows that the gene signature predicts distant metastasis in young and old, pre-menopausal and post-menopausal women. While highly prognostic in patients with small size tumors (HR=14.16, p=0.009), it is not significant in patients with tumors larger than 2 cm. While hazard ratio in the low-grade subgroup (HR=7.6) is higher than that in the high-grade (HR=4.6), it only shows a trend to significance (p=0.06) in the low-grade subgroup because of small sample size (7 events in 60 samples).

Hazard ratios in various subgroups indicated that the gene signature is more prognostic in low grade, small size tumors, young and pre-menopausal patients in the validation sample set. Formal tests for interaction between the MS risk group and the clinical variables were not significant. However, the signature was also more prognostic in the low-grade tumors in the CPMC training set. Nevertheless, interaction analyses should be regarded as exploratory as multiple tests were performed.

### Diagnostic Accuracy and Predictive Values

The diagnostic accuracy and predictive values of the risk groups by MS and Adjuvant! to predict distant metastases in 10 years were shown in Table 14. The MS risk group has higher sensitivity of 0.94 (0.84-0.98) than the Adjuvant! risk group's 0.90 (0.78-0.96) while the specificity is similar (0.3 (0.24-0.37) for MS vs. 0.31 (0.26-0.38) for Adjuvant!). Using 0.18 as the estimated prevalence of distant metastases in 10 years, PPV and NPV for MS risk group were 0.23 (0.21-0.25) and 0.97 (0.88-0.99) respectively. The corresponding values were 0.23 (0.20-0.25) and 0.93 (0.85-0.97) for the Adjuvant! risk group. Therefore, MS can slightly better predict those who would not have distant metastases within 10 years than Adjuvant! while the predictive values for those who would have distant metastases within 10 years were similar for the molecular and clinical prognosticators.

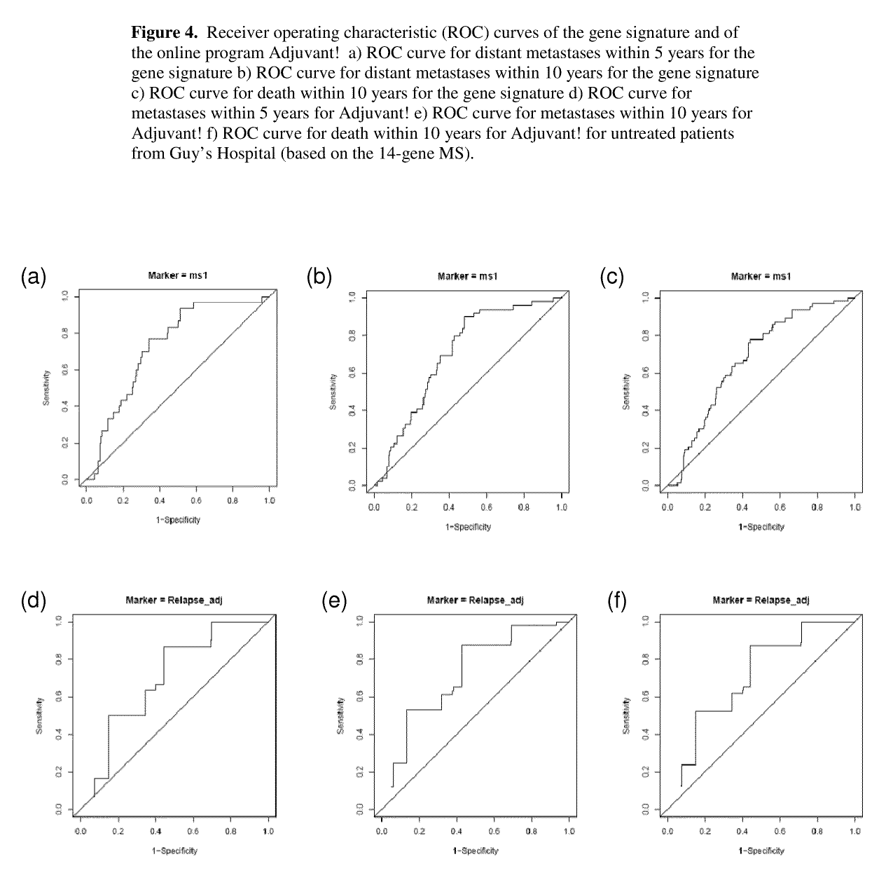

ROC curves (FIG. 4) of continuous MS to predict distant metastasis within 5 years and 10 years had AUC (95% CI) of 0.73 (0.65-0.81), 0.70 (0.63-0.78). A ROC curve to predict death in 10 years had an AUC of 0.68 (0.61-0.75). Hence, MS are predictive of both distant metastases and deaths.

In comparison, AUCs of ROC curves to predict distant metastases within 5 years, 10 years and death in 10 years by Adjuvant! were 0.63 (0.53-0.72), 0.65 (0.57-0.73) and 0.63 (0.56-0.71) and they were lower than the corresponding values by MS.

### MS as a Continuous Predictor of Probability of Distant Metastasis

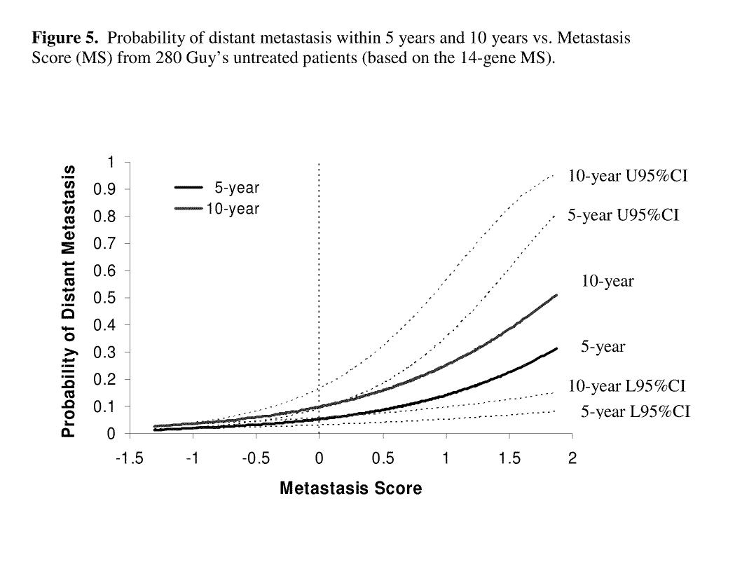

FIG. 5 shows the probabilities of distant metastasis at 5 and 10 years for an individual patient with a metastasis score, MS. Five-year and ten-year distant metastasis probabilities have median (min-max) of 8.2% (1.4%-31.2%) and 15.2% (2.7%-50.9%) respectively. At zero MS, the cut point to define the risk groups, the 5-year and 10-year distant metastasis probabilities were 5% and 10%, respectively.

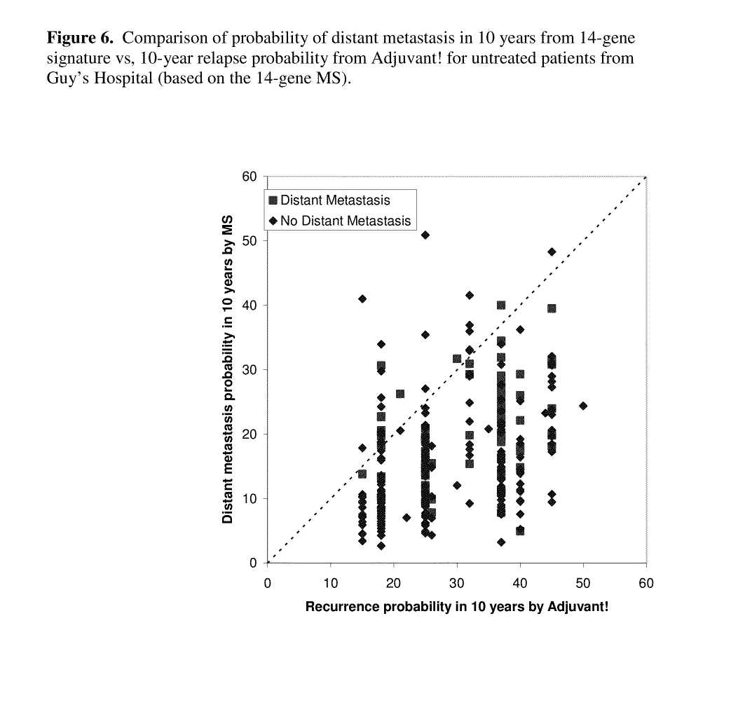

The probability of distant metastasis in 10 years by MS was compared with the probability of relapse in 10 years by Adjuvant! (FIG. 6). The coefficient of determination (R²) was 0.15 indicating that only a small portion of variability in probability of distant metastasis by MS can be explained by Adjuvant! The probability of distant metastasis by MS was lower than the relapse probability by Adjuvant! as all recurrence events were included in the Adjuvant! relapse probability while only distant metastases were counted as an event in the MS estimate of probability of distant metastasis.

### Discussion

Initially a 14-gene prognostic signature was developed based upon mRNA expression from FFPE sections using quantitative RT-PCR for distant metastasis in a node-negative, ER-positive, early-stage, untreated breast cancer training set. The resulting signature was used to generate a metastasis score (MS) that quantifies risk for individuals at different timeframes and was used to dichotomize the sample set into high and low risk. Following initial internal validation of training set using a “pre-validation” statistical technique, the expression signature was validated using the precise dichotomized cutoff of the training set in a similar and independent validation cohort. Performance characteristics of the signature in training and validation sets were similar. Univariate and multivariate hazard ratios were 6.12 and 4.81 for the validation set and 4.23 and 3.26 in the training set to predict DMFS, respectively. In multivariate analysis, only the metastasis score remained significant with a trend to significance for only tumor size of the other clinicopathological factors. The 14-gene prognostic signature can also predict overall survival with univariate hazard ratios of 2.49 in the validation set. ROC curves of continuous MS to predict distant metastasis within 5 and 10 years and to predict death in 10 years had AUC of 0.73, 0.70 and 0.68, respectively. The signature provided more compelling prognosis when the tumor grade was low (hazard ratio were 7.58 in the low grade and 4.59 in the high grade tumors). Dai et al, for example, interpreted the change in prognostic power of the classifier as not being reflective of a continuum of patients but instead differential performance in discrete groups of patients.

When compared to risk calculated from Adjuvant!, a web-based decision aid, there was only a modest correlation with MS. In multivariate Cox regression, MS and Adjuvant! risk groups both remain significant prognostic factors when they are adjusted for each other. There were larger differences in survival rates between the high-risk and low-risk groups defined by MS relative to Adjuvant!. MS can better predict those who would not have distant metastases within 10 years than Adjuvant!. These data demonstrate that the molecular signature provides information that is independent from other prognostic tools being adopted for predicting breast cancer distant metastasis.

### Example Three

### The 14-Gene Signature Predicts Distant Metastasis in Both Treated and Untreated Node-Negative and ER-Positive Breast Cancer Patients Using 96 FFPE Samples

Efforts were undertaken to validate a 14-gene expression signature that can predict distant metastasis in node negative (N−), estrogen receptor positive (ER+) breast cancer patients in an independent sample set having both treated and untreated patients. Reference is made to the experimental protocols and statistical analyses in Example 1, which were used to assay and evaluate the effectiveness of the 14-gene signature.

### Patients & Methods

A cohort of 96 N−, ER+ breast cancer patients, with a mean age of 56.7 years, was selected for the validation study (Table 15). The patients in the validation study were selected from University of Muenster. Of those, 15 were untreated, 54 were treated with Tamoxifen alone, 6 were treated with chemotherapy alone, and 2 were treated with both Tamoxifen and chemotherapy. Nineteen patients had unknown treatment status. The fourteen genes in the signature were profiled in FFPE samples using quantitative RT-PCR. A previously derived metastasis score (MS) was calculated for the validation set from the gene expression levels. Patients were stratified into two groups using a pre-determined MS cut point, which was zero.

### Validation of Metastasis Score

MS in Equation 1 that was derived with samples in Example 1 was applied to the patients from University of Muenster. In this example, RNA from the tumor tissues was also enriched but to a lesser extent than those in Example 1. To apply MS to this example, conversion factors between enriched and un-enriched samples were obtained from 93 samples from University of Muenster for each of the 14 genes in the signature. The conversion factors between enriched and unenriched samples were also obtained from 93 training samples from CPMC in Example 1. The conversion factors between the gene expression levels from CPMC and University of Muenster were then calculated using those two sets of conversion factors.

### Results

### Distant-Metastasis-Free Survival Rates

Using MS zero as cut point, patients were classified as high-risk and low-risk. Of all 96 patients, 48 patients were identified as high-risk with a 5-year DMF survival rate (standard error) of 0.61 (0.072) while 48 low-risk patients had a corresponding survival rate of 0.88 (0.052). Of the 62 treated patients, 32 high-risk and 30 low-risk patients had 5-yr DMF survival rates of 0.66 (0.084) and 0.89 (0.060), respectively. Of the 54 patients who received Tamoxifen treatment alone, 26 high-risk and 28 low-risk patients had 5-yr DMF survival rates of 0.65 (0.094) and 0.88 (0.065) respectively (Table 16).

### Unadjusted Hazard Ratios

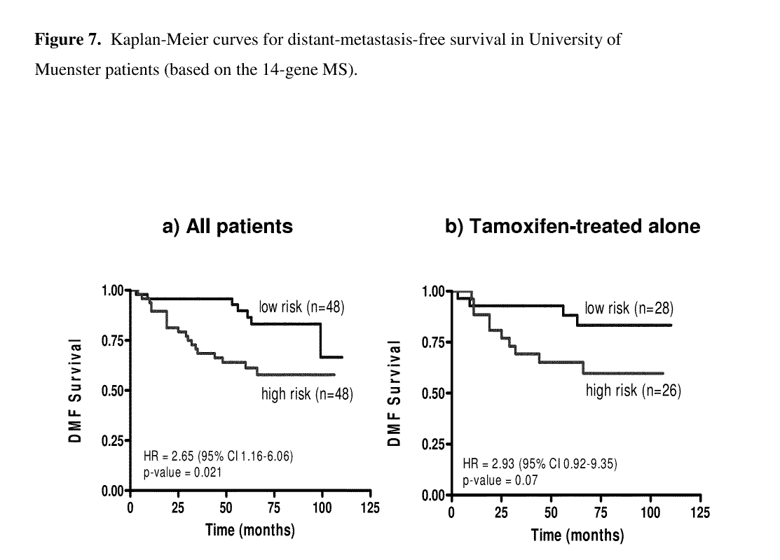

For the entire cohort, the MS correlated with distant metastasis-free (DMF) survival. Cox proportional hazard regression indicated 2.67 (1.28-5.57) times increased hazard per unit increase of MS (p=0.0087). Using zero as cut point, The hazard ratio of high-risk vs. low-risk patients in the entire cohort was 2.65 (1.16-6.06, p=0.021). For 61 treated patients, the hazard ratio was 3.08 (0.99-9.56, p=0.052). For the 54 patients who were treated with Tamoxifen only, the hazard ratio was 2.93 (0.92-9.35, p=0.07). Survival rates and hazard ratios for different groups of patients are summarized in Table 16 and FIG. 7, respectively.

An RT-PCR based 14-gene signature, originally derived from untreated patients, can predict distant metastasis in N−, ER+, Tamoxifen-treated patients in an independent sample set using FFPE tissues. There was a large differential DMFS rates between high and low risk Tamoxifen-treated alone patients (0.65 vs. 0.88) where two groups were defined by MS using zero as cut point. Differential risk between top and bottom quintiles of multi-modal MS were 3.99 and 3.75 fold for all and Tamoxifen-treated alone patients, respectively. The prognostic signature may provide baseline risk that is not confounded with systemic treatment. Moreover, it can predict metastatic risk for patients who receive treatment. Therefore, the gene signature would be applicable in identifying women with a poor clinical outcome to guide treatment decisions, independent of the subsequent therapies.

### Example Four

### The 14-Gene Signature Predicts Distant Metastasis in Tamoxifen-Treated Node-Negative and ER-Positive Breast Cancer Patients Using 205 FFPE Samples

### Patients

A cohort of 205 women with N−, ER+ breast cancer who had surgery between 1975 and 2001 in Guy's Hospital was selected. The median follow-up was 9.3 years. Among them, there were 17 (8.9%) distant metastases, 44 deaths (21.5%) and 17 (8.9%) local and distant recurrences. 138 (67.3%) patients were at stage I while 67 (32.7%) were at stage II. All patients received adjuvant hormonal treatment but no chemotherapy. The cohort had a mean (SD) age of 59.3 (10.4) years. 64% were over the age of 55 years and 80.5% were post-menopausal. All tumors were <3 cm in diameter and the mean (SD) tumor diameter was 1.67 cm (1.0). 60 (29.3%), 98 (47.8%) and 47 (22.9%) patients had tumors of histological grade 1, 2 and 3, respectively. (Table 17)

### Endpoints

Time from surgery to distant metastasis, also referred to as distant-metastasis-free survival (DMFS), was chosen as the primary endpoint. Events were distant metastases. Contra-lateral recurrences and deaths without recurrence were censoring events while local recurrences were not considered events or censoring events. The definition of DMFS endpoint, its events and censoring rules were aligned with those adopted by the National Surgical Adjuvant Breast and Bowel Project (NSABP) for the prognostic molecular marker studies (Paik et al 2004). The DMFS endpoint is most directly linked to cancer related death.

### Gene Expression Signature and Metastasis Score (MS)

A 14-gene signature was previously developed using profiling study by RT-PCR with FFPE samples from California Pacific Medical Center (CPMC) as described in Example 1. Pathway analyses by the program Ingenuity revealed that the majority of the 14 genes in the signature are involved with cell proliferation. Ten of 14 genes are associated with TP53 signaling pathways that have been found to be coordinately over-expressed in tumors of poor-outcome.

A metastasis score (MS) was calculated for each individual. MS in this example was based upon the gene expression of the 14 genes in the signature as previously described. However, the algorithm for calculating MS in this example was different from the algorithm described for the previous examples. Nevertheless, MS derived with the new algorithm was highly correlated with MS derived with the previous method with Pearson correlation coefficient >0.99. Moreover, in this example, two cut points were employed to group patients into high, intermediate and low MS groups as opposed to using only one cut point to categorize patients into low and high risk groups in the previous three examples.

While the 14 genes in the signature were chosen in the study as described in Example 1, the new MS score and cut points were determined based upon the study using untreated samples from Guy's Hospital as described in Example 2. The new MS algorithm was applied and validated in Examples 4 and 5. The new metastasis score (MS(new)) is now calculated as the negative of the mean of the gene expression level of 14 genes. With this new score, the fourteen genes were given equal weighting. The −1 multiplier was used so that higher MS corresponds to higher risk of distant metastasis. The new MS can be expressed in the following formula:

$$M S ( new ) = - ( 1 / 14 ) * [ {\sum }_{i = 1}^{14} Gi ] \qquad Equation 3$$

where Gi are the centered expression levels of the 14 genes in the signature.

Two cut points of MS(new) were chosen to categorize patients into high, intermediate and low MS groups. The lower cut point was −1.47 while the upper cut point was −0.843. Individuals with MS smaller than −1.47 were in the low MS group. Individuals with MS between −1.47 and −0.843 were in the intermediate MS group while those with MS greater than −0.843 were in the high MS group. If those with low MS were considered low-risk while those in intermediate MS and high MS groups were considered as high risk in Guy's untreated samples (in other word those with MS above −1.47 were considered high risk), then sensitivity of the MS risk groups would be above 90%. On the other hand, if those with low MS and intermediate MS were considered low-risk while those with high MS were considered high-risk (in other words, those with MS lower than −0.843 were considered low-risk while those with MS higher than −0.843 were considered high-risk), then the sensitivity and specificity of the MS risk groups in Guy's untreated samples would be the same at 70%.

For the untreated samples, the intermediate MS group has risk similar to that of high MS group and the high and intermediate MS groups had higher risk than those with low MS. However, for patients treated with hormonal treatment, the intermediate MS group has risk similar to that of the low MS group. The risk of high MS group is higher than the risk of intermediate MS and low MS groups.

Another method of applying the 14 gene signature is by using Equation 2, as follows. A 14-gene signature was previously developed using profiling study by RT-PCR with FFPE samples from California Pacific Medical Center (CPMC) as described in Example 1. Pathway analyses by the program Ingenuity revealed that the majority of the 14 genes in the signature are involved with cell proliferation. Ten of 14 genes are associated with TP53 signaling pathways that have been found to be coordinately over-expressed in tumors of poor-outcome.

A metastasis score (MS) was calculated for each individual. MS in this example was based upon the gene expression of the 14 genes in the signature as previously described. However, the algorithm for calculating MS in this example was based upon Equation 2 in which the fourteen genes were weighted equally. Moreover, in this example, two cut points were employed to group patients into high, intermediate and low MS groups as opposed to using only one cut point to categorize patients into low and high risk groups in the previous three examples. While the 14 genes in the signature were chosen in the study as described in Example 1, the new MS score and cut points were determined based upon the study using untreated samples from Guy's Hospital as described in Example 2. The new MS algorithm was applied and validated in Examples 4 and 5.

Two cut points of MS(new) were chosen to categorize patients into high, intermediate and low MS groups. Cut points were determined such that when individuals with MS above the first cut point of −0.119 were classified as high-risk individuals to have distant metastasis in 5 years, the sensitivity of the MS risk groups would be above 90%. The second cut point of MS=0.302 was chosen such that sensitivity and specificity would be the same at 0.7.

It should be noted that the MS determined in Equation 2 and MS as the negative of the mean of gene expression of all fourteen genes are simply linear transformation of each other. As such they have perfect correlation (Pearson correlation coefficient=1) and the classification of patients into high, intermediate, and low MS are the same using the corresponding cut points as described.

### Statistical Analyses

Kaplan-Meier (KM) curves for distant metastasis free survival were generated for the high, intermediate and low MS groups. Upon examining the DMFS rates of the three groups, intermediate and low MS groups were combined as a low-risk group which was compared with the high risk group with high MS. Log rank tests were performed.

Univariate and multivariate Cox proportional hazard regression analyses of MS groups for DMFS endpoint were performed. Hazard ratio of high-risk (high MS) vs. low-risk (intermediate and low MS groups combined) group was adjusted for age (in years), tumor size (in cm) and histological grade in multivariate analysis.

Association of MS groups with age, tumor size was investigated using ANOVA tests while association of MS groups with histological grade was evaluated by Crammer's V for strength of association and by chi-sq tests for statistical significance.

Hazard ratios of the MS risk groups were also calculated for different clinical subgroups. Those groups include pre-menopausal vs. post-menopausal, age ≦55 years vs. >55 years, tumor size >2 cm vs. ≦2 cm, and histological grade 1 & 2 vs. grade 3.

To assess diagnostic accuracy, Receiver Operator Characteristic (ROC) curve of MS to predict distant metastases within 5 years was plotted. Area under the ROC curves (AUC) was calculated. Sensitivity, specificity, positive predictive value (PPV) and negative predictive value (NPV) were calculated with 95% confidence interval (95% CI) for high vs. low-risk groups by MS.

The time-dependence of hazard ratio of MS groups was investigated by estimating the annualized hazards using a spline-curve fitting technique that can handle censored data. The HEFT procedure in R2.4.1 was employed. Annualized hazards were estimated for both MS high and low-risk groups and from which, the hazard ratios at different time were calculated.

Kaplan Meier estimates and Cox proportional hazard regression were performed using R2.41.1 and SAS 9.1. ROC curves and AUC were estimated using the Mayo Clinic's ROC program. The Delong method of estimating confidence interval of AUC was employed.

### Results

### Distant-Metastasis-Free Survival Rates in MS Low-Risk and High-Risk Groups

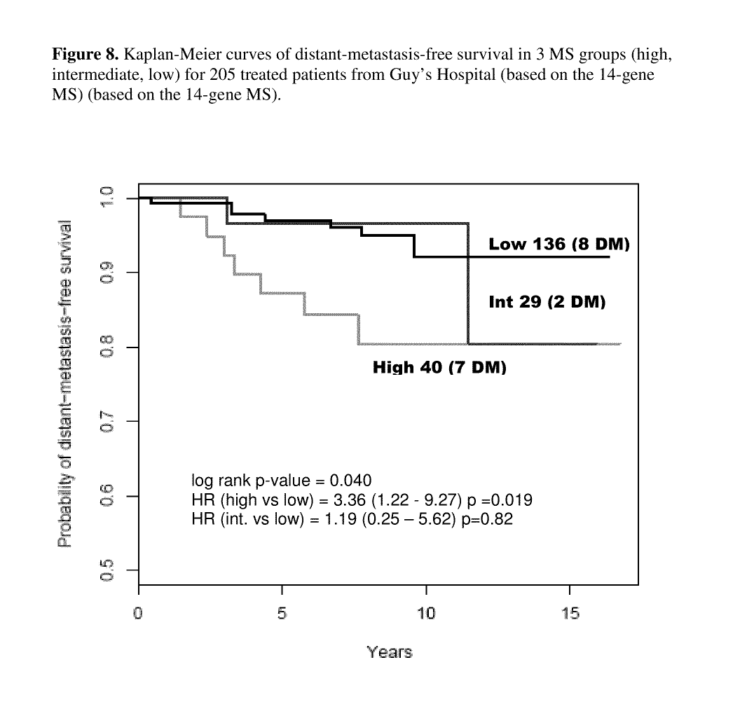

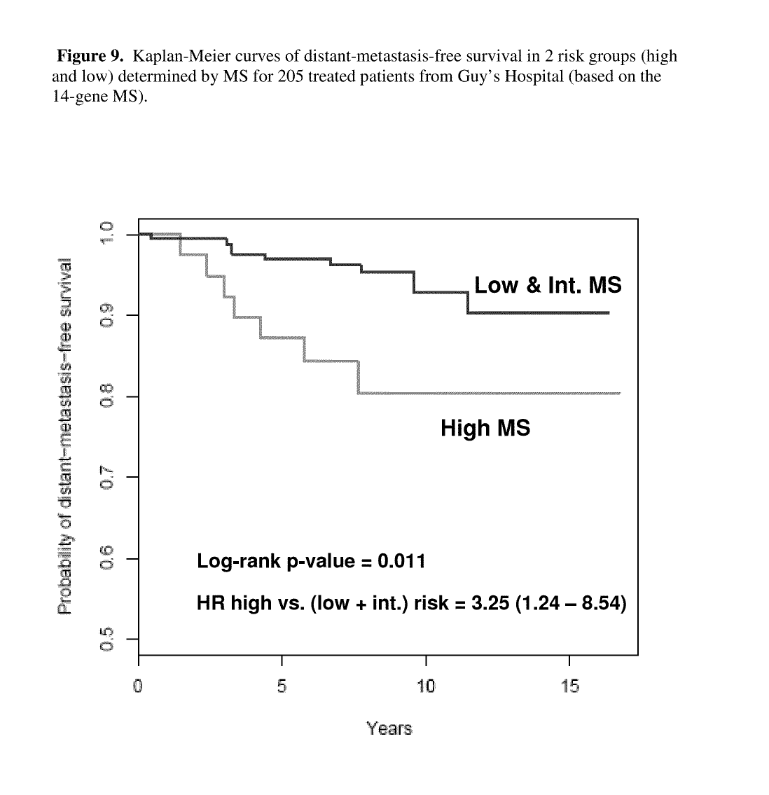

There were 8 distant metastases in the low MS group of 136 individuals, 2 distant metastases in the intermediate MS group of 29 individuals and 7 distant metastases in the high MS group of 40 individuals. The 10-year DMFS rates (SE) were 0.921 (0.028), 0.966 (0.034) and 0.804 (0.068) for low, intermediate, and high MS groups, respectively. There were significant differences in DMFS rates with a log-rank p-value of 0.04. As DMFS rates were similar in low and intermediate MS groups, they were combined to form the low-risk group. The low-risk group had a 10-year DMFS rate of 0.928 (0.025) and was significantly different from the corresponding rate of 0.804 (0.068) for the high-risk group (Table 18). The log-rank p-value was 0.011. Kaplan-Meier plots of distant-metastasis-free survival for the three MS groups and the two MS risk groups were in FIG. 8 and FIG. 9, respectively.

Hazard Ratios from Univariate and Multivariate Cox Regression Models

The unadjusted hazard ratio of the MS high-risk vs. low-risk groups to predict time to distant metastases was 3.25 (95% CI=1.24 to 8.54, p-value=0.017). When adjusted by age, tumor size and histological grade, the hazard ratio was 5.82 (1.71-19.75, p=0.0047) in Table 19. MS risk group was the only risk factor that was significant in the multivariate analyses. Therefore, the gene signature has independent prognostic value for DMFS over the traditional clinicopathological risk factors and captures part of the information of these factors.

Association of MS Risk Groups with Other Clinical and Pathological Characteristics

MS risk group had very significant association with histological grade (Crammer's V=0.65, p<0.0001 for chi-sq test for association). For 60 grade 1 tumors, none (0%) was MS high-risk. In contrary, 9 (9.2%) of 98 grade 2 and 31 (66%) of 47 grade 3 tumors were MS high-risk.

While tumor size was larger in the MS high-risk group, the difference was not statistically different (1.87 cm and 1.61 cm in MS high and low-risk groups respectively, ANOVA p-value=0.14). There was no significant association of MS risk groups with age (Mean age is 59.3 in both high and low-risk groups, ANOVA p=0.34). Results were summarized in Table 20.

### Performance of the Molecular Signature in Different Clinical Subgroups

Hazard ratio of MS risk groups was 3.7 (1.1-12.6) in tumors <2 cm and 3.0 (0.6-14.8) in tumors >2 cm. In women younger than 55 years, HR was 7.4 (1.0-54.7) while it was 2.8 (0.88-8.8) in women older than 55 years. For tumors of histological grade 1 and 2, HR was 12.8 (3.8-43.1) while HR was 1.53 (0.16-14.7) in grade 3 tumors (Table 21).

### Diagnostic Accuracy and Predictive Values

Sensitivity, specificity, PPV and NPV of MS risk groups to predict distant metastasis in 5 years were shown in Table 22. Sensitivity of MS risk group was 0.50 (0.50-0.76) while specificity was 0.82 (0.76-0.87). Using the estimated 5-year distant metastasis rate of 0.05, PPV and NPV were estimated to be 0.13 (0.068-0.23) and 0.97 (0.94-0.98) respectively (Table 22). High NPV of the MS risk group was important for it to be used for ruling out more aggressive treatment such as chemotherapy for patients with low-risk.

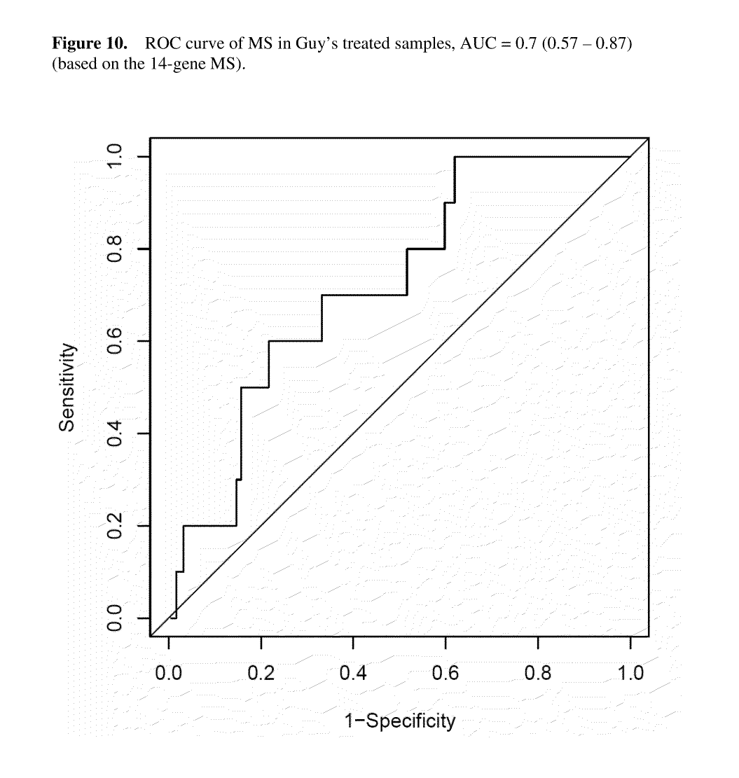

ROC curve of continuous MS to predict distant metastasis within 5 years is shown in FIG. 10 and AUC was estimated to be 0.72 (0.57-0.87).

### Time Dependence of the Prognostic Signature

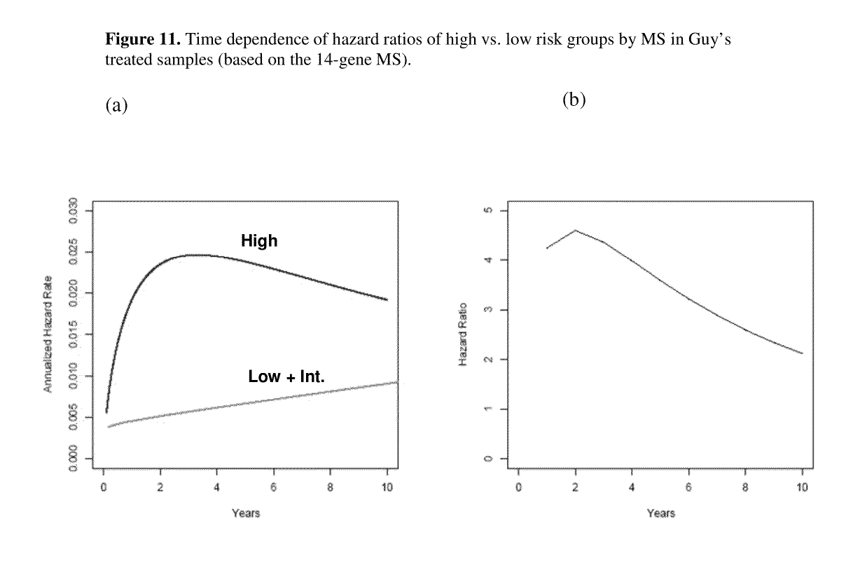

Annualized hazard rates for MS high and low-risk groups were shown in FIG. 11a while the time-dependence of the hazard ratio between two groups was shown in FIG. 11b. For the high-risk group, annual hazard rate peaked at 2.5% around year 3 from surgery and then slowly decreased over the next few years. However, the annualized hazard rate in the low-risk group showed slight but steady increase in the 10-year period that had follow-up. Subsequently, hazard ratio of MS risk groups was time dependent. It was 4.6, 3.6 and 2.1 at year 2, 5 and 10, respectively.

### Example Five

### The 14-Gene Signature Predicts Distant Metastasis in Adjuvant Hormonally-Treated Node-Negative and ER-Positive Breast Cancer Patients Using 234 FFPE Samples

### Patients

A cohort of 234 Japanese women with N−, ER+ breast cancer who had surgery between 1995 and 2003 in Aichi Cancer Center was selected. The median follow-up was 8.7 years. Among them, there were 31 (13%) distant metastases, 19 deaths (8.1%) and 46 (19.7%) local and distant recurrences.

146 (62%) patients were at stage I while 88 (38%) were at stage II. All patients received adjuvant hormonal treatment but no chemotherapy. 112 post-menopausal women were treated with Tamoxifen. Of 122 pre-menopausal women, 102 received Tamoxifen while 20 received Zoladex treatment. The cohort had a mean (SD) age of 53 (11) years and mean (SD) tumor diameter of 2.05 cm (1.1). 74 (32%), 113 (48%) and 47 (20%) patients had tumors of histological grade 1, 2 and 3, respectively. (Table 23)

### Gene Expression Signature and Metastasis Score (MS)

A 14-gene expression signature was previously developed and validated in profiling studies in US and Europe using RT-PCR with FFE samples. Pathway analyses by the program Ingenuity revealed that the majority of the 14 genes in the signature are involved with cell proliferation. Ten of 14 genes are associated with TP53 signaling pathways that have been found to be coordinately over-expressed in tumors of poor-outcome.

A metastasis score (MS) was calculated for each individual. MS was based upon the negative of the mean of the gene expression levels (in AACt) of the 14 genes in the signature. Moreover, two cut points had previously been determined from a study with tumor samples from untreated patients from Guy's Hospital (Example 2) to group patients into high, intermediate and low MS groups.

### Statistical Analyses

Kaplan-Meier (KM) curves for distant metastasis free and overall survival were generated for the high, intermediate and low MS groups. Upon examining the DMFS rates of the three groups, intermediate and low MS groups were combined as a low-risk group which was compared with the high risk group with high MS. Log rank tests were performed.

Univariate and multivariate Cox proportional hazard regression analyses of MS groups for DMFS and OS endpoints were performed. Hazard ratio of high-risk (high MS) vs. low-risk (intermediate and low MS groups combined) group was adjusted for age (in years), tumor size (in cm) and histological grade in one multivariate analysis. In another multivariate analysis, it was adjusted for menopausal status, treatment, tumor size, histological grade and PgR status.

Association of MS groups with age, tumor size was investigated using ANOVA tests while association of MS groups with histological grade and tumor subtypes was evaluated by Crammer's V for strength of associated and by chi-sq tests for statistical significant.

Hazard ratios of the MS risk groups were also calculated for different clinical subgroups. Those groups include pre-menopausal vs. post-menopausal, age ≦55 years vs. >55 years, tumor size >2 cm vs. ≦2 cm, and histological grade 1 & 2 vs. grade 3, PgR+ve vs. −ve.

To assess diagnostic accuracy, Receiver Operator Characteristic (ROC) curve of MS to predict distant metastases within 5 years was plotted. Area under the ROC curves (AUC) was calculated. Sensitivity, specificity, positive predictive value (PPV) and negative predictive value (NPV) were calculated with 95% confidence interval (95% CI) for high vs. low-risk groups by MS.

The time-dependence of hazard ratio of MS groups was investigated by estimating the annualized hazards using a spline-curve fitting technique that can handle censored data. The HEFT procedure in R2.4.1 was employed. Annualized hazards were estimated for both MS high and low-risk groups and from which, the hazard ratios at different time were calculated.

Kaplan Meier estimates and Cox proportional hazard regression were performed using R2.41.1 and SAS 9.1. ROC curves and AUC were estimated using the Mayo Clinic's ROC program. The Delong method of estimating confidence interval of AUC was employed.

### Results

### Distant-Metastasis-Free Survival Rates in MS Low-Risk and High-Risk Groups

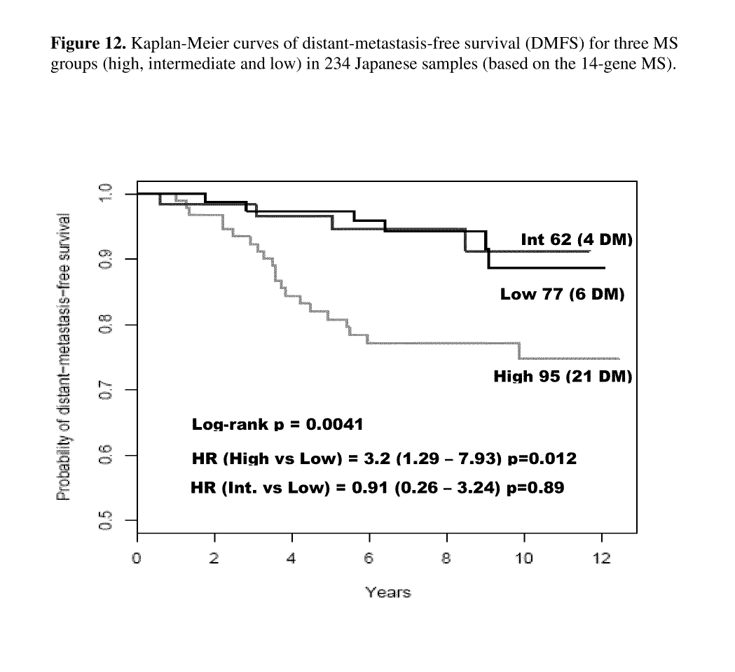

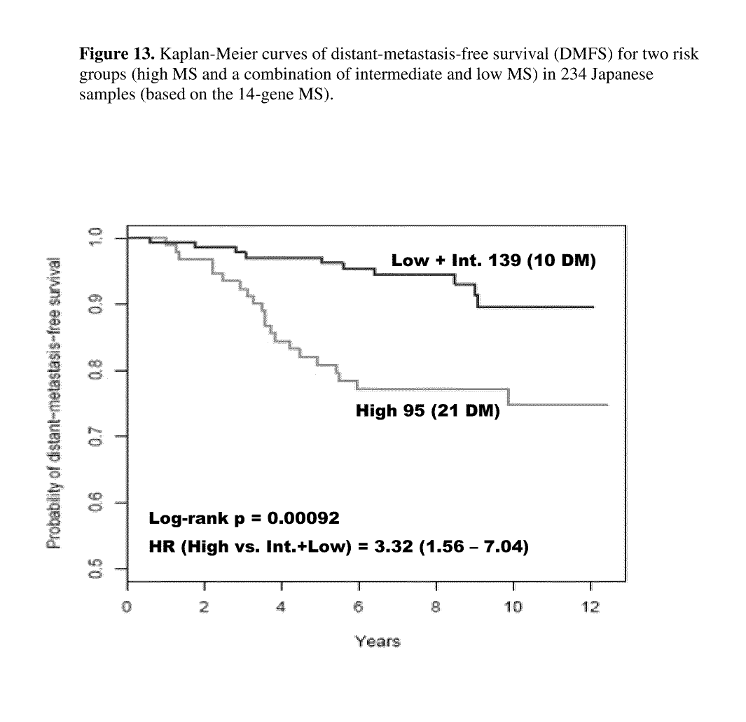

There were 6 distant metastases in the low MS group of 77 individuals, and 4 distant metastases in the intermediate MS group of 66 individuals and 21 distant metastases in the high MS group of 95 individuals. The 10-year DMFS rates (SE) were 0.89 (0.05), 0.91 (0.04) and 0.75 (0.05) for low, intermediate, and high MS groups, respectively. There was significant difference in DMFS rates with a log-rank p-value of 0.004. As DMFS rates were similar in low and intermediate MS groups, they were combined to form the low-risk group. The low-risk group had a 10-year DMFS rate of 0.895 (0.034) and is significantly different from the corresponding rate of 0.75 (0.05) for the high-risk group (Table 24). The log-rank p-value is 0.00092. Kaplan-Meier plots of distant-metastasis-free survival for three MS groups were in FIG. 12 while Kaplan-Meier plots for the two risk groups (high MS and a combination of intermediate and low MS) were in FIG. 13.

Hazard Ratios from Univariate and Multivariate Cox Regression Models

The unadjusted hazard ratio of the MS high-risk vs. low-risk groups to predict time to distant metastases was 3.32 (95% CI=1.56 to 7.06, p-value=0.0018). When adjusted by age, tumor size and histological grade, the hazard ratio was 3.79 (1.42-10.1, p=0.0078). Beside MS, tumor size is the only other significant factor in the multivariate analyses with HR of 1.4 per cm increase (p=0.007) (Table 25). In another multivariate analysis, MS risk groups were adjusted by menopausal status, treatment, tumor size, histological grade and PgR status. The adjusted hazard ratio of MS risk group was 3.44 (1.27-9.34) (Table 27). Again, tumor size was the only other significant factor in this multivariate analysis (HR=1.45 per cm increase, p=0.0049). Therefore, the gene signature has independent prognostic value for DMFS over the traditional clinicopathological risk factors and captures part of the information of these factors.

Association of MS Risk Groups with Other Clinical and Pathological Factors

MS risk group had very significant association with histological grade (Crammer's V=0.54, p <0.0001 for chi-sq test for association). For 74 grade 1 tumors, only 4 (5.4%) were MS high-risk. In contrary, 54 (47.8%) of 113 grade 2 and 37 (78.7%) of 47 grade 3 tumors were MS high-risk.

MS risk groups were also associated with tumor subtypes (Cramer's V=0.25, p=0.02). While 23 (29.8%) of 77 Scirrhous tumors were MS high-risk, 45 (41.7%) of 108 Papillotubular tumors and 24 (63.2%) of 38 solid-tubular were MS high-risk.

While tumor size is larger in the MS high-risk group (2.23 cm and 1.93 in MS high and low-risk groups respectively, ANOVA p-value=0.037), there was no significant association of MS with age (p=0.29). Results were summarized in Table 26.

### Performance of the Molecular Signature in Different Clinical Subgroups

MS risk group can best predict distant metastases in young (age ≦55 years), pre-menopausal women with tumors that were ≦2 cm, low grade (grade 1 and 2) and PgR+ve. Hazard ratio of MS risk groups was 4.5 (1.2-17.3) in tumors ≦2 cm and 2.3 (0.92-5.6) in tumors >2 cm. In pre-menopausal women, HR was 6.0 (1.6-23.3) while it was 2.1 (0.83-5.1) in post-menopausal women. For those with tumors of histological grade 1 and 2, HR was 3.6 (1.5-8.4) while HR was 2.4 (0.29-18.8) in grade 3 tumors. HR of MS risk group was 3.5 (1.4-9.0) in PgR+ve tumors while it was 2.1 (0.57-7.49) in PgR −ve tumors (Table 28).

### Diagnostic Accuracy and Predictive Values

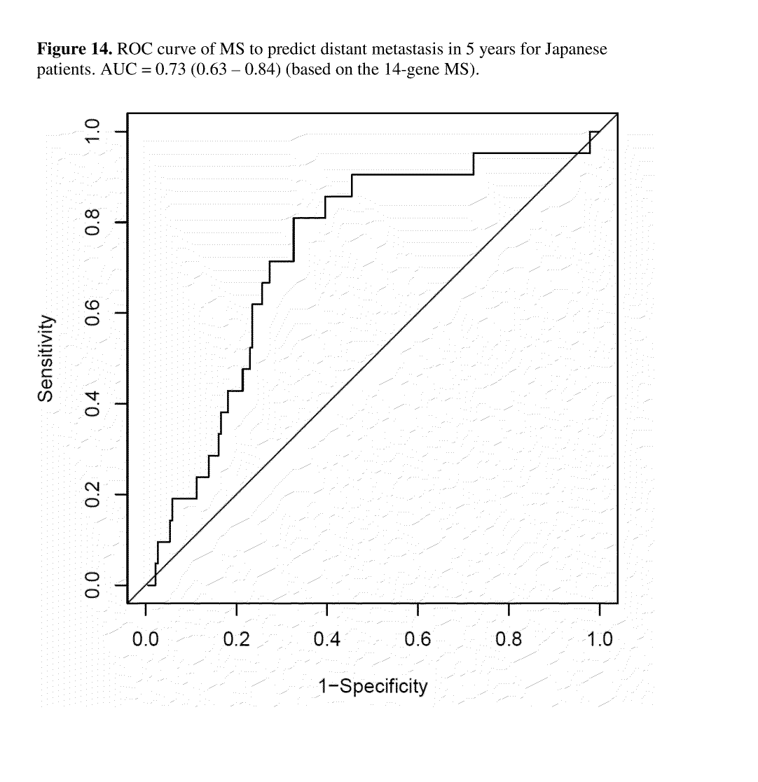

Sensitivity, specificity, PPV and NPV of MS risk groups to predict distant metastasis in 5 years were shown in Table. Sensitivity of MS risk group was 0.81 (0.60-0.92) while the specificity was 0.65 (0.58-0.71). Using the estimated 5-year distant metastasis rate of 0.095, PPV and NPV were estimated to be 0.19 (0.15-0.24) and 0.97 (0.93-0.99) respectively (Table 29). High NPV of the MS risk group was important for it to be used for ruling out more aggressive treatment such as chemotherapy for patients with low-risk. ROC curve of continuous MS to predict distant metastasis within 5 years was shown in FIG. 14 and AUC was estimated to be 0.73 (0.63-0.84).

### Time Dependence of the Prognostic Signature

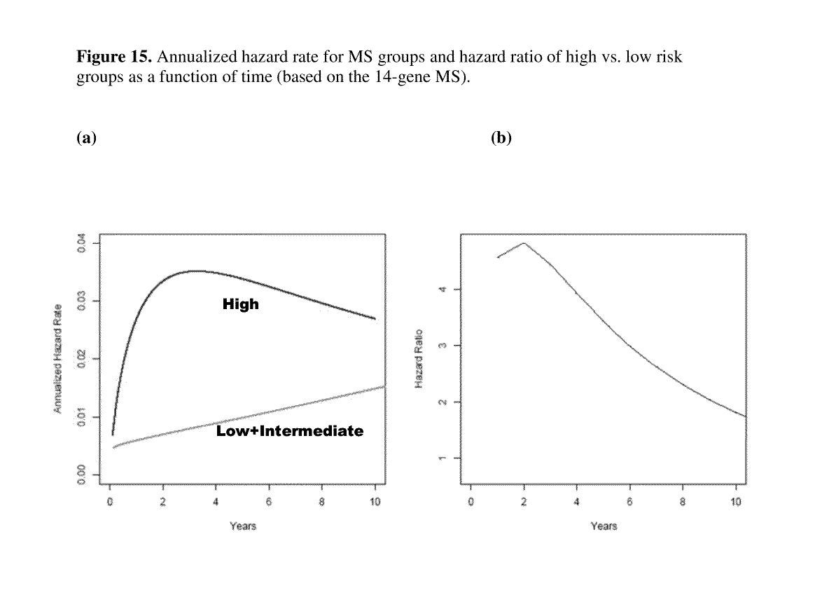

Annualized hazard rates for MS high and low-risk groups were shown in FIG. 15a while the time-dependence of the hazard ratio between two groups was shown in FIG. 15b. For the high-risk group, annual hazard rate peaked at 3.5% around year 3 from surgery and then slowly decreased over the next few years. However, the annualized hazard rate in the low-risk group showed slight but steady increase in the 10-year period that had follow-up. Subsequently, hazard ratio of MS risk groups was time dependent. It was 4.8, 3.4 and 1.8 at year 2, 5 and 10, respectively.

As seen from this example and the previous examples, the 14 gene signature is shown to be an effective risk predictor in breast cancer patients of both Caucasian and Asian ethnic background, indicating the robustness of the 14 gene prognostic signature.

### Example Six

### The 14-Gene Signature, and a Metastasis Score (MS) Derived Therefrom, Predict Response to Chemotherapy

Introduction and Overview

While older postmenopausal women with ER+, HER2− tumors represent the largest fraction of women diagnosed with primary breast cancer, they are less responsive to chemoendocrine therapy than younger women and have been underrepresented in molecular profiling of randomized trials. International Breast Cancer Study Group (IBCSG) Trial IX, which is a randomized controlled trial in postmenopausal women (median age 61 years) with lymph node-negative (N−) disease, showed that patients with ER+ tumors derived no benefit from 3 cycles of cyclophosphamide, methotrexate, 5 fluorouracil (CMF) chemotherapy prior to tamoxifen (T) treatment, whereas patients with ER-tumors did derive substantial benefit (IBCGS, J Natl Cancer Inst. 2002, 94:1054 and Aebi et al., Ann Oncol. 2011; 9; 1981).

A 14-gene expression signature, referred to as a metastasis score (“MS”), that is prognostic of breast cancer distant metastasis in women with ER+ and node-negative breast cancer has been described (Tutt et al., BMC Cancer. 2008; 8:339, which is incorporated herein by reference in its entirety, as well as Examples One through Five above and elsewhere herein). The MS, as previously described, was derived from and validated in untreated women.

Herein in Example Six, it was analyzed whether MS (the 14-gene MS provided herein in exemplary embodiments) could identify a subset of postmenopausal women who differentially benefit from addition of chemotherapy to tamoxifen (T) treatment, based on Trial IX (the analysis was limited to patients with ER (+), HER2 (−) breast cancer because patients with HER2 (+) tumors would typically be treated with Herceptin® or other HER2 receptor-binding drugs).

Accordingly, following ER stratification, 1669 eligible patients (1040 with ER+, HER2-tumors) were randomized to receive 3 courses of CMF→T vs. T alone. RNA was available from 864 patients, of which 803 were successfully profiled by RT-PCR. The expression level of 14 equally-weighed proliferation genes and 3 normalization genes were used to generate MS. The analysis was limited to the first seven years of follow-up (which is the mean follow-up time for Trial IX) of 568 ER+, HER2− patients for disease-free survival (DFS) and breast cancer-free interval (BCFI), as well as overall survival (OS) and disease recurrence-free interval (DRFI). Patient characteristics of this subgroup of 568 patients were similar to samples in the entire trial and patients in the two treatment arms were balanced (Tables 31-32).

For further information regarding IBCSG Trial IX, see International Breast Cancer Study Group (IBCSG), “Endocrine responsiveness and tailoring adjuvant therapy for postmenopausal lymph node-negative breast cancer: a randomized trial”, J Natl Cancer Inst. 2002 Jul. 17; 94(14):1054-65, incorporated herein by reference in its entirety.

The benefit of CMF→T over T was statistically significant in the patients with low MS. The 7 yr DFS estimate was 94.5% (89.9%-99.3%) for MS low patients and 80.9% (75.4%-86.8%) for MS high patients, representing a 3.94 fold increase in the relative hazard of disease for MS High over MS Low in CMF→T treated patients. Similarly, the survival estimate for BCFI was 95.6% (91.4%-99.9%) for MS low and 84.2% (79.0%-89.6%) for high MS, representing a 3.95 fold increase in the relative hazard of breast cancer recurrence for MS high over MS low in CMF→T treated patients. The effect of MS was negligible and constant over time among women in the T-only arm whereas, in contrast, increased MS in the CMF-T arm was associated with worse DFS during the first seven years of follow-up.

Thus, postmenopausal breast cancer patients with ER+, HER2− tumors with low MS benefitted substantially from 3 cycles of CMF whereas patients with high MS obtained no benefit.

Specifically, low MS was associated with differential benefit favoring those women receiving CMF→T vs T alone for both DFS and BCFI. The effect was independent of traditional risk factors including Ki-67. Postmenopausal patients with high MS did not benefit from 3 cycles of CMF. Thus, a high MS may identify those patients who would be candidates for alternative treatments such as targeted therapies.

Thus, the analysis herein in Example Six, which was unconfounded by chemotherapy-induced ovarian ablation in younger women, demonstrates that MS can be used to identify a subset of postmenopausal women with ER+, HER2− tumors who benefit from chemotherapy, such as CMF chemotherapy. In particular, ER+, HER2− patients with low MS tumors benefit from the addition of chemotherapy (e.g., CMF) whereas those with high MS tumors do not.

Methods

Patients

Trial IX evaluated the role of adjuvant chemotherapy preceding tamoxifen treatment in postmenopausal patients with lymph node-negative breast cancer. Postmenopausal status was defined as a) older than 52 years with at least 1 year amenorrhea; b) 52 years or younger with at least 3 years amenorrhea, c) 56 years old or older with hysterectomy but no bilateral oophorectormy; or d) biochemical evidence of cessation of ovarian function (International Breast Cancer Study Group (IBCSG). “Endocrine responsiveness and tailoring adjuvant therapy for postmenopausal lymph node-negative breast cancer: a randomized trial”, J Natl Cancer Inst. 2002; 94(14):1054-65. Erratum in: J Natl Cancer Inst 2002; 94(17):1339). From Oct. 1, 1988, through Aug. 1, 1999, a total of 1669 eligible and assessable patients were stratified by estrogen receptor (ER) status and randomly assigned to receive three 28-day courses of CMF (cyclophosphamide at 100 mg/m² on days 1-14, orally; methotrexate at 40 mg/m² on days 1 and 8, intravenously; and 5-fluorouracil at 600 gm/m² on days 1 and 8, intravenously) followed by tamoxifen (20 mg/day, orally for 57 months) (CMF→T) or to receive tamoxifen (T) alone (20 mg/day, orally for 60 months). Institutional review boards reviewed and approved the protocols, and informed consent was acquired according to the criteria established within the individual countries that participated in this international trial.

Sample Preparation

RNA from FFPE sections was extracted at The Consortium of Clinical Diagnostics. FFPE sections were deparaffinized with xylene, washed twice with 100% ethanol and dried. The tissues were digested in RML buffer containing proteinase K at 50° C. on a shaking incubator (900 rpm). Following overnight digestion, the samples were incubated at 80° C. for 15 minutes and spun down. The RNA was isolated using the Omega's Mag-Bind FFPE RNA 96 KF on the Thermo Scientific Kingfisher Flex. Of the 1669 patients assessed in Trial IX, RNA was available from 864 patients for molecular profiling.

Gene Expression Profiling

The breast cancer assay first described by Tutt et al. (“Risk estimation of distant metastasis in node-negative, estrogen receptor-positive breast cancer patients using an RT-PCR based prognostic expression signature”, BMC Cancer. 2008; 8:339) consisted of singleplex amplifications of 14 proliferation-associated genes plus three normalization genes. The PCR products, which ranged from 100-200 bp, were detected using SYBR green intercalation; specificity was determined by melt profiles. To improve RT-PCR amplification of archival FFPE samples with low RNA content and/or significant sample degradation, primers were re-designed to target shorter RNA fragments and the assays were reconfigured into 5 multiplex TaqMan assays (as shown in Table 30). It was subsequently shown that the results from the two assay formats correlated well. The 5 multiplex TaqMan assays were used to analyze the 14 genes in samples from Trial IX.

Specifically, the RNAs were reversed transcribed using the High Capacity cDNA kit (Applied Biosystems) and gene-specific primers for the 14 constituent genes of MS, 3 normalization genes (NUP214, PPIG, and SLUT), plus ESR1, PGR and ERBB2. The mRNA expression levels were quantified using 5 multiplex RT-PCR TaqMan® assays. The composition of genes in each multiplex and the amplicon lengths are shown in Table 30. With the exception of the 3 normalization genes in pool 5, each gene in the multiplex was detected with a spectrally unique fluorophore. Probes labeled with FAM, VIC, or NED and conjugated with minor groove binder groups were purchased from Applied Biosystems. The Quasar 670-labeled probes containing BHQ quenchers were purchased from Biosearch Technologies. The assays were performed in duplicate on the Prism 7500 Real-Time PCR system for 45 cycles using TaqGold DNA polymerase. A Universal Human Reference RNA (Stratagene), reversed transcribed and amplified along with the samples in every run, was used to normalize run-to-run differences.

A metastasis score (MS) was generated from the normalized expression level of 14 prognostic genes as previously described (Tutt et al., BMC Cancer. 2008; 8:339). ESR1, PGR, and ERBB2 RT-PCR results, which can be used to establish hormonal and HER2 status, were not used in generating the MS (but each could optional be used in conjunction with the MS). The MS was evaluated both categorically (using a cutpoint) and on a continuous scale. The unscaled MS (MSu) was determined by summing the Δ(ΔCt) for each of the 14 genes and rescaled to positive using the formula (MSu+60)/2. The −23.5 cutpoint used in the original singleplex assay described by Tutt et al. was similarly rescaled to positive to 18.25, or (−23.5+60)/2. For the multiplex TaqMan assay described here, the cutpoint (for categorical analysis using the MS) was further adjusted to 17.5 to account for slight differences between the assay formats.

Study Endpoints

Endpoints of interest were disease-free survival (DFS), overall survival (OS), and breast cancer-free interval (BCFI) and disease recurrence-free interval (DRFI). DFS was defined as the length of time from the date of randomization to any invasive breast cancer relapse (including ipsilateral or contralateral breast recurrence), the appearance of a second nonbreast malignancy, or death, whichever occurred first. OS was defined as the length of time from the date of randomization to death from any cause. BCFI was defined as the length of time from the date of randomization to any invasive breast cancer relapse (including ipsilateral or contralateral breast recurrence) and DRFI was defined as the length of time from the date of randomization to breast cancer recurrence but ignoring local, regional and contralateral breast events.

Statistical Analyses

Comparisons of baseline categorical measures for samples where RNA was available and profiled for MS compared to samples that were not profiled for MS were assessed by chi squared tests, those of an ordinal nature were assessed by Mantel-Haenszel correlation tests, and continuous measures were assessed by Wilcoxon tests. Data for continuous variables not profiled for MS were not available for comparison. Analysis was limited to the first 7 years of follow-up.

Differences between the survival experiences of ER+/HER2− patients with high MS vs. those with low MS in each treatment arm were assessed by plots of the Kaplan-Meier product-limit estimates (Collett, “Modelling Survival Data in Medical Research”, 2nd Ed. (2003)). Standard errors for the Kaplan-Meier estimates were calculated by the Greenwood's formula (Collett, “Modelling Survival Data in Medical Research”, 2nd Ed. (2003)). The predictive value of the MS on the endpoints for ER+/HER2− patients treated with either Tamoxifen alone or CMF-Tamoxifen was assessed by Cox proportional hazards (PH) models (Collett, “Modelling Survival Data in Medical Research”, 2nd Ed. (2003)). For categorical analyses of MS (or Ki-67), values ≧17.5 (or ≧12 for Ki-67) were dichotomized as “high”. Tied survival times were handled by the method of Efron (Efron, B. (1977), “The efficiency of Cox's likelihood function for censored data”, Journal of the American Statistical Association, 72, 557-565). Treatment-MS (or Ki-67) interaction effects were adjusted for age of subject at randomization, local treatment (surgical/radiologic intervention), tumor size, and tumor grade. Interaction effects were assessed by nested likelihood ratio tests (LRT) of covariate adjusted models fit with and without a treatment-MS interaction term.

Plots of Cox PH scaled Schoenfeld residuals (Therneau et al., “Modeling Survival Data: Extending the Cox Model” (2000)) vs. time were generated to visualize changes in the parameters for MS, treatment, and MS*treatment interaction effects over time.

Subpopulation Treatment Effect Pattern Plots (STEPP) (Bonetti et al., “A graphical method to assess treatment-covariate interactions using the Cox model on subsets of data”, Stat Med 2000; 19:2595-609) were generated to study the treatment-MS (or Ki-67) interaction effects. Relative changes in Cox PH hazard ratios and 5-year survival estimates for each treatment were visualized across levels of MS and Ki-67.

To evaluate the possible influence of non-cancer related deaths on overall survival, an analysis of overall survival was performed where deaths without a recurrent tumor were censored at the time of death.

Results

Of the 1669 patients assessed in Trial IX, RNA was available from 864 patients for molecular profiling. Of these, 803 were successfully profiled by RT-PCR and stratified by ER and HER2 IHC status. This analysis was limited to 568 ER+/HER2− samples, of which 291 (51%) and 277 (49%) were from the T and CMF→T arms, respectively. The clinical characteristics of the 568 patients are similar to the parent trial with respect to age, tumor grade, tumor size, local treatment and hormonal status (Table 31); samples in the two treatment arms are balanced (Table 32).

Of the 568 samples, 169 samples (30%) were classified as low risk by MS and 399 samples (70%) were classified as high risk. Kaplan-Meier curves estimating DFS, for patients classified as low and high MS over the 19 year follow-up period were plotted. Visual inspection of the curves suggested the effect of MS on DFS changes over time. This was investigated further by modeling MS as a time-dependent variable in a Cox model and plotting the estimated effect of MS on DFS over 19 years of follow-up separately by treatment arm. These plots suggested that the effect of MS was negligible and constant over time among women in the Tam-only arm whereas, in contrast, the effect of MS in the CMF→T arm depends on time. Specifically, among CMF→T treated patients, increasing MS was associated with worsening DFS during the first 7 years of follow-up followed by a period of decreased risk during the follow-up period subsequent to 7 years. Therefore, further analysis was limited to the first 7 years of follow-up.

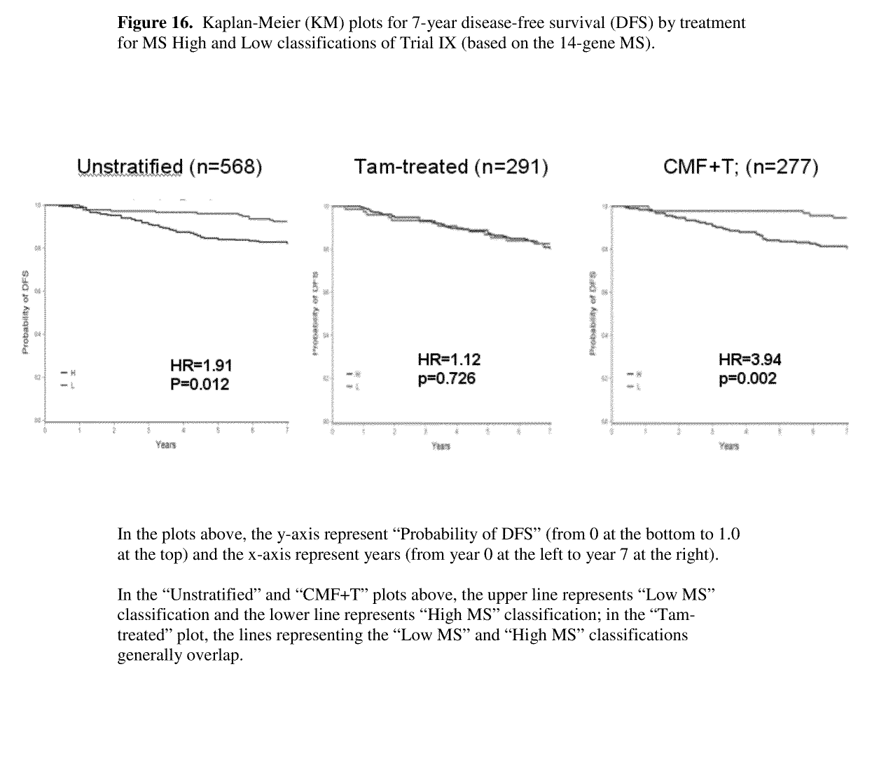

Seven year estimates of DFS, BCFI, OS and DRFI are presented in Table 33 and Kaplan-Meier curves for DFS for patients classified as MS High and MS Low in FIG. 16. For individuals treated with CMF→T, the Tyr DFS estimate for MS low patients was 94.5% (89.9%-99.3%), for MS high patients the estimate was 80.9% (75.4%-86.8%), representing a 3.94-fold increase in the relative hazard of disease for MS High over MS Low in CMF→T-treated patients. Similarly, the survival estimate for BCFI of those CMF→T patients with MS low patients was 95.6% (91.4%-99.9%) and 84.2% (79.0%-89.6%) for high MS, representing a 3.95-fold increase in the relative hazard of breast cancer recurrence for MS high over MS low in CMF→T treated patients. Estimates of Tyr OS and DRFI did not differ significantly for low or high MS patients in either treatment arm. The relative differences in Kaplan-Meier estimates of DFS and BCFI at 5 years are similar to those at 7 years (Table 36 demonstrates the predictive association of MS at 5 years).

Analysis of MS in multivariate adjusted Cox models suggest both the continuous and categorical scores are predictive of DFS and BCFI and remain significant when adjusted for Ki-67 in the model (Table 34 and Table 35). Tests of MS by treatment interactions in all models suggest that the relative hazards for DFS and BCFI for CMF→T vs. T-treated individuals were different among patients according to MS (MS-treatment interaction p<0.004 and p<0.01 for categorical and continuous models of MS respectively). Patients categorized as having low MS had improved DFS and BCFI in the CMF→T arm relative to the T arm but there was no improvement for patients with high MS. Similarly, models of continuous MS estimate improved DFS and BCFI in the CMF→T arm relative to the T arm for a subject with MS at the 25th percentile (MS=16.5) but no improvement for those with MS at the 75th percentile (MS=24.0). OS and DRFI endpoints showed trends similar to those for the DFS and BCFI endpoints but reached statistical significance only for models of continuous MS.

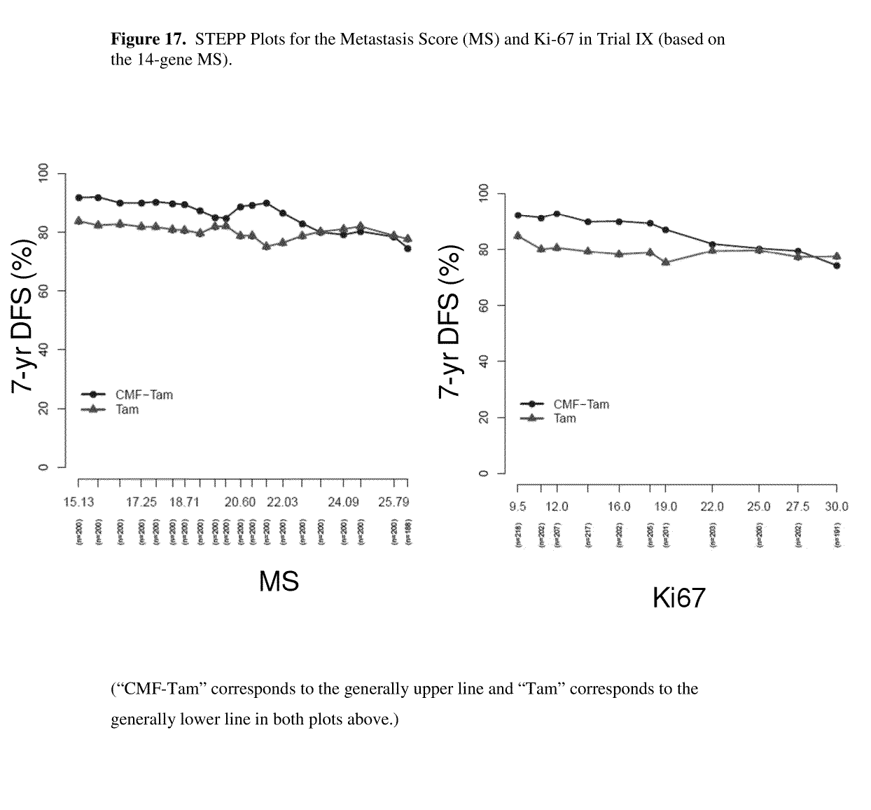

STEPP analyses for subpopulations of MS and Ki-67 suggest that 7 year DFS is greater for patients in the CMF→T arm relative to the T only arm when MS is low, but the protective effect of CMF→T decreases relatively faster than for T alone and inverts for high values of MS or Ki-67 (FIG. 17). MS correlated modestly with log % Ki-67 (r=0.47) but not with % ER or % PR.

Cox PH analysis of overall survival, where deaths without recurrence were censored at time of death, confirm the overall survival observation that the relative hazard of death in the CMF→T arm is greater than and different from that of those in the T-alone arm for increasing MS.

Discussion

Although Trial IX failed to demonstrate the benefit of preceding tamoxifen by 3 cycles of CMF for ER+, N− tumors (IBCGS, J Natl Cancer Inst. 2002, 94:1054 and Aebi et al., Ann Oncol. 2011; 9; 1981), herein in Example Six it was analyzed whether a previously described proliferation score, MS, could identify a subset of women who differentially benefit from addition of chemotherapy to tamoxifen in Trial IX. In the analysis carried out herein in Example Six, a subset of postmenopausal women ≧50y with ER+/HER2− breast cancer was identified who benefit from CMF chemotherapy. It was determined that, unexpectedly, women with tumors with low MS did benefit from the addition of 3 cycles of CMF to tamoxifen while those with high MS tumors did not benefit. The results of the analysis herein in Example Six contrast with results from NSABP20 (Fisher et al., “Tamoxifen and chemotherapy for lymph node-negative, estrogen receptor-positive breast cancer”, J Natl Cancer Inst. 1997 89(22):1673-82), the only other trial that compares CMF plus tamoxifen vs. tamoxifen alone, in that patients with low risk scores as determined by a multi-gene expression signature (low MS scores in this example) actually benefitted more from chemotherapy. In the NSABP20 study, patients with a low recurrence score (RS), as determined by Oncotype Dx®, did not benefit from the addition of chemotherapy while those with high RS derived significant benefit (Paik et al., “Gene expression and benefit of chemotherapy in women with node-negative, estrogen receptor-positive breast cancer”, J Clin Oncol. 2006 Aug. 10; 24(23):3726-34).

A major difference between Trial IX and NSABP20 is the age and menopausal status of the patients enrolled. While 55% of the patients in NSABP20 were >50 yrs old, 98% were >50 yrs old in Trial IX, of whom 55% were >60 yrs old. All patients in Trial IX were postmenopausal vs. 53% in NSABP20 (Fisher et al., “Tamoxifen and chemotherapy for lymph node-negative, estrogen receptor-positive breast cancer”, J Natl Cancer Inst. 1997 89(22):1673-82).

This indicates that, especially in postmenopausal women, a low MS score (e.g., an MS score that is below a certain cutoff/threshold value) indicates an increased likelihood of responding to (benefitting from) chemotherapy, whereas a high MS score (e.g., an MS score that is above a certain cutoff/threshold value) indicates a decreased likelihood of responding to (benefitting from) chemotherapy.

Although anthracycline-based polychemotherapy are among the most effective for the treatment of patients with breast cancer, increased rates of cardiotoxicity, including congestive heart failure, have been observed, particularly in patients older than 65 years. Indeed, reports indicate under-utilization of CAF for this reason. Given the higher incidence of breast cancer in this age group and the continuing growth of this segment of the population, identifying an effective but less toxic treatment regimen is highly desirable. The three courses of CMF used in Trial IX were well tolerated and detrimental effects on quality of life were transient. The results of the analysis herein in Example Six demonstrate that a subset of postmenopausal women with lymph node negative breast cancer whose tumors have a low MS may benefit from this less intensive and less cardiotoxic course of chemotherapy. Patients with high MS tumors in this analysis did not benefit from the three cycles of CMF. Thus, a high MS could identify patients who would be candidates for alternative treatments such as targeted therapeutic agents.

In conclusion, in the analysis presented herein in Example Six, low MS was associated with differential benefit favoring those women receiving CMF chemotherapy in addition to tamoxifen, compared with tamoxifen alone, for both DFS and BCFI in the first seven years. Thus, low MS is predictive of response to chemotherapy (i.e., benefit from chemotherapy). The effect was independent of traditional risk factors including Ki-67. Hence this analysis, which is unconfounded by chemotherapy-induced ovarian ablation in younger women, demonstrates that MS can be used to identify a subset of postmenopausal women with ER+, HER2− tumors that benefit from chemotherapy such as CMF chemotherapy. Specifically, ER+/HER2− breast cancer patients who are identified as having low MS tumors would be identified as benefitting from the addition of chemotherapy such as CMF (e.g., they would be predicted to benefit from the addition of chemotherapy to tamoxifen treatment) whereas those patients identified as having high MS tumors would be identified as not benefitting from chemotherapy (e.g., they would be predicted to not benefit from the addition of chemotherapy to tamoxifen treatment).

### Example Seven

### Subsets of the 14-Gene Signature, and Metastasis Scores (MS) Derived Therefrom, for Predicting Response to Chemotherapy or Risk for Metastasis

The correlation between the full set of 14 genes and all possible subsets of the 14 genes was determined among 803 subjects in the IBCSG Trial IX study (which is described above in Example Six) for whom a metastasis score (MS) derived from the full set of 14 genes was available. There are 16,382 possible subsets of the 14 genes. Patient scores for each subset were calculated as the sum of the Δ(ΔCt) values among the genes included in the subset. Correlation between the MS for the full set of 14 genes and each subset score was assessed using Spearman's correlation coefficient.

Spearman's correlation coefficient is a non-parametric measure of correlation that depends only on the ranked values of each of the variables and thus is not affected if a monotonic transformation is applied to either or both of the two variables. Thus, the Spearman coefficient has the advantageous property that it remains the same if one or both of the scores (i.e., for a subset of 14 genes or the full set of 14 genes) undergoes a simple transformation (such as if the MS is re-scaled from, for example, a sum of Δ(ΔCt) values to a score ranging from 0-40 or 0-60, for example).

Table 40 provides correlation coefficients between an MS that is derived from all possible subsets of the 14 genes with an MS that is derived from the full set of 14 genes described herein, based on data from IBCSG Trial IX. These correlation coefficients indicate how closely results (e.g., a MS) derived from gene expression analysis of each subset of the 14 genes would correlate with results derived from gene expression analysis of the full set of 14 genes, such as for predicting response to chemotherapy or determining risk for breast tumor metastasis. For example, all subsets containing nine or more genes had a Spearman correlation coefficient with the full set of 14 genes of >0.95.

Table 40 provides the Spearman correlation coefficient, as well as the Pearson and Kendall's Tau correlation coefficients, for each subset of genes. The Spearman, Pearson, and Kendall's Tau correlation coefficients are described in, for example, Myles Hollander & Douglas A. Wolfe (1973), “Nonparametric Statistical Methods”, New York: John Wiley & Sons, pages 185-194, which is incorporated herein by reference.

When utilizing a subset of the 14 genes, such as any of the subsets provided in Table 40, if cut-points are utilized for categorizing a sample, they can be adjusted as desired (for example, to categorize a sample as either “low” MS or “high” MS, such as to predict increased chemotherapy response and/or decreased metastasis risk based on a low MS or to predict decreased chemotherapy response and/or increased metastasis risk based on a high MS).

### Example Eight

### Analysis of a 12-Gene Signature, and a Metastasis Score Derived Therefrom, for Predicting Response to Chemotherapy or Risk for Metastasis

Herein in Example Eight, it was analyzed whether a breast cancer assay and MS comprising fewer than all 14 genes could predict response to chemotherapy while requiring the detection and analysis of fewer genes compared with the 14-gene signature (referred to herein as “MS 14”). Accordingly, a gene signature (referred to herein as “MS 12”) was developed in which MYBL2 and CCNB 1 were excluded while retaining the other 12 genes from the MS 14.

Two sample sets from Guy's Hospital were used for initial “discovery” and “validation” sample sets to establish the rescaling factor and cutpoint for this MS 12 gene signature. Excellent correlation was observed between the un-scaled MS12 (“MSu12”) and un-scaled MS14 (“MSu14”) in both of the sample sets from Guy's (Tables 37-38). As with MSu14, the rescaling factor for MSu12 was determined based on the range of MS; the mean score was used as the cutpoint. In this sample set, the MSu12 ranged from −52 to 22.5. The score was rescaled to positive using the formula (MSu12+51.5)/2. The un-scaled mean MS (−20) was adjusted to −21.5 to adjust for slight differences between the SYBR and TaqMan assays. The cutpoint was determined to be 15 using the same formula (−21.5+51.5)/2). Thus, in certain exemplary embodiments, a cutpoint of 15 can be utilized, particularly in embodiments which utilize the 12 genes CENPA, PKMYT1, MELK, BUB1, RACGAP1, TK1, UBE2S, DC13, RFC4, PRR11, DIAPH3, and ORC6L such as to derive a score such as the “MS 12” in this example.

As shown for the discovery sample set in Table 37, 201 of the 206 subjects (97.6%) were categorized the same with respect to having either a low or high MS (low MS being indicative of low risk for metastasis as well as benefit/responsiveness to chemotherapy, and high MS being indicative of high risk for metastasis as well as no benefit/responsiveness to chemotherapy) whether the MSu12 or the MSu14 was utilized (120 subjects were categorized as low-risk by both the MSu12 and the MSu14, and 81 subjects were categorized as high-risk by both the MSu12 and the MSu14), and 5 of the 206 subjects (2.4%) had discordant classification between the MSu12 and MS14 (Table 37). As shown for the validation sample set in Table 38, 280 of the 287 subjects (97.6%) were categorized the same with respect to having either a low or high MS (low MS being indicative of low risk for metastasis as well as benefit/responsiveness to chemotherapy, and high MS being indicative of high risk for metastasis as well as no benefit/responsiveness to chemotherapy) whether the MSu12 or the MSu14 was utilized (100 subjects were categorized as low-risk by both the MSu12 and the MSu14, and 180 subjects were categorized as high-risk by both the MSu12 and the MSu14), and 7 of the 287 subjects (2.4%) had discordant classification between the MSu12 and MS14 (Table 38).

Using a rescaling factor of 51.5 and a cutpoint of 15, the MS12 was determined for 803 Trial IX samples for which MS14 data was available. The risk categorizations for the MS12 and MS14 correlated very well (Table 39). For the Trial IX sample set, 783 of the 803 subjects (97.5%) were categorized the same with respect to having either a low or high MS (low MS being indicative of low risk for metastasis as well as benefit/responsiveness to chemotherapy, and high MS being indicative of high risk for metastasis as well as no benefit/responsiveness to chemotherapy) whether the MS12 or the MS14 was utilized (as shown in Table 39, 195 subjects were categorized as low-risk by both the MS12 and the MS14, and 588 subjects were categorized as high-risk by both the MS12 and the MS14) and 20 of the 803 subjects (2.5%) had discordant classification between the MS14 and MS12 (Table 39). All the discordant samples were close to the cutpoint.

Since outcome data was available for Trial IX, Kaplan Meier curves were compared on a subset of ER(+)/HER2(−) samples using the results obtained with MS14 and MS12. As evident from FIG. 18, the MS14 and MS12 signatures gave very similar results. For subjects with low MS14, there was a 3.5-fold greater hazard of worse disease-free survival (DFS) in Tamoxifen only-treated subjects than in subjects treated with CMF chemotherapy+Tamoxifen (p=0.011). Similarly, for subjects with low MS12, there was a 3.7-fold greater hazard of worse DFS in Tamoxifen only-treated subjects than in subjects treated with CMF chemotherapy+Tamoxifen (p=0.007) (FIG. 18). Thus, this demonstrates that subjects who are categorized as having a low MS (based on either the MS12 or MS14) benefit from chemotherapy (such as CMF or other chemotherapy), such as by adding chemotherapy to tamoxifen treatment.

Thus, the 12-gene signature (the 14-gene signature minus MYBL2 and CCNB1; i.e., the 12 genes CENPA, PKMYT1, MELK, BUB1, RACGAP1, TK1, UBE2S, DC13, RFC4, PRR11, DIAPH3, and ORC6L), and a metastasis score derived therefrom, predicts response to breast cancer chemotherapy as well as risk for breast tumor metastasis, while requiring the detection and analysis of fewer genes compared with the 14-gene signature.

All publications and patents cited in this specification are herein incorporated by reference in their entirety. Various modifications and variations of the described compositions, methods and systems of the invention will be apparent to those skilled in the art without departing from the scope and spirit of the invention. Although the invention has been described in connection with specific preferred embodiments and certain working examples, it should be understood that the invention as claimed should not be unduly limited to such specific embodiments. Indeed, various modifications of the above-described modes for carrying out the invention that are obvious to those skilled in the field of molecular biology, genetics and related fields are intended to be within the scope of the following claims.

Lengthy table referenced here
US20140315844A1-20141023-T00001
Please refer to the end of the specification for access instructions.

Lengthy table referenced here
US20140315844A1-20141023-T00002
Please refer to the end of the specification for access instructions.

Lengthy table referenced here
US20140315844A1-20141023-T00003
Please refer to the end of the specification for access instructions.

Lengthy table referenced here
US20140315844A1-20141023-T00004
Please refer to the end of the specification for access instructions.

Lengthy table referenced here
US20140315844A1-20141023-T00005
Please refer to the end of the specification for access instructions.

Lengthy table referenced here
US20140315844A1-20141023-T00006
Please refer to the end of the specification for access instructions.

Lengthy table referenced here
US20140315844A1-20141023-T00007
Please refer to the end of the specification for access instructions.

Lengthy table referenced here
US20140315844A1-20141023-T00008
Please refer to the end of the specification for access instructions.

Lengthy table referenced here
US20140315844A1-20141023-T00009
Please refer to the end of the specification for access instructions.

Lengthy table referenced here
US20140315844A1-20141023-T00010
Please refer to the end of the specification for access instructions.

Lengthy table referenced here
US20140315844A1-20141023-T00011
Please refer to the end of the specification for access instructions.

Lengthy table referenced here
US20140315844A1-20141023-T00012
Please refer to the end of the specification for access instructions.

Lengthy table referenced here
US20140315844A1-20141023-T00013
Please refer to the end of the specification for access instructions.

Lengthy table referenced here
US20140315844A1-20141023-T00014
Please refer to the end of the specification for access instructions.

Lengthy table referenced here
US20140315844A1-20141023-T00015
Please refer to the end of the specification for access instructions.

Lengthy table referenced here
US20140315844A1-20141023-T00016
Please refer to the end of the specification for access instructions.

Lengthy table referenced here
US20140315844A1-20141023-T00017
Please refer to the end of the specification for access instructions.

Lengthy table referenced here
US20140315844A1-20141023-T00018
Please refer to the end of the specification for access instructions.

Lengthy table referenced here
US20140315844A1-20141023-T00019
Please refer to the end of the specification for access instructions.

Lengthy table referenced here
US20140315844A1-20141023-T00020
Please refer to the end of the specification for access instructions.

Lengthy table referenced here
US20140315844A1-20141023-T00021
Please refer to the end of the specification for access instructions.

Lengthy table referenced here
US20140315844A1-20141023-T00022
Please refer to the end of the specification for access instructions.

Lengthy table referenced here
US20140315844A1-20141023-T00023
Please refer to the end of the specification for access instructions.

Lengthy table referenced here
US20140315844A1-20141023-T00024
Please refer to the end of the specification for access instructions.

Lengthy table referenced here
US20140315844A1-20141023-T00025
Please refer to the end of the specification for access instructions.

Lengthy table referenced here
US20140315844A1-20141023-T00026
Please refer to the end of the specification for access instructions.

Lengthy table referenced here
US20140315844A1-20141023-T00027
Please refer to the end of the specification for access instructions.

Lengthy table referenced here
US20140315844A1-20141023-T00028
Please refer to the end of the specification for access instructions.

Lengthy table referenced here
US20140315844A1-20141023-T00029
Please refer to the end of the specification for access instructions.

Lengthy table referenced here
US20140315844A1-20141023-T00030
Please refer to the end of the specification for access instructions.

Lengthy table referenced here
US20140315844A1-20141023-T00031
Please refer to the end of the specification for access instructions.

Lengthy table referenced here
US20140315844A1-20141023-T00032
Please refer to the end of the specification for access instructions.

Lengthy table referenced here
US20140315844A1-20141023-T00033
Please refer to the end of the specification for access instructions.

Lengthy table referenced here
US20140315844A1-20141023-T00034
Please refer to the end of the specification for access instructions.

Lengthy table referenced here
US20140315844A1-20141023-T00035
Please refer to the end of the specification for access instructions.

Lengthy table referenced here
US20140315844A1-20141023-T00036
Please refer to the end of the specification for access instructions.

Lengthy table referenced here
US20140315844A1-20141023-T00037
Please refer to the end of the specification for access instructions.

Lengthy table referenced here
US20140315844A1-20141023-T00038
Please refer to the end of the specification for access instructions.

Lengthy table referenced here
US20140315844A1-20141023-T00039
Please refer to the end of the specification for access instructions.

Lengthy table referenced here
US20140315844A1-20141023-T00040
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20140315844A1). An electronic copy of the table will also be available from the USPTO upon request and payment of the fee set forth in 37 CFR 1.19(b)(3).

1. A method of determining response to chemotherapy or risk of tumor metastasis for a postmenopausal woman having breast cancer, the method comprising detecting the expression level of the twelve genes CENPA, PKMYT1, MELK, BUB1, RACGAP1, TK1, UBE2S, DC13, RFC4, PRR11, DIAPH3, and ORC6L in a sample of nucleic acid from estrogen receptor (ER)-positive tumor cells from said woman, and identifying said woman as having an increased likelihood of responding to chemotherapy and/or a decreased risk for tumor metastasis if expression levels of said genes are not increased, or identifying said woman as having a decreased likelihood of responding to chemotherapy and/or an increased risk for tumor metastasis if expression levels of said genes are increased.

2. The method of claim 1, further comprising adding together the expression level of each of said genes to obtain a score which indicates said likelihood of responding to chemotherapy and/or said risk of tumor metastasis in said woman.

3. The method of claim 1, wherein the method comprises calculating said expression level as a Δ(ΔCt) value as follows for each of said genes:
Δ(ΔCt)=(Ct_{GOI}−Ct_{EC})_{test RNA}−(Ct_{GOI}−Ct_{EC})_{refRNA}
wherein Ct is the threshold cycle for target amplification, GOI=gene of interest, test RNA=patient sample RNA, ref RNA=reference RNA, and EC=endogenous control.

4. The method of claim 3, further comprising adding together the Δ(ΔCt) values for each of said genes to obtain a score which represents the sum of the Δ(ΔCt) values for all of said genes.

5. The method of claim 4, wherein the Δ(ΔCt) values for each of said genes is equally weighted when added together.

6. The method of claim 4, wherein the Δ(ΔCt) values for each of said genes is weighted by a particular constant value when added together.

7. The method of claim 4, further comprising identifying said woman as having said increased likelihood of responding to chemotherapy and/or said decreased risk of tumor metastasis if said score is low, or identifying said woman as having said decreased likelihood of responding to chemotherapy and/or said increased risk of tumor metastasis if said score is high.

8. The method of claim 7, wherein the method comprises comparing said score to at least one predefined cut-point, and identifying said woman as having said increased likelihood of responding to chemotherapy and/or said decreased risk of tumor metastasis if their score is lower than the predefined cut-point, or identifying said woman as having said decreased likelihood of responding to chemotherapy and/or said increased risk of tumor metastasis if their score is higher than the predefined cut-point.

9. The method of claim 1, wherein said woman is at least 50 years of age or older.

10. The method of claim 1, wherein said breast tumor cells are HER2-negative.

11. The method of claim 1, wherein said woman has no detectable tumor cells in their lymph nodes (node-negative).

12. The method of claim 1, wherein the method comprises reverse transcribing and amplifying mRNA of each of said genes, optionally by using reverse-transcription polymerase chain reaction (RT-PCR).

13. The method of claim 1, wherein the method comprises normalizing the expression levels of said genes against the expression level of at least one control gene, or an average of two or more control genes, optionally wherein the control gene is selected from the group consisting of NUP214, PPIG, SLUT, and any combination thereof.

14. The method of claim 1, wherein said chemotherapy comprises at least one of an anthracycline, a taxane, an inhibitor of nucleotide synthesis, or an alkylating agent.

15. The method of claim 14, wherein said chemotherapy comprises at least one of:
an anthracycline which is at least one of doxorubicin or epirubicin;
a taxane which is at least one of paclitaxel or docetaxel;
an inhibitor of nucleotide synthesis which is at least one of methotrexate or 5-fluorouracil (5-FU); or
an alkylating agent which is cyclophosphamide.

16. The method of claim 1, wherein said chemotherapy is selected from the group consisting of CMF [cyclophosphamide, methotrexate, and fluorouracil (5-FU)], CAF [cyclophosphamide, Adriamycin® (doxorubicin), and fluorouracil (5-FU)], AC [Adriamycin® (doxorubicin) and cyclophosphamide] or AC-Taxol® (AC followed by paclitaxel), TAC [Taxotere® (docetaxel), Adriamycin® (doxorubicin), and cyclophosphamide)], EC (epirubicin and cyclophosphamide), FEC (5-fluorouracil, epirubicin and cyclophosphamide), FECD (FEC followed by docetaxel), TC [Taxotere® (docetaxel) and cyclophosphamide], and MF (methotrexate and fluorouracil (5-FU)).

17. The method of claim 1, wherein said woman has previously been treated with tamoxifen, or wherein said woman if further treated with tamoxifen.

18. The method of claim 1, further comprising administering chemotherapy to said woman who has been identified as having an increased likelihood of responding to chemotherapy.

19. A method of determining response to chemotherapy or risk of tumor metastasis for a human having breast cancer, the method comprising detecting the expression level of genes selected from the group consisting of CENPA, PKMYT1, MELK, MYBL2, BUB1, RACGAP1, TK1, UBE2S, DC13, RFC4, PRR11, DIAPH3, ORC6L and CCNB1 in a sample of nucleic acid from estrogen receptor (ER)-positive tumor cells from said human, and identifying said human as having an increased likelihood of responding to chemotherapy and/or a decreased risk of tumor metastasis if expression levels of said genes are not increased, or identifying said human as having a decreased likelihood of responding to chemotherapy and/or an increased risk of tumor metastasis if expression levels of said genes are increased.

20. The method of claim 19, wherein said method comprises detecting the expression level of a combination of said genes, wherein said combination comprises at least all 14 of said genes or a combination selected from the group consisting of the combinations set forth in Table 40.

21. The method of claim 20, further comprising adding together the expression level of each of the genes of said combination to obtain a score which indicates said likelihood of responding to chemotherapy and/or said risk of tumor metastasis in said human.

22. The method of claim 21, wherein the expression level of each gene of said combination is calculated as a Δ(ΔCt) value, and wherein the method further comprises adding together the Δ(ΔCt) values for each of said genes to obtain a score which represents the sum of the Δ(ΔCt) values for all of the genes of said combination.

23. The method of claim 22, wherein the Δ(ΔCt) values for each of said genes are either equally weighted or weighted by a particular constant value when added together.

24. The method of claim 22, further comprising identifying said human as having said increased likelihood of responding to chemotherapy and/or said decreased risk of tumor metastasis if said score is lower than a predefined cut-point, or identifying said human as having said decreased likelihood of responding to chemotherapy and/or said increased risk of tumor metastasis if said score is higher than said predefined cut-point.

25. A kit for determining response to chemotherapy or risk for tumor metastasis, wherein the kit comprises one or more containers containing an enzyme, a buffer, and reagents for detecting the expression of a combination of genes consisting of all twelve of the genes CENPA, PKMYT1, MELK, BUB1, RACGAP1, TK1, UBE2S, DC13, RFC4, PRR11, DIAPH3, and ORC6L.


SEQUENCE LISTING (34 sequences)

SEQ ID NO: 1 - DNA, 17 bp, Homo Sapiens
catggtggtgccttcaa

SEQ ID NO: 2 - DNA, 23 bp, Homo Sapiens
gctgaatacatgtgagacgacaa

SEQ ID NO: 3 - DNA, 22 bp, Homo Sapiens
gccaaatacctgatggaactaa

SEQ ID NO: 4 - DNA, 20 bp, Homo Sapiens
ctcctgctgcaatttgagaa

SEQ ID NO: 5 - DNA, 16 bp, Homo Sapiens
cagtcggcggagacaa

SEQ ID NO: 6 - DNA, 22 bp, Homo Sapiens
aagaggtgtgtgctcttctgaa

SEQ ID NO: 7 - DNA, 23 bp, Homo Sapiens
aaagtgacctgtgagagattgaa

SEQ ID NO: 8 - DNA, 15 bp, Homo Sapiens
cgccctgcccaacaa

SEQ ID NO: 9 - DNA, 18 bp, Homo Sapiens
ttatcccatcgccttgaa

SEQ ID NO: 10 - DNA, 20 bp, Homo Sapiens
tgctccacaccatgttgtaa

SEQ ID NO: 11 - DNA, 16 bp, Homo Sapiens
agagacgggcccagaa

SEQ ID NO: 12 - DNA, 24 bp, Homo Sapiens
caacagttgatctggattcactaa

SEQ ID NO: 13 - DNA, 15 bp, Homo Sapiens
gcggagccccatcaa

SEQ ID NO: 14 - DNA, 21 bp, Homo Sapiens
catcctcatccacaatgtcaa

SEQ ID NO: 15 - DNA, 20 bp, Homo Sapiens
cacttctgctgcactgcttt

SEQ ID NO: 16 - DNA, 22 bp, Homo Sapiens
ggatgtggctaccattttgttt

SEQ ID NO: 17 - DNA, 18 bp, Homo Sapiens
ctacctgccccctgagtt

SEQ ID NO: 18 - DNA, 21 bp, Homo Sapiens
agcatcatgacaaggacagaa

SEQ ID NO: 19 - DNA, 19 bp, Homo Sapiens
tgtccaagctgtggtcaaa

SEQ ID NO: 20 - DNA, 19 bp, Homo Sapiens
tctccaggggtgatcagaa

SEQ ID NO: 21 - DNA, 18 bp, Homo Sapiens
gactgcgaaaagctggaa

SEQ ID NO: 22 - DNA, 20 bp, Homo Sapiens
ttgctcctcgcttagttgaa

SEQ ID NO: 23 - DNA, 19 bp, Homo Sapiens
tttggcagcagctagagaa

SEQ ID NO: 24 - DNA, 22 bp, Homo Sapiens
cacgttcatcagatgcatttaa

SEQ ID NO: 25 - DNA, 20 bp, Homo Sapiens
gatggtttccacaggaacaa

SEQ ID NO: 26 - DNA, 18 bp, Homo Sapiens
ggatccaagtcccagcaa

SEQ ID NO: 27 - DNA, 18 bp, Homo Sapiens
cctgctgatccaccctaa

SEQ ID NO: 28 - DNA, 23 bp, Homo Sapiens
gcatactcctcgtagttctccaa

SEQ ID NO: 29 - DNA, 20 bp, Homo Sapiens
cactggatcccaagagtgaa

SEQ ID NO: 30 - DNA, 22 bp, Homo Sapiens
tgatcccactccaagtctagaa

SEQ ID NO: 31 - DNA, 22 bp, Homo Sapiens
tggacaagtaatctctggtcaa

SEQ ID NO: 32 - DNA, 19 bp, Homo Sapiens
gtatccgtacctccgcaaa

SEQ ID NO: 33 - DNA, 18 bp, Homo Sapiens
tgccaatgcaggaaagaa

SEQ ID NO: 34 - DNA, 25 bp, Homo Sapiens
tggtatctcctgtgtacctaacaaa